(19) United States
(12) Patent Application Publication
(10) Pub. No.: US 2021/0207218 A1
(43) Pub. Date: Jul. 8, 2021

(54) COMPOSITIONS AND METHODS FOR ASSESSING ACUTE REJECTION IN RENAL TRANSPLANTATION

(71) Applicant: Immucor GTI Diagnostics, Inc., Norcross, GA (US)

(72) Inventor: Minnie M. SARWAL, Mountain View, CA (US)

(21) Appl. No.: 17/119,167

(22) Filed: Dec. 11, 2020

Related U.S. Application Data
(62) Division of application No. 14/916,627, filed on Mar. 4, 2016.
(60) Provisional application No. 61/987,342, filed on May 1, 2014.
(60) Provisional application No. 61/874,970, filed on Sep. 6, 2013.

(51) Int. Cl. C12Q 1/6883

(52) U.S. Cl. CPC C12Q 1/6883; C12Q 2600/158; C12Q 2600/118

(57) ABSTRACT

Provided herein are methods, compositions, and kits for diagnosing acute rejection of renal transplants using the gene expression profile of sets of classifier genes. Such methods and compositions are independent of external confounders such as recipient age, transplant center, RNA source, assay, cause of end-stage renal disease, co-morbidities, immunosuppression usage, and the like.

### CROSS-REFERENCE TO RELATED APPLICATION

This application claims the priority benefit to U.S. Provisional Patent Application Ser. No. 61/874,970 filed Sep. 6, 2013 and U.S. Provisional Patent Application Ser. No. 61/987,342 filed May 1, 2014, the entire content of each is incorporated herein by reference.

### FIELD OF THE INVENTION

The disclosure relates to methods, compositions, and kits for the assessment of acute rejection of renal transplants using the gene expression profile of sets of classifier genes. The described methods and compositions are independent of external confounders such as recipient age, transplant center, RNA source, assay, cause of end-stage renal disease, co-morbidities, immunosuppression usage, and the like.

### BACKGROUND OF THE INVENTION

Organ transplantation from a donor to a host recipient is a component of certain medical procedures and treatment regimes. Following transplantation, it is necessary to avoid graft rejection by the recipient. In order to maintain viability of the donor organ, immunosuppressive therapy is typically employed. Nevertheless, solid organ transplant rejection can still occur.

Organ transplant rejection is classified as hyperacute, acute, borderline acute, subclinical acute, or chronic. For most organs, including kidneys, organ rejection can be unequivocally diagnosed only by performing a biopsy of that organ. For practical reasons, however, biopsies are not always done when acute rejection is suspected. Furthermore, biopsies can be biased by sampling and interpretation (Furness, P. N. et al. Transplantation 2003, 76, 969-973; Furness, P. N. Transplantation 2001, 71, SS31-36) and they are not predictive. Detecting injury in a timely fashion is crucial to ensuring allograft health and long-term survival.

One of the main clinical issues faced by organ transplant recipients is the lack of a sensitive, specific, and non-invasive assay that can be used to serially monitor the patients' alloimmune threshold and risk of acute graft rejection. The rise of highly redundant and non-specific functional markers (e.g. the rise in serum creatinine as a means to indicate graft dysfunction) may suggest acute rejection. However, it has been increasingly recognized (Lerut, E. et al. Transplantation 2007, 83, 1416-1422; Sigdel, T. K. et al. J Am. Soc. Nephrol. 2012, 23, 750-763; Moreso, F. et al. Am. J. Transplant. 2006, 6, 747-752; Moreso, F. et al. Transplantation 2012, 93, 41-46; Heilman, R. L. et al. Am. J. Transplant. 2010, 10, 563-570) that in renal transplantation, injury persists, undetected by a drift in the serum creatinine (subclinical acute rejection), until an unexpected diagnosis at the time of a surveillance biopsy (Racusen, L. C. et al. Kidney International 1999, 55, 713-723; Solez, K. et al. Am. J. Transplant. 2008, 8, 753-760; Naesens, M. et al. Am. J. Transplant. 2012, 12, 2730-2743).

A serial assay that permits detection of acute graft rejection (AR) with high specificity (to reduce invasive protocol biopsies in patients with low risk of AR) and with high sensitivity (to increase clinical surveillance for patients at high risk of AR), earlier than is currently possible, would result in timely clinical intervention in order to mitigate AR, as well as to reduce the immunosuppression protocols for quiescent and stable patients. Many assays are likely to be dependent upon recipient age, co-morbidities, transplant center, immunosuppression usage, and/or cause of end-stage renal disease, and the like. Described herein is a solution to this problem through the development of an assay that is independent of these variables.

All patents, patent applications, publications, documents, and articles cited herein are incorporated herein by reference in their entireties, unless otherwise stated.

### BRIEF SUMMARY OF THE INVENTION

Disclosed herein are compositions and methods for classifying an individual as being at high risk for acute rejection (AR) and/or for being at low risk or no risk for acute rejection (no-AR) of renal transplants. These compositions and methods can be used in such classification in both pediatric and adult patients, comprising the gene expression level of a set of classifier genes.

Accordingly, in one aspect, the invention provides for methods of use in the diagnosis of acute rejection (AR), for use in the diagnosis of no-AR, or for use in the diagnosis of the risk of developing AR in an individual who has received a renal allograft, the method comprising: a) measuring the level of CEACAM4 and between 6 and 16 other genes selected from CFLAR, DUSP1, IFNGR1, ITGAX, MAPK9, NAMPT, NKTR, PSEN1, RNF130, RYBP, EPOR, GZMK, RARA, RHEB, RXRA, and SLC25A37 in a biological sample from said individual to obtain a gene expression result; and b) using a reference standard comprising a single reference expression vector from AR samples for each gene and a single reference expression vector from no-AR samples for each gene, wherein the said gene expression result will be correlated to the reference standards. In any of the embodiments herein, the individual can be an adult aged 23 years or older. In any of the embodiments herein, the individual can be a child or young adult under the age of 23. In any of the embodiments herein, the between 6 and 16 other genes may comprise CFLAR, DUSP1, IFNGR1, ITGAX, MAPK9, NAMPT, NKTR, PSEN1, RNF130, RYBP, EPOR, GZMK, RARA, RHEB, RXRA, and SLC25A37. In any of the embodiments herein, the measuring step may comprise assaying said sample for a gene expression result on a microarray chip. In any of the embodiments herein, the measuring step may comprise assaying said sample for a gene expression result using qPCR. In any of the embodiments herein, the measuring step may comprise assaying said sample for a gene expression result on a bead. In any of the embodiments herein, the measuring step may comprise assaying said sample for a gene expression result on a nanoparticle. In any of the embodiments herein, the measuring step may comprise assaying said sample for a gene expression result on a solid surface which can be porous or non-porous, and can range in size. In any of the embodiments herein, the biological sample can be a whole blood sample. In any of the embodiments herein, the biological sample can be a blood sample. In any of the embodiments herein, the blood sample can be peripheral blood leukocytes. In any of the embodiments herein, the blood sample can be peripheral blood mononuclear cells. In any of the embodiments herein, the comparison of the said gene expression result and the said reference standard may comprise prediction of AR with greater than 70% sensitivity. In any of the embodiments herein, the comparison of the said gene expression result and the said reference standard may comprise prediction of AR with greater than 70% specificity. In any of the embodiments herein, the comparison of the said gene expression result and the said reference standard may comprise prediction of AR with greater than 70% positive predictive value (ppv). In any of the embodiments herein, the comparison of the said gene expression result and the said reference standard may comprise prediction of AR with greater than 70% negative predictive value (npv).

In another aspect, the invention provides for methods of use in the identification of an individual for treatment of acute rejection (AR) of in a renal transplant, the method comprising: a) measuring the level of CEACAM4 and between 6 and 16 other genes selected from CFLAR, DUSP1, IFNGR1, ITGAX, MAPK9, NAMPT, NKTR, PSEN1, RNF130, RYBP, EPOR, GZMK, RARA, RHEB, RXRA, and SLC25A37 in a biological sample from said individual to obtain a gene expression result; and b) using a reference standard comprising a single reference expression vector from AR samples for each gene and a single reference expression vector from no-AR samples for each gene, wherein the said gene expression result will be correlated to the reference standard for the identification. In any of the embodiments herein, the individual can be an adult aged 23 years or older. In any of the embodiments herein, the individual can be a child or young adult under the age of 23. In any of the embodiments herein, the between 6 and 16 other genes may comprise CFLAR, DUSP1, IFNGR1, ITGAX, MAPK9, NAMPT, NKTR, PSEN1, RNF130, RYBP, EPOR, GZMK, RARA, RHEB, RXRA, and SLC25A37. In any of the embodiments herein, the measuring step may comprise assaying said sample for a gene expression result on a microarray chip. In any of the embodiments herein, the measuring step may comprise assaying said sample for a gene expression result on a bead. In any of the embodiments herein, the measuring step may comprise assaying said sample for a gene expression result on a nanoparticle. In any of the embodiments herein, the measuring step may comprise assaying said sample for a gene expression result on a solid surface which can be porous or non-porous, and can range in size. In any of the embodiments herein, the biological sample can be a blood sample. In any of the embodiments herein, the blood sample can be peripheral blood leukocytes. In any of the embodiments herein, the blood sample can be peripheral blood mononuclear cells. In any of the embodiments herein, the biological sample can be a whole blood sample. In any of the embodiments herein, the comparison of the said gene expression result and the said reference standard may comprise prediction of AR with greater than 70% sensitivity. In any of the embodiments herein, the comparison of the said gene expression result and the said reference standard may comprise prediction of AR with greater than 70% specificity. In any of the embodiments herein, the comparing step may comprise prediction of AR with greater than 70% positive predictive value (ppv). In any of the embodiments herein, the comparison of the said gene expression result and the said reference standard may comprise prediction of AR with greater than 70% negative predictive value (npv).

In another aspect, the invention provides for systems for use in diagnosing acute rejection (AR) in an individual who has received a renal allograft, the system comprising: a) a gene expression evaluation element for measuring the level of CEACAM4 and between 6 and 16 other genes selected from CF CFLAR, DUSP1, IFNGR1, ITGAX, MAPK9, NAMPT, NKTR, PSEN1, RNF130, RYBP, EPOR, GZMK, RARA, RHEB, RXRA, and SLC25A37 in a biological sample from said individual to obtain a gene expression result; and b) a reference standard element comprising a single reference expression vector from AR samples for each gene and a single reference expression vector from no-AR samples for each gene, for correlating the said gene expression result to the reference standards for the diagnosis. In any of the embodiments herein, the gene expression evaluation element may comprise a microarray chip. In any of the embodiments herein, the gene expression evaluation element may comprise a bead. In any of the embodiments herein, the gene expression evaluation element may comprise a nanoparticle. In any of the embodiments herein, the measuring step may comprise assaying said sample for a gene expression result on a solid surface which can be porous or non-porous, and can range in size. In any of the embodiments herein, the reference standard element can be computer-generated. In any of the embodiments herein, the said gene expression result to the said reference standard may be performed by a computer or an individual. In any of the embodiments herein, the individual can be an adult aged 23 years or older. In any of the embodiments herein, the individual can be a child or young adult under the age of 23. In any of the embodiments herein, the between 6 and 16 other genes may comprise CFLAR, DUSP1, IFNGR1, ITGAX, MAPK9, NAMPT, NKTR, PSEN1, RNF130, RYBP, EPOR, GZMK, RARA, RHEB, RXRA, and SLC25A37. In any of the embodiments herein, the biological sample can be a blood sample. In any of the embodiments herein, the blood sample can be peripheral blood leukocytes. In any of the embodiments herein, the blood sample can be peripheral blood mononuclear cells. In any of the embodiments herein, the biological sample can be a whole blood sample. In any of the embodiments herein, comparison of the said gene expression result to the said reference standard may predict AR with greater than 70% sensitivity. In any of the embodiments herein, comparison of the said gene expression result to the said reference standard may predict AR with greater than 70% specificity. In any of the embodiments herein, comparison of the said gene expression result to the said reference standard may predict AR with greater than 70% positive predictive value (ppv). In any of the embodiments herein, comparison of the said gene expression result to the said reference standard may predict AR with greater than 70% negative predictive value (npv).

In another aspect, the invention provides for kits for use in diagnosing acute rejection (AR) in an individual who has received a renal allograft, the kit comprising: a) a gene expression evaluation element for measuring the level of CEACAM4 and between 6 and 16 other genes selected from CFLAR, DUSP1, IFNGR1, ITGAX, MAPK9, NAMPT, NKTR, PSEN1, RNF130, RYBP, EPOR, GZMK, RARA, RHEB, RXRA, and SLC25A37 in a biological sample from said individual to obtain a gene expression result; b) a reference standard element comprising a single reference expression vector from AR samples for each gene and a single reference expression vector from no-AR samples for each transplant center; and c) a set of instructions for diagnosing AR, comprising a correlation of the said gene expression result to the reference standards. In any of the embodiments herein, the individual can be an adult aged 23 years or older. In any of the embodiments herein, the individual can be a child or young adult under the age of 23. In any of the embodiments herein, the between 6 and 16 other genes may comprise CFLAR, DUSP1, IFNGR1, ITGAX, MAPK9, NAMPT, NKTR, PSEN1, RNF130, RYBP, EPOR, GZMK, RARA, RHEB, RXRA, and SLC25A37. In any of the embodiments herein, the gene expression evaluation element may comprise assaying said sample for a gene expression result on a microarray chip. In any of the embodiments herein, the gene expression evaluation element may comprise assaying said sample for a gene expression result on a bead. In any of the embodiments herein, the gene expression evaluation element may comprise assaying said sample for a gene expression result on a nanoparticle. In any of the embodiments herein, the measuring step may comprise assaying said sample for a gene expression result on a solid surface which can be porous or non-porous, and can range in size. In any of the embodiments herein, the biological sample can be a blood sample. In any of the embodiments herein, the biological sample can be a whole blood sample. In any of the embodiments herein, the blood sample can be peripheral blood leukocytes. In any of the embodiments herein, the blood sample can be peripheral blood mononuclear cells. In any of the embodiments herein, comparison of the said gene expression result to the said reference standard may predict AR with greater than 70% sensitivity. In any of the embodiments herein, comparison of the said gene expression result to the said reference standard may predict AR with greater than 70% specificity. In any of the embodiments herein, comparison of the said gene expression result to the said reference standard may predict AR with greater than 70% positive predictive value (ppv). In any of the embodiments herein, comparison of the said gene expression result to the said reference standard may predict AR with greater than 70% negative predictive value (npv). In any of the embodiments herein, comparison of the said gene expression result to the said reference standard can be performed by a computer or an individual.

In another aspect, the invention provides for articles of manufacture comprising a reference standard for comparison to a gene expression result obtained by measuring the level of CEACAM4 and between 6 and 16 other genes selected from CFLAR, DUSP1, IFNGR1, ITGAX, MAPK9, NAMPT, NKTR, PSEN1, RNF130, RYBP, EPOR, GZMK, RARA, RHEB, RXRA, and SLC25A37 in a biological sample from an individual who has received a renal allograft, comprising a single reference expression vector from AR samples for each gene at a single renal transplant center and a single reference expression vector from no-AR samples for each gene, wherein the correlation between the said gene expression and the reference standards is for use in the diagnosis of acute rejection (AR), diagnosis of no-AR, or diagnosis of the risk of developing AR in said individual. In any of the embodiments herein, the individual can be an adult aged 23 years or older. In any of the embodiments herein, the individual can be a child or young adult under the age of 23. In any of the embodiments herein, the between 6 and 16 other genes may comprise CFLAR, DUSP1, IFNGR1, ITGAX, MAPK9, NAMPT, NKTR, PSEN1, RNF130, RYBP, EPOR, GZMK, RARA, RHEB, RXRA, and SLC25A37. In any of the embodiments herein, measuring the level of CEACAM4 and between 6 and 16 other genes may comprise assaying said sample for a gene expression result on a microarray chip. In any of the embodiments herein, measuring the level of CEACAM4 and between 6 and 16 other genes may comprise assaying said sample for a gene expression result on a bead. In any of the embodiments herein, measuring the level of CEACAM4 and between 6 and 16 other genes may comprise assaying said sample for a gene expression result on a nanoparticle. In any of the embodiments herein, the measuring step may comprise assaying said sample for a gene expression result on a solid surface which can be porous or non-porous, and can range in size. In any of the embodiments herein, the biological sample is a blood sample. In any of the embodiments herein, the biological sample is a whole blood sample. In any of the embodiments herein, the blood sample can be peripheral blood leukocytes. In any of the embodiments herein, the blood sample can be peripheral blood mononuclear cells. In any of the embodiments herein, the comparison between the said gene expression and the reference standard may comprise prediction of AR with greater than 70% sensitivity. In any of the embodiments herein, the comparison between the said gene expression and the reference standard may comprise prediction of AR with greater than 70% specificity. In any of the embodiments herein, the comparison between the said gene expression and the reference standard may comprise prediction of AR with greater than 70% positive predictive value (ppv). In any of the embodiments herein, the comparison between the said gene expression and the reference standard may comprise prediction of AR with greater than 70% negative predictive value (npv).

In another aspect, the invention provides a method of treatment for renal transplant patients, comprising ordering a test comprising: a) measuring the level of CEACAM4 and between 6 and 16 other genes selected from CFLAR, DUSP1, IFNGR1, ITGAX, MAPK9, NAMPT, NKTR, PSEN1, RNF130, RYBP, EPOR, GZMK, RARA, RHEB, RXRA, and SLC25A37 in a biological sample from said individual to obtain a gene expression result; b) using a reference standard comprising a single reference expression vector from AR samples for each gene and a single reference expression vector from no-AR samples for each gene, wherein the said gene expression result will be compared to the reference standard thereby identifying a subject as having an AR of a renal transplant or not having an AR of a renal transplant; and c) increasing the administration of a therapeutically effective amount of one or more of a therapeutic agent in a subject with an AR of a renal transplant, maintaining the administration of a therapeutically effective amount of one or more of a therapeutic agent in a subject without an AR of a renal transplant, or decreasing the administration of a therapeutically effective amount of one or more of a therapeutic agent in a subject without an AR of a renal transplant. In any of the embodiments herein, the individual can be an adult aged 23 years or older. In any of the embodiments herein, the individual can be a child or young adult under the age of 23. In any of the embodiments herein, the between 6 and 16 other genes may comprise CFLAR, DUSP1, IFNGR1, ITGAX, MAPK9, NAMPT, NKTR, PSEN1, RNF130, RYBP, EPOR, GZMK, RARA, RHEB, RXRA, and SLC25A37. In any of the embodiments herein, the measuring step may comprise assaying said sample for a gene expression result on a microarray chip. In any of the embodiments herein, the measuring step may comprise assaying said sample for a gene expression result on a bead. In any of the embodiments herein, the measuring step may comprise assaying said sample for a gene expression result on a nanoparticle. In any of the embodiments herein, the measuring step may comprise assaying said sample for a gene expression result on a solid surface which can be porous or non-porous, and can range in size. In any of the embodiments herein, the biological sample can be a blood sample. In any of the embodiments herein, the blood sample can be peripheral blood leukocytes. In any of the embodiments herein, the blood sample can be peripheral blood mononuclear cells. In any of the embodiments herein, the biological sample can be a whole blood sample. In any of the embodiments herein, the comparison of the said gene expression result and the said reference standard may comprise prediction of AR with greater than 70% sensitivity. In any of the embodiments herein, the comparison of the said gene expression result and the said reference standard may comprise prediction of AR with greater than 70% specificity. In any of the embodiments herein, the comparing step may comprise prediction of AR with greater than 70% positive predictive value (ppv). In any of the embodiments herein, the comparison of the said gene expression result and the said reference standard may comprise prediction of AR with greater than 70% negative predictive value (npv).

In another aspect, the invention provides for methods of use in the diagnosis of no acute rejection (no-AR) in an individual who has received a renal allograft, the method comprising: a) measuring the level of CEACAM4 and between 6 and 16 other genes selected from CFLAR, DUSP1, IFNGR1, ITGAX, MAPK9, NAMPT, NKTR, PSEN1, RNF130, RYBP, EPOR, GZMK, RARA, RHEB, RXRA, and SLC25A37 in a biological sample from said individual to obtain a gene expression result; and b) using a reference standard comprising a single reference expression vector from AR samples for each gene and a single reference expression vector from no-AR samples for each gene, wherein the said gene expression result will be correlated to the reference standards. In any of the embodiments herein, the individual can be an adult aged 23 years or older. In any of the embodiments herein, the individual can be a child or young adult under the age of 23. In any of the embodiments herein, the between 6 and 16 other genes may comprise CFLAR, DUSP1, IFNGR1, ITGAX, MAPK9, NAMPT, NKTR, PSEN1, RNF130, RYBP, EPOR, GZMK, RARA, RHEB, RXRA, and SLC25A37. In any of the embodiments herein, the measuring step may comprise assaying said sample for a gene expression result on a microarray chip. In any of the embodiments herein, the measuring step may comprise assaying said sample for a gene expression result on a bead. In any of the embodiments herein, the measuring step may comprise assaying said sample for a gene expression result on a nanoparticle. In any of the embodiments herein, the measuring step may comprise assaying said sample for a gene expression result on a solid surface which can be porous or non-porous, and can range in size. In any of the embodiments herein, the biological sample can be a whole blood sample. In any of the embodiments herein, the biological sample can be a blood sample. In any of the embodiments herein, the blood sample can be peripheral blood leukocytes. In any of the embodiments herein, the blood sample can be peripheral blood mononuclear cells. In any of the embodiments herein, the comparison of the said gene expression result and the said reference standard may comprise prediction of no-AR with greater than 70% sensitivity. In any of the embodiments herein, the comparison of the said gene expression result and the said reference standard may comprise prediction of no-AR with greater than 70% specificity. In any of the embodiments herein, the comparison of the said gene expression result and the said reference standard may comprise prediction of no-AR with greater than 70% positive predictive value (ppv). In any of the embodiments herein, the comparison of the said gene expression result and the said reference standard may comprise prediction of no-AR with greater than 70% negative predictive value (npv).

In another aspect, the invention provides for methods of use in the identification of an individual for treatment of no acute rejection (no-AR) in a renal transplant, the method comprising: a) measuring the level of CEACAM4 and between 6 and 16 other genes selected from CFLAR, DUSP1, IFNGR1, ITGAX, MAPK9, NAMPT, NKTR, PSEN1, RNF130, RYBP, EPOR, GZMK, RARA, RHEB, RXRA, and SLC25A37 in a biological sample from said individual to obtain a gene expression result; and b) using a reference standard comprising a single reference expression vector from AR samples for each gene at a single renal transplant center and a single reference expression vector from no-AR samples for each gene at a single renal transplant center, wherein the said gene expression result will be correlated to the reference standards for the identification. In any of the embodiments herein, the individual can be an adult aged 23 years or older. In any of the embodiments herein, the individual can be a child or young adult under the age of 23. In any of the embodiments herein, the between 6 and 16 other genes may comprise CFLAR, DUSP1, ITGAX, NAMPT, NKTR, PSEN1, EPOR, GZMK, RARA, RHEB, and SLC25A37. In any of the embodiments herein, the measuring step may comprise assaying said sample for a gene expression result on a microarray chip. In any of the embodiments herein, the measuring step may comprise assaying said sample for a gene expression result on a bead. In any of the embodiments herein, the measuring step may comprise assaying said sample for a gene expression result on a nanoparticle. In any of the embodiments herein, the measuring step may comprise assaying said sample for a gene expression result on a solid surface which can be porous or non-porous, and can range in size. In any of the embodiments herein, the biological sample can be a whole blood sample. In any of the embodiments herein, the biological sample can be a blood sample. In any of the embodiments herein, the blood sample can be peripheral blood leukocytes. In any of the embodiments herein, the blood sample can be peripheral blood mononuclear cells. In any of the embodiments herein, the comparison of the said gene expression result and the said reference standard may comprise prediction of no-AR with greater than 70% sensitivity. In any of the embodiments herein, the comparison of the said gene expression result and the said reference standard may comprise prediction of no-AR with greater than 70% specificity. In any of the embodiments herein, the comparing step may comprise prediction of no-AR with greater than 70% positive predictive value (ppv). In any of the embodiments herein, the comparison of the said gene expression result and the said reference standard may comprise prediction of no-AR with greater than 70% negative predictive value (npv).

In another aspect, the invention provides for systems for use in diagnosing no acute rejection (no-AR) in an individual who has received a renal allograft, the system comprising: a) a gene expression evaluation element for measuring the level of CEACAM4 and between 6 and 16 other genes selected from CFLAR, DUSP1, IFNGR1, ITGAX, MAPK9, NAMPT, NKTR, PSEN1, RNF130, RYBP, EPOR, GZMK, RARA, RHEB, RXRA, and SLC25A37 in a biological sample from said individual to obtain a gene expression result; and b) a reference standard element comprising a single reference expression vector from AR samples for each gene at a single renal transplant center and a single reference expression vector from no-AR samples for each gene at a single renal transplant center, for correlating the said gene expression result to the reference standards for the diagnosis. In any of the embodiments herein, the gene expression evaluation element may comprise a microarray chip. In any of the embodiments herein, the gene expression evaluation element may comprise a bead. In any of the embodiments herein, the gene expression evaluation element may comprise a nanoparticle. In any of the embodiments herein, the measuring step may comprise assaying said sample for a gene expression result on a solid surface which can be porous or non-porous, and can range in size. In any of the embodiments herein, the reference standard element can be computer-generated. In any of the embodiments herein, the said gene expression result to the said reference standard may be performed by a computer or an individual. In any of the embodiments herein, the individual can be an adult aged 23 years or older. In any of the embodiments herein, the individual can be a child or young adult under the age of 23. In any of the embodiments herein, the between 6 and 16 other genes may comprise CFLAR, DUSP1, IFNGR1, ITGAX, MAPK9, NAMPT, NKTR, PSEN1, RNF130, RYBP, EPOR, GZMK, RARA, RHEB, RXRA, and SLC25A37. In any of the embodiments herein, the biological sample can be a whole blood sample. In any of the embodiments herein, the biological sample can be a blood sample. In any of the embodiments herein, the blood sample can be peripheral blood leukocytes. In any of the embodiments herein, the blood sample can be peripheral blood mononuclear cells. In any of the embodiments herein, comparison of the said gene expression result to the said reference standard may predict no-AR with greater than 70% sensitivity. In any of the embodiments herein, comparison of the said gene expression result to the said reference standard may predict no-AR with greater than 70% specificity. In any of the embodiments herein, comparison of the said gene expression result to the said reference standard may predict no-AR with greater than 70% positive predictive value (ppv). In any of the embodiments herein, comparison of the said gene expression result to the said reference standard may predict no-AR with greater than 70% negative predictive value (npv).

In another aspect, the invention provides for kits for use in diagnosing no acute rejection (no-AR) in an individual who has received a renal allograft, the kit comprising: a) a gene expression evaluation element for measuring the level of CEACAM4 and between 6 and 16 other genes selected from CFLAR, DUSP1, IFNGR1, ITGAX, MAPK9, NAMPT, NKTR, PSEN1, RNF130, RYBP, EPOR, GZMK, RARA, RHEB, RXRA, and SLC25A37 in a biological sample from said individual to obtain a gene expression result; b) a reference standard element comprising a single reference expression vector from AR samples for each gene at a single renal transplant center and a single reference expression vector from no-AR samples for each gene at a single renal transplant center; and c) a set of instructions for diagnosing AR, comprising a correlation of the said gene expression result to the reference standards. In any of the embodiments herein, the individual can be an adult aged 23 years or older. In any of the embodiments herein, the individual can be a child or young adult under the age of 23. In any of the embodiments herein, the between 6 and 16 other genes may comprise CFLAR, DUSP1, IFNGR1, ITGAX, MAPK9, NAMPT, NKTR, PSEN1, RNF130, RYBP, EPOR, GZMK, RARA, RHEB, RXRA, and SLC25A37. In any of the embodiments herein, the gene expression evaluation element may comprise assaying said sample for a gene expression result on a microarray chip. In any of the embodiments herein, the gene expression evaluation element may comprise assaying said sample for a gene expression result on a bead. In any of the embodiments herein, the gene expression evaluation element may comprise assaying said sample for a gene expression result on a nanoparticle. In any of the embodiments herein, the measuring step may comprise assaying said sample for a gene expression result on a solid surface which can be porous or non-porous, and can range in size. In any of the embodiments herein, the biological sample can be a whole blood sample. In any of the embodiments herein, the biological sample can be a blood sample. In any of the embodiments herein, the blood sample can be peripheral blood leukocytes. In any of the embodiments herein, the blood sample can be peripheral blood mononuclear cells. In any of the embodiments herein, comparison of the said gene expression result to the said reference standard may predict no-AR with greater than 70% sensitivity. In any of the embodiments herein, comparison of the said gene expression result to the said reference standard may predict no-AR with greater than 70% specificity. In any of the embodiments herein, comparison of the said gene expression result to the said reference standard may predict no-AR with greater than 70% positive predictive value (ppv). In any of the embodiments herein, comparison of the said gene expression result to the said reference standard may predict no-AR with greater than 70% negative predictive value (npv). In any of the embodiments herein, comparison of the said gene expression result to the said reference standard can be performed by a computer or an individual.

In another aspect, the invention provides for articles of manufacture comprising a reference standard for comparison to a gene expression result obtained by measuring the level of CEACAM4 and between 6 and 16 other genes selected from CFLAR, DUSP1, IFNGR1, ITGAX, MAPK9, NAMPT, NKTR, PSEN1, RNF130, RYBP, EPOR, GZMK, RARA, RHEB, RXRA, and SLC25A37 in a biological sample from an individual who has received a renal allograft, comprising a single reference expression vector from AR samples for each gene at a single renal transplant center and a single reference expression vector from no-AR samples for each gene at a single renal transplant center, wherein the correlation between the said gene expression and the reference standards is for use in the diagnosis of no acute rejection (no-AR) in said individual. In any of the embodiments herein, the individual can be an adult aged 23 years or older. In any of the embodiments herein, the individual can be a child or young adult under the age of 23. In any of the embodiments herein, the between 6 and 16 other genes may comprise CFLAR, DUSP1, IFNGR1, ITGAX, MAPK9, NAMPT, NKTR, PSEN1, RNF130, RYBP, EPOR, GZMK, RARA, RHEB, RXRA, and SLC25A37. In any of the embodiments herein, measuring the level of CEACAM4 and between 6 and 16 other genes may comprise assaying said sample for a gene expression result on a microarray chip. In any of the embodiments herein, measuring the level of CEACAM4 and between 6 and 16 other genes may comprise assaying said sample for a gene expression result on a bead. In any of the embodiments herein, measuring the level of CEACAM4 and between 6 and 16 other genes may comprise assaying said sample for a gene expression result on a nanoparticle. In any of the embodiments herein, the measuring step may comprise assaying said sample for a gene expression result on a solid surface which can be porous or non-porous, and can range in size. In any of the embodiments herein, the biological sample is a whole blood sample. In any of the embodiments herein, the biological sample is a blood sample. In any of the embodiments herein, the blood sample can be peripheral blood leukocytes. In any of the embodiments herein, the blood sample can be peripheral blood mononuclear cells. In any of the embodiments herein, the comparison between the said gene expression and the reference standard may comprise prediction of no-AR with greater than 70% sensitivity. In any of the embodiments herein, the comparison between the said gene expression and the reference standard may comprise prediction of no-AR with greater than 70% specificity. In any of the embodiments herein, the comparison between the said gene expression and the reference standard may comprise prediction of no-AR with greater than 70% positive predictive value (ppv). In any of the embodiments herein, the comparison between the said gene expression and the reference standard may comprise prediction of no-AR with greater than 70% negative predictive value (npv).

### BRIEF DESCRIPTION OF THE DRAWINGS

FIG. 1 describes the Assessment of Acute Rejection in Renal Transplantation (AART) Study Design in 438 unique adult/pediatric renal transplant patients from 8 transplant centers worldwide.

FIGS. 2A-B are graphs showing prediction of acute rejection (AR) in 192 patients from 4 centers using 15 genes via penalized logistic regression.

FIG. 3A is a graph showing that 15 genes detect cellular and humoral rejection via penalized logistic regression. FIG. 3B illustrates that detection of AR and no-AR using 15 genes via penalized logistic regression is not confounded by time post-transplantation.

FIGS. 4A-B show the predicted probabilities of AR for 156 pediatric and adult samples collected 2 years to 0 months prior to a biopsy-proven AR episode or 0-16 months after a biopsy-proven AR episode. FIG. 4A shows that expression of 15 genes in the adult sample population indicates AR up to 3 months before and until 1 month after the biopsy for AR via penalized logistic regression. FIG. 4B shows that expression of 5 of the 10 genes predict AR in the adult sample population up to 3 months prior and after the AR biopsy via logistic regression.

FIGS. 5A-B depict the workflow of the modified lineage profiler (kSAS). FIG. 5A illustrates that samples can be classified based on overall similarity to AR and STA references without the need for batch effect correction. FIG. 5B shows how kSAS (modified Lineage Profiler) fits in the workflow from qPCR data to an AR Relative Risk Model.

FIGS. 6A-B describes the Classification of AR and No-AR in 143 adult samples using 17 genes via partial least square Discriminant analysis (plsDA). The 17 genes were used to predict AR in 143 adult blood samples (Cohort 1) from four sites by plsDA. 6A shows the mean [%] predicted probabilities for AR vs. No-AR in each collection site were significantly higher in AR in each site (p<0.0001), and did not reach the threshold for AR prediction in the No-AR samples (predicted probability AR=50%). 6B shows the receiver operating characteristic (ROC) AUC for AR in the training set was 0.94 (95% CI 0.91-0.98).

FIGS. 7A-C shows the Classification of AR and No-AR in 124 adult and pediatric samples using the 17 genes. Independent validation in 124 adult and pediatric AR and No-AR blood samples (Cohort 2) using the fixed plsDA 17-gene model on Fluidigm. 22/23 AR correctly classified as AR and 100/101 No-AR correctly classified as No-AR. 7A: [%] predicted AR probabilities segregated by phenotype (AR vs. No-AR) and patient age (adult; pediatric) are shown for each sample. 7B: Mean predicted AR probability across all samples was significantly higher in AR vs. No-AR (p<0.0001). 7C: ROC analyses for the 17 gene AR model demonstrated high sensitivity and specificity for AR prediction (AUC=0.95 [95% CI 0.88 to 1.0]).

FIG. 8 shows the prediction of AR in 191 adult and pediatric samples using 17 genes. 191 serial blood samples (Cohort 3) were profiled within 6 months before (pre-AR) or after (post-AR) biopsy confirmed AR. Mean incidence of AR and No-AR is shown in each group including 74 AR samples, and 117 pre- and post-AR biopsy samples, and 216 No-AR/stable samples. Within columns, mean predicted probability scores of AR calculated by the assay are shown. The 17 gene kidney AR prediction model predicted AR in 62.9% of samples collected within 3 months pre-AR with very high mean AR scores (96.4%±0.08). AR scores persisted in 51.6% of samples collected <3 months post-AR, again with very high mean predicted AR scores (94.6%±0.14); 83.8% of the No-AR samples were always predicted as No-AR (mean predicted AR probability=8.2%±0.12). Mean AR scores were significantly different between pre-AR samples (0-3 months) vs. No-AR/stable samples (p=3.72E-47).

FIGS. 9A-C shows the development of the kSAS algorithm using 17 genes. kSAS was developed to provide individual sample AR risk scores and AR risk categories. FIG. 9A shows expression values of the 17 gene kidney AR prediction assay model in unknown samples were correlated to corresponding AR and No-AR reference values by Pearson Correlation;

FIG. 9B shows for the 17 gene AR assay development QPCR data from 100 samples were divided into Training (n=32) and independent Validation Sets (n=68); 13 12-gene models from the 17 gene kidney AR prediction assay model generated numerical aggregated AR Risk Scores for each sample and categorized them into three groups High-Risk AR (aggregated AR risk score ≥9), Low-Risk AR (aggregated AR risk-score ≤−9) and into an indeterminate (aggregated AR risk-scores <9, and >−9) category 9C.

FIGS. 10A-C shows the performance of the 17 gene AR prediction assay in 100 samples using kSAS. FIG. 10A shows predicted aggregated AR risk scores were calculated for each samples: the AR prediction assay correctly classified 36/39 AR as High-Risk AR (92.3%; Risk-score ≥9) and 43/46 No-AR as Low-Risk AR (93.5%, Risk-Score ≤−9) across 4 different sample collection sites, and adult/pediatric recipient ages; remaining 11 samples classified indeterminate (Risk-Score <9, >−9). FIG. 10B shows an aggregated AR-Risk scores [%] were significantly higher in AR vs. No-AR (p<0.0001). FIG. 10C shows that the ROC analysis demonstrated high sensitivity and specificity for the AR prediction assay; AUC=0.93 (95% CI 0.86-0.9).

FIGS. 11A-D show the confounder analysis and data normalization in Fluidigm QPCR data. Principal component analysis (PCA) of QPCR data from 143 AR and No-AR adult samples (Cohort 1) for 43 rejection genes revealed sample segregation by sample collection site (FIG. 11A) rather than phenotype (FIG. 11B). Normalization of QPCR data by mixed ANOVA corrected for the dominant effect of sample collection site on gene expression (FIG. 11C) and resulted in segregation of samples into AR and No-AR (FIG. 11D). PCA was performed using relative gene expression values (dCt 18S) for 43 genes. A mixed ANOVA model was built with sample collection site, RNA source and chip as random categorical factors and phenotype as categorical factor. Each sphere represents a sample; symbols reflect sample collection sites (*=UPMC; Δ=UCLA; X=CPMC; #=EMORY); the figure also reflects patient phenotype (AR; No-AR) based on biopsy diagnosis.

FIG. 12 shows the methods for identification of AR and No-AR specific genes in 267 adult and pediatric samples. Discovery of the final 17 kidney AR genes for AR prediction was done in gene expression data from 267 adult and pediatric blood samples (Cohort 1, Cohort 2) from the microfluidic high throughput Fluidigm QPCR performed for a total of 43 genes: 10 pediatric AR genes previously identified by us; 33 candidate genes for novel discovery in adult and pediatric transplant rejection. Confirming the pediatric 10 genes in the adult set of 143 AR and No-AR samples correctly predicted AR with 87.4%. Novel discovery and validation was performed in the combined adult and pediatric data set of 267 AR and No-AR samples (Cohort 1, Cohort 2). Student T-test, ANOVA and penalized logistic regression resulting in the definition of 7 additional genes which together with the 10 rejection set defined the final selection of 17 genes for AR prediction. By partial least square discriminant analysis with equal prior probabilities the 17 genes predicted AR with high sensitivity and specificity in the training set of 143 Samples (Cohort 1; AUC=0.944) as well as in the independent Validation set of 124 samples (Cohort 2) not included in any previous analysis (AUC=0.948). Gene expression data used in the analysis represented dCt values against 18S from the Fluidigm QPCR platform additionally normalized for sample collection site, RNA source, and run using a mixed ANOVA model.

FIGS. 13A-D show the individual classifications of AR and No-AR in each participating Center using 17 genes. ROC analyses were performed for each transplant center included in the AART study to assess the performance of kidney AR prediction assay across different sample collection sites. Calculated AUCs were 0.8765 (95% CI 0.7538 to 0.9993) for AR vs. No-AR collected at Emory University (FIG. 13A; n=42); 0.9825 (95% CI 0.9608 to 1.0) for AR vs. No-AR collected at UPMC (13B; n=81), 0.9360 (95% CI 0.8648 to 1.0) for AR vs. No-AR collected at UCLA (13C, n=44), and 1.0 (95% CI 1.0 to 1.0) for AR vs. No-AR collected at CPMC (FIG. 13D, n=35). The latter is an imbalanced data-set with only 2 AR samples and kidney AR prediction assay performance likely over fitted. Tables next to each ROC curve displays the constellation of samples in each Center evaluated.

FIGS. 14A-B show that 17 genes detect antibody and cellular mediated AR via plsDA and the AR and No-AR classification is independent of time post transplantation. FIG. 14A shows the predicted probabilities of AR by the fixed 17 gene kidney AR prediction assay model is compared in a subset of 19 patients with clear antibody mediated rejection only (AMR, C4D positive biopsy staining, DSA+) to a subset of 51 patients with clean cellular mediated rejection (ACR, C4d- and DSA−); the fixed 17 gene model equally detects humoral and cellular AR (14A, plsDA, p=0.9906; mean ACR=80.84%±4.4; mean AMR=80.75%±6.6). FIG. 14B shows that similarly the 17 fixed gene plsDA model predicted AR independent of time post transplantation with continuous low predicted probabilities for AR in the No-AR patients and continuous high AR predicted probabilities in the AR patient group (FIG. 14B shows mean predicted probability of AR plus SEM). Mean AR predicted probabilities were calculated for sample falling in 1 of 3 time post transplantation categories (0-6 months, 6 months-1 year, >1 year) and compared by Student T-test; p values did not reach significance (p>0.05).

FIGS. 15A-C show the biological basis of the 17 genes. Pathway and Network analyses demonstrated strong biological correlation of genes supporting correlation seen in gene expression across AR and No-AR samples by QPCR. FIG. 15A shows significantly (p<0.05) associated with the 17 genes were regulation of apoptosis, immune phenotype and cell surface proteins; FIG. 15B shows the Ingenuity Pathway Analyses (IPA, Qiagen, Redwood City, Calif.) further demonstrated a common role of 11 of the 17 genes in cancer, cell death and cell survival (p<0.05). FIG. 15C shows that additional network analyses showed that 7 of the 17 genes formed a single network of direct interactions.

FIG. 16 shows 12 genes found to be overexpressed in organ transplant rejections representing a common rejection module across multiple different types of organ transplant rejections.

### DETAILED DESCRIPTION OF THE INVENTION

The inventors have discovered groups of gene expression profiles that can determine whether an individual who has received a renal transplant is undergoing, or will undergo, acute rejection (AR) of the transplanted organ. The gene expression profiles are independent of recipient age, transplant center, RNA source, assay, cause of end-stage renal disease, co-morbidities, immunosuppression usage and the like. The invention described herein provides methods for assessing AR or no-AR in an individual who has received a renal allograft, as well as methods of identifying an individual for treatment of AR in a renal transplant. The invention also describes systems for assessing AR in a renal allograft, including the use of microarray chips as components of these systems. The invention further provides for kits based on these systems to assess AR and the probability of AR in an individual who has received a renal allograft.

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

“Acute rejection” “acute allograft rejection” or “AR” is the rejection by the immune system of a tissue/organ transplant recipient when the transplanted tissue is immunologically foreign. AR can be characterized by infiltration of the transplanted tissue by immune cells of the recipient, which carry out their effector function and destroy the transplanted tissue. AR can also be characterized by development of donor-specific antibodies, a diagnosis referred to as antibody-mediated rejection (AMR). AR can be further classified as hyperacute, acute, borderline acute, or subclinical AR. The onset of hyperacute rejection is generally rapid and generally occurs in humans within minutes to hours after transplant surgery. The onset of AR generally occurs in humans within months, often approximately 6-12 months after transplant surgery. Borderline acute and subclinical AR are the result of low grade inflammatory alloresponses. Generally, AR can be treated, inhibited, or suppressed with immunosuppressive drugs such as rapamycin, cyclosporine A, anti-CD40L monoclonal antibodies, and the like.

“No acute rejection” or “no-AR” or “Stable” or “STA” is used interchangeably herein. No-AR/STA represents a patient at low risk or no risk of AR following transplantation. No-AR can be characterized by the long-term graft survival of transplanted tissue that is immunologically foreign to a tissue transplant recipient.

The term “renal allograft” refers to a kidney transplant from one individual to another individual.

As used herein, “gene” refers to a nucleic acid comprising an open reading frame encoding a polypeptide, including exon and (optionally) intron sequences. The term “intron” refers to a DNA sequence present in a given gene that is not translated into protein and is generally found between exons in a DNA molecule. In addition, a gene may optionally include its natural promoter (i.e., the promoter with which the exon and introns of the gene are operably linked in a non-recombinant cell), and associated regulatory sequences, and may or may not include sequences upstream of the AUG start site, untranslated leader sequences, signal sequences, downstream untranslated sequences, transcriptional start and stop sequences, polyadenylation signals, translational start and stop sequences, ribosome binding sites, and the like.

The term “reference” refers to a known value or set of known values against which an observed value may be compared. In one embodiment, the reference is the value (or level) of gene expression of a gene in a graft survival phenotype. In another embodiment, the reference is the value (or level) of gene expression of a gene in a graft loss phenotype.

As used herein, “reference expression vector” refers to a reference standard. In one embodiment, the reference expression vector is a reference standard created for AR samples for each expressed gene at a given transplant center. In another embodiment, the reference expression vector is a reference standard created for no-AR samples for each expressed gene at a given transplant center. In another embodiment, the reference expression vector is a reference standard created for AR samples for each expressed gene across transplant centers. In another embodiment, the reference expression vector is a reference standard created for no-AR samples for each expressed gene across transplant centers.

An “individual” or “subject” can be a “patient.” A “patient” refers to an “individual” who is under the care of a treating physician. The patient can be male or female. In one embodiment, the patient has received a kidney transplant. In another embodiment, the patient has received a kidney transplant and is underdoing organ rejection. In yet another embodiment, the patient has received a kidney transplant and is undergoing AR.

A “patient sub-population,” and grammatical variations thereof, as used herein, refers to a patient subset characterized as having one or more distinctive measurable and/or identifiable characteristics that distinguishes the patient subset from others in the broader disease category to which it belongs.

The term “sample,” as used herein, refers to a composition that is obtained or derived from an individual that contains genomic information. In one embodiment, the sample is whole blood. In one embodiment, the sample is blood. In another embodiment, the sample is peripheral blood leukocytes. In another embodiment, the sample is peripheral blood mononuclear cells. In another embodiment, the sample is a tissue biopsy. In another embodiment, the sample is a tissue biopsy from a transplanted organ. In another embodiment, the sample is a tissue biopsy from an organ prior to transplantation in a recipient.

As used herein, “microarray” refers to an arrangement of a collection of nucleotide sequences in a centralized location. Arrays can be on a solid substrate, such as a surface composed of glass, plastic, or silicon. The nucleotide sequences can be DNA, RNA, or any permutation thereof. The nucleotide sequences can also be partial sequences from a gene, primers, whole gene sequences, non-coding sequences, coding sequences, published sequences, known sequences, or novel sequences.

“Predicting” and “prediction” as used herein does not mean that the outcome is occurring with 100% certainty. Instead, it is intended to mean that the outcome is more likely occurring than not. Acts taken to “predict” or “make a prediction” can include the determination of the likelihood that an outcome is more likely occurring than not. Assessment of multiple factors described herein can be used to make such a determination or prediction.

By “compare” or “comparing” is meant correlating, in any way, the results of a first analysis with the results of a second and/or third analysis. For example, one may use the results of a first analysis to classify the result as more similar to a second result than to a third result. With respect to the embodiment of AR assessment of biological samples from an individual, one may use the results to determine whether the individual is undergoing an AR response. With respect to the embodiment of no-AR assessment of biological samples from an individual, one may use the results to determine whether the individual is undergoing a no-AR response.

The terms “assessing” and “determining” are used interchangeably to refer to any form of measurement, and include both quantitative and qualitative measurements. For example, “assessing” may be relative or absolute.

The term “diagnosis” is used herein to refer to the identification or classification of a molecular or pathological state, disease, or condition. For example, “diagnosis” may refer to identification of an organ rejection. “Diagnosis” may also refer to the classification of a particular sub-type of organ rejection, such as AR.

As used herein, “treatment” refers to clinical intervention in an attempt to alter the natural course of the individual being treated. Desirable effects of treatment include preventing the occurrence or recurrence of a disease or a condition or symptom thereof, alleviating a condition or symptom of the disease, diminishing any direct or indirect pathological consequences of the disease, decreasing the rate of disease progression, ameliorating or palliating the disease state, and achieving improved prognosis. In certain embodiments, treatment refers to decreasing the rate of disease progression, ameliorating or palliating the disease state, and achieving improved prognosis of AR in an individual. In some embodiments, treatment refers to a clinical intervention that modifies or changes the administration a treatment regimen of one or more of a therapeutic agent in a subject.

Reference to “about” a value or parameter herein includes (and describes) embodiments that are directed to that value or parameter per se. For example, description referring to “about X” includes description of “X”. The term “about” is used to provide flexibility to a numerical range endpoint by providing that a given value may be “a little above” or “a little below” the endpoint without affecting the desired result. Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

It is understood that aspects and embodiments of the invention described herein include “comprising,” “consisting,” and “consisting essentially of” aspects and embodiments. For all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as ‘consisting essentially of’ a component, the composition may additionally contain any amount of pharmaceutically acceptable carriers, vehicles, or diluents and other such components which do not substantially affect the condition being treated.

As used in the specification and the appended claims, the singular forms “a,” “an,” and “the” include plural referents unless the context clearly indicates otherwise.

### General Techniques

Unless defined otherwise, technical and scientific terms used herein have the same meaning as commonly understood by one of ordinary skill in the art to which this invention belongs.

The practice of the present invention will employ, unless otherwise indicated, conventional techniques of protein biology, protein chemistry, molecular biology (including recombinant techniques), microbiology, cell biology, biochemistry, and immunology, which are within the skill of the art. Such techniques are explained fully in the literature, such as “Molecular Cloning: A Laboratory Manual”, second edition (Sambrook et al., 1989); “Current Protocols in Molecular Biology” (Ausubel et al., eds., 1987, periodic updates); “PCR: The Polymerase Chain Reaction”, (Mullis et al., eds., 1994); and Singleton et al., Dictionary of Microbiology and Molecular Biology, 2nd ed., J. Wiley & Sons (New York, N.Y. 1994).

### Renal Allograft Recipients

The renal allograft recipient may be of any age. In some embodiments, the individual is a child. In one embodiment, the child is an infant. In another embodiment, the child is a toddler. In other embodiments, the individual is a young adult under the age of 23. In some embodiments, the individual is approximately 1, 2, 3, 4, 5, 6, 7, 8, 9, 10, 11, 12, 13, 14, 15, 16, 17, 18, 19, 20, 21, or 22 years of age. In further embodiments, the individual is an adult over the age of 23. In some embodiments, the individual is approximately 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 67, 68, 69, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years of age. In one embodiment, the renal allograft recipient is female. In another embodiment, the renal allograft recipient is male.

The renal transplant operation/surgery may take place at a specially-designated treatment facility or transplant center. The transplant center may be located anywhere in the world. In one embodiment, the transplant center is in the United States of America. In some embodiments, the transplant center is Emory University (Atlanta, Ga.), the University of California Los Angeles (Los Angeles, Calif.), the University of Pittsburgh (Pittsburgh, Pa.), the California Pacific Medical Center (San Francisco, Calif.), or the University of California San Francisco (San Francisco, Calif.). In other embodiments, the transplant center is in Europe. In one embodiment, the transplant center is University Hospital (Barcelona, Spain). In a further embodiment, the transplant center is in Mexico. In one embodiment, the transplant center is the Laboratorio de Investigacion en Nefrologia, Hospital Infantil de Mexico (Mexico City, Mexico).

Collection of Biological Samples from Renal Allograft Recipients

A biological sample is collected from an individual who has received a renal allograft transplant. In some embodiments, the renal allograft recipient has no outward symptoms of AR. In other embodiments, the renal allograft recipient shows symptoms of AR. Any type of biological sample may be collected, including but not limited to whole blood, blood, serum, plasma, urine, mucus, saliva, cerebrospinal fluid, tissues, biopsies and combinations thereof. In one embodiment, the biological sample is whole blood. In one embodiment, the biological sample is blood. In some embodiments, the blood sample is peripheral blood. In another embodiment, the biological sample is peripheral blood mononuclear cells. In some embodiments, the biological sample is peripheral blood lymphocytes. In some embodiments, the biological sample is a tissue biopsy.

Collection of a biological sample from a renal allograft recipient can occur at any time following the organ transplant. In some embodiments, biological samples can be collected in PAXgene™ tubes (available from Qiagen). In other embodiments, biological samples can be collected in collection tubes that contain RNase inhibitors to prevent RNA degradation. In some embodiments, the biological sample is collected during routine protocol surveillance examination. In other embodiments, the biological sample is collected when a treating clinician has reason to suspect that the individual is undergoing an AR response.

The biological sample that is collected from a renal allograft recipient may be paired with a contemporaneous renal allograft biopsy from the same patient when creating a reference for AR or no-AR samples. Typically, the renal allograft biopsy is collected from the recipient within 48 hours of the biological sample collection. In some embodiments, the biopsy is collected at the time of engraftment. In other embodiments, the biopsy is collected up to 24 months post-transplantation. In one embodiment, the biopsy may be collected at about 3 months post-transplantation; at about 6 months post-transplantation; at about 12 months post-transplantation; at about 18 months post-transplantation; or at about 24 months post-transplantation. These time points should not be seen as limiting, as a biopsy and/or biological sample may be collected at any point following transplantation. Rather, these time points are provided to demonstrate periods following transplantation when routine surveillance is most likely to occur in a majority of renal allograft recipients. In addition, these time points demonstrate periods following transplantation when an AR response is most likely to occur.

Each renal allograft biopsy that is collected may be scored according to the Banff classification system (Solez, K. et al. Am. J. Transplant., 2008, 8, 753-760; Mengel, M. et al. Am. J. Transplant. 2012, 12, 563-570). This system classifies the observed pathology of a renal organ biopsy sample as normal histology, hyperacute rejection, borderline changes, acute rejection, chronic allograft nephropathy, and other changes. The Banff classification sets standards in renal transplant pathology and is widely used in international clinical trials of new anti-rejection agents. As described herein, “acute rejection” (AR) is defined for biopsy samples with a Banff tubulitis score (t) of less than or equal to 1 and an interstitial infiltrate score of less than or equal to 0; “Stable” (“STA”)/“no-AR” is defined for biopsy samples displaying an absence of AR (no-AR) or any other substantial pathology; and “Other” is defined for samples displaying an absence of Banff-graded AR, but either meet the Banff criteria for chronic allograft injury, chronic calcineurin inhibitor toxicity, BK viral infection, or other graft injury.

### Evaluation of Gene Expression in Biological Samples

Biological samples taken from a renal allograft recipient can be used to evaluate the level of genes which are differentially expressed in individuals undergoing an AR response. Various techniques of measuring gene expression are known to one of skill in the art. One non-limiting method is to extract RNA from the collected biological sample and to synthesize cDNA. The cDNA can then be amplified using primers or labeled primers specific for the target genes (i.e., genes which are differentially expressed in individuals undergoing an AR response) and subsequently analyzed using quantitative polymerase chain reaction (qPCR). qPCR platforms such as BioMark (Fluidigm, South San Francisco, Calif.) or ABI viia7 (Life Technologies, Foster City, Calif.) may be used.

In some embodiments, one of either the gene specific primers or dNTPs, preferably the dNTPs, will be labeled such that the synthesized cDNAs are labeled. By labeled is meant that the entities comprise a member of a signal producing system and are thus detectable, either directly or through combined action with one or more additional members of a signal producing system. Examples of directly detectable labels include isotopic and fluorescent moieties incorporated into, usually covalently bonded to, a nucleotide monomeric unit, e.g. dNTP or monomeric unit of the primer. Isotopic moieties or labels of interest include 32 P, 33 P, 35 S, 125 I, and the like. Fluorescent moieties or labels of interest include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. texas red, tetramethylrhodamine, eosins and erythrosins, cyanine dyes, e.g. Cy3 and Cy5, macrocyclic chelates of lanthanide ions, e.g. quantum Dye™, fluorescent energy transfer dyes, such as thiazole orange-ethidium heterodimer, TOTAB, etc. Labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody; and the like. Labeled nucleic acid can also be produced by carrying out PCR in the presence of labeled primers. U.S. Pat. No. 5,994,076 is incorporated by reference solely for its teachings of modified primers and dNTPs thereof.

Exemplary differentially expressed genes in renal allograft recipients who are undergoing an AR response are listed in Table 1. In one embodiment, a differentially expressed gene is indicated by a p-value less than or equal to 0.05, or a false discovery rate less than or equal to 5%, and can be considered significant and utilized to build prediction models. In another embodiment, a gene with an absolute fold change greater than or equal to 1.5 and a p-value less than or equal to 0.05, or a false discovery rate less than or equal to 5% can be considered significant and utilized to build prediction models. Various types of software can be used for statistical analysis. One example of such software is Partek Genomics Suite. The genes can be subjected to statistical analysis to select a robust model for detection and/or prediction of AR. Various classification models such as penalized logistic regression, support vector machine, and partial least square discriminant analysis with equal prior probability can be used. As further detailed in the Examples, Principal Component Analysis can be used to visualize raw qPCR data, ANOVA and Student T-test can detect significantly differentially expressed genes, and Shrinking Centroids can be applied to identify the genes that discriminate between AR and no-AR samples.

From the genes listed in Table 1, a subset of 17 genes was identified that can classify patients as AR or no-AR, irrespective of patient age, transplant center, RNA source, assay, cause of end-stage renal disease, co-morbidities, and/or immunosuppression usage. This 17-gene set is made up of a combination of 10 genes that were previously shown to be indicative of AR in pediatric patients (CFLAR, DUSP1, ITGAX, RNF130, PSEN1, NKTR, RYBP, NAMPT, MAPK9, and IFNGR1) 6 newly defined genes indicative of AR in adult patients (CEACAM4, RHEB, GZMK, RARA, SLC25A37, and EPOR), and Retinoid X receptor alpha (RXRA). The sequences of these genes are provided in Appendix A and B. The genes disclosed herein can be used for various methods of diagnosing AR in an individual who has received a renal allograft, for selecting patients for treatment, as well as for other uses described herein.

TABLE 1
43 Genes identified as significantly differentially altered in AR

| Gene Symbol | Ensembl ID | Entrez ID | Definition | TaqMan assay ID |
|---|---|---|---|---|
| RYBP | ENSG00000163602 | 23429 | RING1 and YY1 binding | Hs00171928_m1 |
| | | | protein | |
| RNF130 | ENSG00000113269 | 55819 | Ring finger protein 130 | Hs00218335_m1 |
| PSEN1 | ENSG00000080815 | 5663 | presenilin 1 | Hs00997789_m1 |
| NKTR | ENSG00000114857 | 4820 | natural killer-tumor | Hs00234637_m1 |
| | | | recognition sequence | |
| NAMPT | ENSG00000105835 | 10135 | Nicotinamide | Hs00237184_m1 |
| | | | phosphoribosyltransferase | |
| MAPK9 | ENSG00000050748 | 5601 | mitogen-activated protein | Hs00177102_m1 |
| | | | kinase 9 | |
| ITGAX | ENSG00000140678 | 3687 | integrin, alpha X | Hs00174217_m1 |
| | | | (complement component | |
| | | | 3 receptor 4subunit) | |
| IFNGR1 | ENSG00000027697 \| | 3459 | interferon gamma | Hs00166223_m1 |
| | LRG_66 | | receptor 1 | |
| DUSP1 | ENSG00000120129 | 1843 | dual specificity | Hs00610256_g1 |
| | | | phosphatase 1 | |
| CFLAR | ENSG00000003402 | 8837 | CASH and FADD-like | Hs00236002_m1 |
| | | | apoptosis regulator | |
| 5LC25A37 | ENSG00000147454 | 51312 | solute carrier family 25, | Hs00249769_m1 |
| | | | member 37 | |
| RXRA | ENSG00000186350 | 6256 | retinoid X receptor, alpha | Hs01067640_m1 |
| RHEB | ENSG00000106615 | 6009 | Ras homolog enriched in | Hs02858186_m1 |
| | | | brain | |
| RARA | ENSG00000131759 | 5914 | retinoic acid receptor, | Hs00940446_m1 |
| | | | alpha | |
| GZMK | ENSG00000113088 | 3003 | granzyme K (granzyme 3; | Hs00157875_m1 |
| | | | tryptase II) | |
| EPOR | ENSG00000187266 | 2057 | erythropoietin receptor | Hs00959427_m1 |
| CEACAM4 | ENSG00000105352 | 1089 | carcinoembryonic | Hs00156509_m1 |
| | | | antigen-related cell | |
| | | | adhesion molecule 4 | |
| NFE2 | ENSG00000123405 | 4778 | nuclear factor (erythroid- | Hs00232351_m1 |
| | | | derived 2), 45 kDa | |
| MPP1 | ENSG00000130830 | 4354 | membrane protein, | Hs00609971_m1 |
| | | | palmitoylated 1, 55 kDa | |
| MAP2K3 | ENSG00000034152 | 5606 | mitogen-activated protein | Hs00177127_m1 |
| | | | kinase kinase 3 | |
| IL2RB | ENSG00000100385 | 3560 | interleukin 2 receptor, | Hs01081697_m1 |
| | | | beta | |
| FOXP3 | ENSG00000049768 \| | 50943 | forkhead box P3 | Hs00203958_m1 |
| | LRG_62 | | | |
| CXCL10 | ENSG00000169245 | 3627 | chemokine (C-X-C motif) | Hs00171042_m1 |
| | | | ligand 10 | |
| C1orf38 | ENSG00000130775 | 9473 | chromosome 1 open | Hs00985482_m1 |
| | | | reading frame 38 | |
| GZMB | ENSG00000100453 | 3002 | Granzyme B | Hs00188051_m1 |
| ABTB1 | ENSG00000114626 | 80325 | ankyrin repeat and BTB | Hs00261395_m1 |
| | | | (P02) domain containing 1 | |
| IL7R | ENSG00000168685 \| | 3575 | interleukin 7 receptor | Hs00233682_m1 |
| | LRG_74 | | | |
| STAT3 | ENS000000168610 | 6774 | signal transducer and | Hs01047580_m1 |
| | | | activator of transcription | |
| | | | 3 (acute-phase response | |
| | | | factor) | |
| YPEL3 | ENSG00000090238 | 83719 | yippee-like 3 | Hs00368883_m1 |
| | | | (Drosophila) | |
| PFN1 | ENSG00000108518 | 5216 | profilin 1 | Hs00748915 s1 |
| IL7 | ENSG00000104432 | 3574 | interleukin 7 | Hs00174202_m1 |
| PCTP | ENSG00000141179 | 58488 | phosphatidylcholine | Hs00221886_m1 |
| | | | transfer protein | |
| GBP2 | ENSG00000162645 | 2634 | guanylate binding protein | Hs00894837_m1 |
| | | | 2, interferon-inducible | |
| GBP1 | ENSG00000117228 | 2633 | guanylate binding protein | Hs00977005_m1 |
| | | | 1, interferon-inducible, | |
| | | | 67 kDa | |
| ANK1 | ENSG00000029534 | 286 | ankyrin 1, erythrocytic | Hs00986657_m1 |
| INPP5D | ENSG00000168918 | 3635 | inositol polyphosphate-5- | Hs00183290_m1 |
| | | | phosphatase, 145 kDa | |
| CHST11 | ENSG00000171310 | 50515 | Carbohydrate | Hs00218229_m1 |
| | | | (chondroitin 4) | |
| | | | sulfotransferase 11 | |
| TNFRSF1A | ENSG00000067182 \| | 7132 | tumor necrosis factor | Hs01042313_m1 |
| | LRG_193 | | receptor superfamily, | |
| | | | member 1A | |
| LYST | ENSG00000143669 | 1130 | lysosomal trafficking | Hs00915897_m1 |
| | | | regulator | |
| ADAMS | ENSG00000151651 | 101 | ADAM metallopeptidase | Hs00923282_g1 |
| | | | domain 8 | |
| RUNX3 | ENSG00000020633 | 864 | runt-related transcription | Hs00231709_m1 |
| | | | factor 3 | |
| PSMB9 | ENSG00000240065 \| | 5698 | proteasome (prosome, | Hs00544762_m1 |
| | ENSG00000239836 \| | | macropain) subunit, beta | |
| | ENSG00000243958 \| | | type, 9 (large | |
| | ENSG00000243594 \| | | multifunctional peptidase | |
| | ENSG00000243067 \| | | 2) | |
| | ENSG00000243067 \| | | | |
| | ENSG00000242711 \| | | | |
| | ENSG00000240508 \| | | | |
| | ENSG00000240118 \| | | | |
| ISG20 | ENSG00000172183 | 3669 | interferon stimulated | Hs00158122 ml |
| | | | exonuclease gene 20 kDa | |

Another non-limiting method of measuring gene expression is northern blotting. The gene expression level of genes that encode proteins can also be determined using protein quantification methods such as western blotting. Use of proteomic assays to measure the level of differentially expressed genes is also embraced herein. A person of skill in the art would know how to use standard proteomic assays in order to measure the level of gene expression.

### Reference Expression Vectors

The invention provides for the generation of reference expression vectors that are independent of age, transplant center, RNA source, assay, cause of end-stage renal disease, co-morbidities, and/or immunosuppression usage. The use of these reference expression vectors does not require the removal of batch effects that is typically required by commercial software packages such as Partek or open source software such as R.

Significant random effects on data are inferred by different transplantation centers. These random effects arise from differences in biological sample collection protocols and immunosuppressive regimens at the various transplant centers. Accordingly, individual transplant center-specific AR prediction models are more accurate than a single AR prediction model for all transplant centers.

As exemplified in the Examples and the Appendices, for a given transplant center, AR prediction models can be developed by creating a first reference expression vector for AR samples collected at that transplant center for each gene, and a second reference expression vector for no-AR samples collected at the same transplant center for each gene. The samples used to create the reference expression vector may be classified using allograft biopsies. Subsequently, the expression level of a differentially expressed gene obtained from a biological sample collected from a renal allograft recipient at the same transplant center (i.e., an “unknown” sample) can be compared to the two reference expression vectors of the AR and no-AR samples. Computer programs such as kSAS, a modified version of Lineage Profiler, can be used to assign a categorical value or score and/or a numerical value or score to each evaluated gene set that indicates the risk of AR or risk of no-AR (source code provided in Appendix C). Multiple gene set models may be used. An advantage of using multiple gene set models is that distinct values or scores are assigned for each gene set, thus minimizing the risk of a bias based on a single gene model.

In one embodiment, there are 2, 3, 4, 5, 6, 7, 8, 9, 10, 11, 12, 13, 14, 15, 16, or 17 reference expression vectors for the diagnosis of AR. In a related embodiment, there are 2, 3, 4, 5, 6, 7, 8, 9, 10, 11, 12, 13, 14, 15, 16, or 17 reference expression vectors for the diagnosis of no-AR. In one specific embodiment, there are 17 reference expression vectors for the diagnosis of AR and 17 reference expression vectors for the diagnosis of no-AR. In another specific embodiment, there are 16 reference expression vectors for the diagnosis of AR and 16 reference expression vectors for the diagnosis of no-AR. In another specific embodiment, there are 15 reference expression vectors for the diagnosis of AR and 15 reference expression vectors for the diagnosis of no-AR. In another specific embodiment, there are 12 reference expression vectors for the diagnosis of AR and 12 reference expression vectors for the diagnosis of no-AR.

In one embodiment, to generate reference expression vectors, biological samples are be collected and profiled using a 12-gene model set prior to analysis of the unknown samples. Exemplary 12-gene models are provided in Table 2. In another embodiment, biological samples are be collected and profiled using a 12-gene model set comprising BASP1, CD6, CD7, CXCL10, CXCL9, INPP5D, ISG20, LCK, NKG7, PSMB9, RUNX3, and TAP1 prior to analysis of the unknown samples. In one embodiment the 12 gene set is composed of CFLAR, PSEN1, CEACAM4, NAMPT, RHEB, GZMK, NKTR, DUSP1, RARA, ITGAX, SLC25A37, and EPOR. In one embodiment the 12 gene set is composed of CFLAR, PSEN1, CEACAM4, NAMPT, RHEB, GZMK, NKTR, DUSP1, ITGAX, SLC25A37, RXRA, and EPOR. In one embodiment the 12 gene set is composed of CFLAR, PSEN1, CEACAM4, RHEB, GZMK, NKTR, DUSP1, RARA, ITGAX, SLC25A37, RXRA, and EPOR. In one embodiment the 12 gene set is composed of CFLAR, PSEN1, CEACAM4, NAMPT, GZMK, NKTR, DUSP1, ITGAX, SLC25A37, RYBP, RXRA, and EPOR. In one embodiment the 12 gene set is composed of CFLAR, MAPK9, PSEN1, CEACAM4, GZMK, NKTR, DUSP1, RARA, SLC25A37, RYBP, RXRA, and EPOR. In one embodiment the 12 gene set is composed of CFLAR, PSEN1, CEACAM4, GZMK, NKTR, DUSP1, RARA, ITGAX, SLC25A37, RYBP, RXRA, and EPOR. In one embodiment the 12 gene set is composed of CFLAR, MAPK9, PSEN1, CEACAM4, NAMPT, GZMK, NKTR, DUSP1, ITGAX, SLC25A37, RXRA, and EPOR. In one embodiment the 12 gene set is composed of CFLAR, PSEN1, CEACAM4, NAMPT, GZMK, NKTR, DUSP1, RARA, ITGAX, SLC25A37, RYBP, and EPOR. In one embodiment the 12 gene set is composed of CFLAR, PSEN1, CEACAM4, NAMPT, GZMK, NKTR, DUSP1, RARA, ITGAX, SLC25A37, RXRA, and EPOR. In one embodiment the 12 gene set is composed of CFLAR, MAPK9, PSEN1, CEACAM4, GZMK, NKTR, DUSP1, RARA, ITGAX, SLC25A37, RXRA, and EPOR. In one embodiment the 12 gene set is composed of CFLAR, MAPK9, PSEN1, CEACAM4, GZMK, NKTR, DUSP1, RARA, ITGAX, SLC25A37, RYBP, and EPOR. In one embodiment the 12 gene set is composed of CFLAR, MAPK9, PSEN1, CEACAM4, NAMPT, GZMK, NKTR, ITGAX, SLC25A37, RYBP, RXRA, and EPOR. In one embodiment the 12 gene set is composed of CFLAR, MAPK9, PSEN1, CEACAM4, NAMPT, GZMK, NKTR, DUSP1, RARA, ITGAX, SLC25A37, and EPOR. In one embodiment the 12 gene set is composed of BASP1, CD6, CD7, CXCL10, CXCL9, INPP5D, ISG20, LCK, NKG7, PSMB9, RUNX3, and TAP1.

TABLE 2
Kidney AR prediction assay Performance - Selected
14 12-gene Models from Selected 17 genes

| | Adult | Pediatric + | |
|---|---|---|---|
| | Training- | Adult Test- | |
| Model | Set (n = 32) | Set (n = 68) | Description |
| 1 | 90.63% | 88.24% | CFLAR, PSEN1, CEACAM4, NAMPT, RHEB, GZMK, |
| | | | NKTR, DUSP1, RARA, ITGAX, SLC25A37, EPOR |
| 2 | 90.63% | 86.27% | CFLAR, PSEN1, CEACAM4, NAMPT, RHEB, GZMK, |
| | | | NKTR, DUSP1, ITGAX, SLC25A37, RXRA, EPOR |
| 3 | 90.63% | 86.27% | CFLAR, PSEN1, CEACAM4, RHEB, GZMK, NKTR, |
| | | | DUSP1, RARA, ITGAX, SLC25A37, RXRA, EPOR |
| 4 | 90.63% | 84.31% | CFLAR, PSEN1, CEACAM4, NAMPT, GZMK, NKTR, |
| | | | DUSP1, ITGAX, SLC25A37, RYBP, RXRA, EPOR |
| 5 | 90.63% | 82.35% | CFLAR, MAPK9, PSEN1, CEACAM4, GZMK, NKTR, |
| | | | DUSP1, RARA, SLC25A37, RYBP, RXRA, EPOR |
| 6 | 90.63% | 80.39% | CFLAR, PSEN1, CEACAM4, GZMK, NKTR, DUSP1, |
| | | | RARA, ITGAX, SLC25A37, RYBP, RXRA, EPOR |
| 7 | 90.63% | 80.39% | CFLAR, MAPK9, PSEN1, CEACAM4, NAMPT, GZMK, |
| | | | NKTR, DUSP1, ITGAX, SLC25A37, RXRA, EPOR |
| 8 | 90.63% | 80.39% | CFLAR, PSEN1, CEACAM4, NAMPT, GZMK, NKTR, |
| | | | DUSP1, RARA, ITGAX, SLC25A37, RYBP, EPOR |
| 9 | 90.63% | 80.39% | CFLAR, PSEN1, CEACAM4, NAMPT, GZMK, NKTR, |
| | | | DUSP1, RARA, ITGAX, SLC25A37, RXRA, EPOR |
| 10 | 90.63% | 78.43% | CFLAR, MAPK9, PSEN1, CEACAM4, GZMK, NKTR, |
| | | | DUSP1, RARA, ITGAX, SLC25A37, RXRA, EPOR |
| 11 | 90.63% | 78.43% | CFLAR, MAPK9, PSEN1, CEACAM4, GZMK, NKTR, |
| | | | DUSP1, RARA, ITGAX, SLC25A37, RYBP, EPOR |
| 12 | 90.63% | 78.43% | CFLAR, MAPK9, PSEN1, CEACAM4, NAMPT, GZMK, |
| | | | NKTR, ITGAX, SLC25A37, RYBP, RXRA, EPOR |
| 13 | 90.63% | 78.43% | CFLAR, MAPK9, PSEN1, CEACAM4, NAMPT, GZMK, |
| | | | NKTR, DUSP1, RARA, ITGAX, SLC25A37, EPOR |
| 14 | N/A | N/A | BASP1, CD6, CD7, CXCL10, CXCL9, INPP5D, ISG20, |
| | | | LCK, NKG7, PSMB9, RUNX3, and TAP1 |

After obtaining qPCR profiles for these samples, the mean expression of all AR and no-AR samples is taken separately to create a two-column reference for all genes assayed. Alternatively, the use of a pooled RNA reference instead of individual samples can be sufficient. The data are saved as a three-column reference file, with the first column containing the gene identification, the second column containing the AR reference, and third column containing the no-AR reference. Re-analysis of the original samples used for this reference can determine if significant variability among these reference samples exist due to, for example, poor classification scores between AR and no-AR samples.

In another embodiment, to generate reference expression vectors, biological samples are collected and profiled using a 17-gene model set comprising CEACAM4, CFLAR, DUSP1, ITGAX, RNF130, PSEN1, NKTR, RYBP, NAMPT, MAPK9, IFNGR1, RHEB, GZMK, RARA, SLC25A37, EPOR, and RXRA prior to analysis of the unknown samples. In some aspects, biological samples are collected and profiled using a 12-gene model set from Table 2 comprising BASP1, CD6, CD7, CXCL10, CXCL9, INPP5D, ISG20, LCK, NKG7, PSMB9, RUNX3, and TAP1. These samples serve as transplant center-specific references. After obtaining qPCR profiles for these samples, the mean expression of all AR and no-AR samples is taken separately to create a two-column reference for all genes assayed. Alternatively, the use of a pooled RNA reference instead of individual samples can be sufficient. The data are saved as a three-column reference file, with the first column containing the gene identification, the second column containing the AR reference, and third column containing the no-AR reference. Re-analysis of the original samples used for this reference can determine if significant variability among these reference samples exist due to, for example, poor classification scores between AR and no-AR samples.

In order to classify an “unknown” sample as AR or no-AR, the expression profile of the “unknown” sample is directly compared to the reference AR profile and the reference no-AR profile. The sample is classified as AR if the sample expression profile more closely matches that of the reference AR expression profile than that of the reference no-AR expression profile. A z-score can be calculated as one measure of accuracy (see Example 2). The expression profile can be assessed by evaluating the expression of mRNA can be assessed by evaluating the cDNA, reverse transcribed from the mRNA.

### Methods of Using Gene Expression for Assessing AR/No-AR in a Renal Allograft Recipient

The differentially expressed genes as described herein can be used to diagnose or aid in the diagnosis of an individual undergoing AR or who will undergo AR. The expressed genes can also be used to monitor the progression of AR, monitor the regression of AR, identify patients who should be treated for AR or continue to be treated for AR, assess efficacy of treatment for AR, identify patients who should be monitored for AR, and/or identify an individual who is not at risk of AR. The differentially expressed genes as described herein can be used to diagnose or aid in the diagnosis of an individual not undergoing AR, diagnose or aid in the diagnosis of an individual not undergoing AR, diagnose or aid in the diagnosis of the prediction of the risk that the individual will undergo AR or will not undergo AR.

A diagnostic array can be used to quantify the differentially expressed genes present in the biological samples taken from a renal allograft recipient. The array can include a DNA-coated substrate comprising a plurality of discrete, known regions on the substrate. The arrays can comprise particles, nanoparticles, beads, nanobeads, or other solid surfaces which can be porous or non-porous, and can range in size. In one embodiment, the array is a microarray chip. In another embodiment, the diagnostic array comprises beads. In a further embodiment, the diagnostic array comprises nanoparticles. In a further embodiment, the diagnostic array comprises microfluidics.

One benefit of using the differentially expressed genes as disclosed herein is that determination of AR can be done with a high level of accuracy. Accuracy can be portrayed by sensitivity (the accuracy of the AR patients correctly identified) and by specificity (the accuracy of the no-AR patients correctly identified); positive predictive value (PPV) and negative predictive value (NPV), respectively.

In the embodiments provided herein, determination of AR using the differentially expressed genes is highly accurate for the detection or prediction of AR. In the embodiments provided herein, the methods provide at least 70%, at least 75%, at least 80%, at least 85%, at least 90%, at least 95%, at least 96%, at least 97%, at least 98%, at least 99%, or 100% accuracy. Furthermore, in the embodiments provided herein, the methods provide at least 70%, at least 75%, at least 80%, at least 85%, at least 90%, at least 95%, at least 96%, at least 97%, at least 98%, at least 99%, or 100% accuracy for the detection, or prediction of AR.

In the embodiments provided herein, determination of AR using the differentially expressed genes is highly sensitive for the detection or prediction of AR. In the embodiments provided herein, the methods provide at least 70%, at least 75%, at least 80%, at least 85%, at least 90%, at least 95%, at least 96%, at least 97%, at least 98%, at least 99%, or 100% sensitivity. Furthermore, in the embodiments provided herein, the methods provide at least 70%, at least 75%, at least 80%, at least 85%, at least 90%, at least 95%, at least 96%, at least 97%, at least 98%, at least 99%, or 100% sensitivity for the detection or prediction of AR.

Furthermore, in the embodiments provided herein, analysis of AR using the differentially expressed genes is highly specific for the detection or prediction of AR. In the embodiments provided herein, the methods provide at least 70%, at least 75%, at least 80%, at least 85%, at least 90%, at least 95%, at least 96%, at least 97%, at least 98%, at least 99%, or 100% specificity. Furthermore, in the embodiments provided herein, the methods provide at least 70%, at least 75%, at least 80%, at least 85%, at least 90%, at least 95%, at least 96%, at least 97%, at least 98%, at least 99%, or 100% specificity for the detection or prediction of AR.

Moreover, in the embodiments provided herein, analysis of AR using the differentially expressed genes has a positive predictive value (PPV; the proportion of positive test results that are true positives/correct diagnoses) for the detection or prediction of AR. In the embodiments provided herein, the methods provide at least 70%, at least 75%, at least 80%, at least 85%, at least 90%, at least 95%, at least 96%, at least 97%, at least 98%, at least 99%, or 100% PPV for the detection or prediction of AR. Also, in the embodiments provided herein, analysis of AR using the differentially expressed genes has a negative predictive value (NPV; the proportion of subjects with a negative test result who are correctly diagnosed) for the detection or prediction of AR. In the embodiments provided herein, the methods provide at least 70%, at least 75%, at least 80%, at least 85%, at least 90%, at least 95%, at least 96%, at least 97%, at least 98%, at least 99%, or 100% NPV, for the detection or prediction of AR.

The analysis of biological samples from a renal allograft recipient include evaluation of combinations of 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or more, 95 or more, 96 or more, 97 or more, 98 or more, 99 or more, 100 or more, 101 or more, or even 102 differentially expressed genes disclosed herein. In some embodiments, about 1 to about 43 genes, including all iterations of integers of the number of genes within the specified range of Table 1 are measured from biological samples from a renal allograft recipient by the methods described herein. In some embodiments, about 1 to about 12 genes, including all iterations of integers of the number of genes within the specified range of Table 2 are measured from biological samples from a renal allograft recipient by the methods described herein. In some embodiments, about 1 to about 102 genes, including all iterations of integers of the number of genes within the specified range of Table 3 are measured from biological samples from a renal allograft recipient by the methods described herein.

In one embodiment, the analysis of differentially expressed genes from a renal allograft recipient comprises measuring the level of CEACAM4 and 6 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In another embodiment, the analysis of differentially expressed genes from a renal allograft recipient comprises measuring the level of CEACAM4 and 7 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In a further embodiment, the analysis of differentially expressed genes from a renal allograft recipient comprises measuring the level of CEACAM4 and 8 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In another embodiment, the analysis of differentially expressed genes from a renal allograft recipient comprises measuring the level of CEACAM4 and 9 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In still another embodiment, the analysis of differentially expressed genes from a renal allograft recipient comprises measuring the level of CEACAM4 and 10 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In a further embodiment, the analysis of differentially expressed genes from a renal allograft recipient comprises measuring the level of CEACAM4 and 11 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In another embodiment, the analysis of differentially expressed genes from a renal allograft recipient comprises measuring the level of CEACAM4 and 12 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In a further embodiment, the analysis of differentially expressed genes from a renal allograft recipient comprises measuring the level of CEACAM4 and 13 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In another embodiment, the analysis of differentially expressed genes from a renal allograft recipient comprises measuring the level of CEACAM4 and 14 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In a further embodiment, the analysis of differentially expressed genes from a renal allograft recipient comprises measuring the level of CEACAM4 and 15 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In another embodiment, the analysis of differentially expressed genes from a renal allograft recipient comprises measuring the level of BASP1, CD6, CD7, CXCL10, CXCL9, INPP5D, ISG20, LCK, NKG7, PSMB9, RUNX3, and TAP1.

In a further embodiment, the analysis of differentially expressed genes from a renal allograft recipient comprises measuring the level of a combination of 12 genes as selected from Table 2.

In a further embodiment, the analysis of differentially expressed genes from a renal allograft recipient comprises measuring the level of the genes CEACAM4, CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. This 17-gene set corrected predicts 88% of samples as AR and 95% of samples as no-AR. In some embodiments, the expression level of a total of 17 genes is measured.

In a further embodiment, the analysis of differentially expressed genes from a renal allograft recipient comprises measuring the level of the genes BASP1, CD6, CD7, CXCL10, CXCL9, INPP5D, ISG20, LCK, NKG7, PSMB9, RUNX3, and TAP1.

In another embodiment, the analysis of differentially expressed genes from a renal allograft recipient comprises measuring the level of the genes CEACAM4, CFLAR, DUSP1, ITGAX, NAMPT, NKTR, PSEN1, EPOR, GZMK, RARA, RHEB, and SLC25A37. This gene set classifies AR with 86% sensitivity and 90% specificity.

In another embodiment, the analysis of the differentially expressed genes described herein is useful for predicting chronic injury to a renal allograft. Chronic injury typically is described as a long-term loss of function in a transplanted organ, most commonly through prolonged immune responses raised against the donor organ. In one aspect, the differentially expressed genes are assessed in tissue biopsy samples from a subject. In another aspect, the measurement of the differentially expressed genes in a tissue biopsy can be carried out by immunohistochemical techniques, nucleic acid methods as described herein, or protein detection methods (e.g., western blotting) or other common gene expression methodologies known in the art. In another aspect, the levels of CEACAM4 and between 6 and 16 other genes selected from CFLAR, DUSP1, IFNGR1, ITGAX, MAPK9, NAMPT, NKTR, PSEN1, RNF130, RYBP, EPOR, GZMK, RARA, RHEB, RXRA, and SLC25A37 is measured in a tissue biopsy from an individual who has received a renal allograft for the assessment of AR. In another aspect, the levels of CEACAM4 and between 6 and 16 other genes selected from BASP1, CD6, CD7, CXCL10, CXCL9, INPP5D, ISG20, LCK, NKG7, PSMB9, RUNX3, and TAP1 is measured in a tissue biopsy from an individual who has received a renal allograft for the assessment of AR. In another aspect, the levels of about 1 to about 43 genes, including all iterations of integers of the number of genes within the specified range, from Table 1 are measured in a tissue biopsy from an individual who has received a renal allograft for the assessment of AR. In another embodiment about 1 to about 102 of the genes, including all iterations of integers of the number of genes within the specified range, from Table 3 are measured in a tissue biopsy from an individual who has received a renal allograft for the assessment of AR.

In some embodiments, an aggregated gene model is employed. That is, multiple gene sets as described above are used, with each gene set providing a categorical value or score and/or a numerical value or score. In this way, the aggregated model is not biased on a single gene set. Among patients with a high risk of AR, 91% were correctly classified as AR. Among patients with a very low risk of AR, 92% were correctly classified as no-AR.

The differentially expressed genes of the invention can also be used to identify an individual for treatment of AR. In some embodiments, this individual is monitored for the progression or regression of AR symptoms. In some embodiments, this individual is treated for AR prior to or at the onset of AR symptoms. In some embodiments, the treatment is corticosteroid therapy. In other embodiments, the treatment is administration of an anti-T-cell antibody, such as muromonab-CD3 (Orthoclone OKT3). In further embodiments, the treatment is a combination of plasma exchange and administration of anti-CD20 antibodies. In some cases, the monitoring is done to determine if the treatment should be continued or to see if the treatment is efficacious.

In some embodiments of the methods described herein, the methods have use in predicting AR response. In these methods, a subject is first monitored for AR according to the subject methods, and then treated using a protocol determined, at least in part, on the results of the monitoring. In one embodiment, the subject is monitored for the presence or absence of acute rejection according to one of the methods described herein. The subject may then be treated using a protocol whose suitability is determined using the results of the monitoring step. For example, where the subject is predicted to have an acute rejection response within the next 1 to 6 months, immunosuppressive therapy can be modulated, e.g., increased or drugs changed, as is known in the art for the treatment/prevention of acute rejection. Likewise, where the subject is predicted to be free of current and near-term acute rejection, the immunosuppressive therapy can be reduced in order to reduce the potential for drug toxicity. In some embodiments of the methods described herein, a subject is monitored for acute rejection following receipt of a graft or transplant. The subject may be screened once or serially following transplant receipt, e.g., weekly, monthly, bimonthly, half-yearly, yearly, etc. In some embodiments, the subject is monitored prior to the occurrence of an acute rejection episode. In other embodiments, the subject is monitored following the occurrence of an acute rejection episode.

In some embodiments of the methods described herein, the methods have use in altering or changing a treatment paradigm or regimen of a subject in need of treatment of AR. Exemplary non-limiting immunosuppressive therapeutics or therapeutic agents useful for the treating of a subject in need thereof comprise steroids (e.g., prednisone (Deltasone), prednisolone, methyl-prednisolone (Medrol, Solumedrol)), antibodies (e.g., muromonab-CD3 (Orthoclone-OKT3), antithymocyte immune globulin (ATGAM, Thymoglobulin), daclizumab (Zenapax), basiliximab (Simulect), Rituximab, cytomegalovirus-immune globulin (Cytogam), immune globulin (Polygam)), calcineurin inhibitors (e.g., cyclosporine (Sandimmune), tacrolimus (Prograf)), antiproliferatives (e.g., mycophenolate mofetil (Cellcept), azathioprine (Imuran)), TOR inhibitors (e.g., rapamycin (Rapamune, sirolimus), everolimus (Certican)), or a combination therapy thereof.

In some embodiments, wherein a subject is identified as not having an AR using the methods described herein, the subject can remain on an immunosuppressive standard of care maintenance therapy comprising the administration of an antiproliferative agent (e.g., mycophenolate mofetil and/or azathioprine), a calcineurin inhibitor (e.g., cyclosporine and/or tacrolimus), steroids (e.g., prednisone, prednisolone, and/or methyl prednisolone) or a combination thereof. For example, a subject identified as not having an AR using the methods described herein can be placed on a maintenance therapy comprising the administration of a low dose of prednisone (e.g., about 0.1 mg·kg⁻¹·d⁻¹ to about 1 mg·kg⁻¹·d⁻¹), a low dose of cyclosporine (e.g., about 4 mg·kg⁻¹·d⁻¹ to about 8 mg·kg⁻¹·d⁻¹), and a low dose of mycophenolate (e.g., about 1-1.5 g twice daily). In another example, a subject identified as not having an AR using the methods described herein can be taken off of steroid therapy and placed on a maintenance therapy comprising the administration of a low dose of cyclosporine (e.g., about 4 mg·kg⁻¹·d⁻¹ to about 8 mg·kg⁻¹·d⁻¹), and a low dose of mycophenolate (e.g., about 1-1.5 g twice daily). In another example, a subject identified as not having an AR using the methods described herein can be removed from all immunosuppressive therapeutics described herein.

In some embodiments, wherein a subject is identified as having an AR using the methods described herein, the subject may be placed on a rescue therapy or increase in immunosuppressive agents comprising the administration of a high dose of a steroid (e.g., prednisone, prednisolone, and/or methyl prednisolone), a high dose of a polyclonal or monoclonal antibody (e.g., muromonab-CD3 (OKT3), antithymocyte immune globulin, daclizumab, Rituximab, basiliximab, cytomegalovirus-immune globulin, and/or immune globulin), a high dose of an antiproliferative agent (e.g., mycophenolate mofetil and/or azathioprine), or a combination thereof.

In some embodiments, the course of therapy wherein a subject is identified as not having an AR or is identified as having an AR using the methods described herein is dependent upon the time after transplantation and the severity of rejection, treating physician, and the transplantation center.

Therefore, using the differentially expressed genes of the invention and the methodology described herein, one of skill in the art can diagnose AR in a renal allograft recipient, diagnose no-AR in a renal allograft recipient, aid in the diagnosis of AR, aid in the diagnosis of the risk of AR, monitor the progression of AR, monitor the regression of AR, identify an individual who should be treated for AR or continue to be treated for AR, assess efficacy of treatment for AR, and/or identify individuals who should be monitored for AR symptoms.

In some embodiments, the differentially expressed genes of the invention and the methodology described herein, can be used for the stratification or identification of antibody mediated AR. In other embodiments, the differentially expressed genes of the invention and the methodology described herein, can be used for the stratification or identification of T-cell mediated AR. The genes provided herein are useful for identification of B-cell or T-cell mediated AR in some aspects because they are either expressed on B cells or are expressed on T-cells or are known markers of activated T-cells.

### Kits for the Diagnosis, Detection, or Prediction of AR

The invention further provides for assay kits for the diagnosis, detection, and prediction of AR. The kit comprises a gene expression evaluation element for measuring the level of differentially expressed genes associated with AR in a biological sample from an individual who has received a renal allograft. In some embodiments, the kit comprises reagents for measuring the level of differentially expressed genes of interest in the biological sample. In some embodiments, the kit comprises a composition comprising one or more solid surfaces for the measurement of the differentially expressed genes of interest in the biological sample. In one embodiment, the solid surface comprises a microarray chip. In another embodiment, the solid surface comprises a bead. In a further embodiment, the solid surface comprises a nanoparticle. In one embodiment, the kit comprises a composition comprising one or more solid surfaces for the measurement of CEACAM4 and at least 6, 7, 8, 9, 10, or 11 other genes selected from CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In some embodiments, the expression level of a total of 17 genes is measured.

The kit further comprises a reference standard element for use in diagnosing AR in an individual who has received a renal allograft. In some embodiments, the reference standard element comprises a single reference expression vector from AR samples for each differentially expressed gene obtained from renal allograft recipients from a single transplant center or across transplant centers. In some embodiments, the reference standard element comprises a single reference expression vector from no-AR samples for each differentially expressed gene obtained from renal allograft recipients from a single transplant center or across transplant centers. The reference standard element is used for comparison to the gene expression from a renal allograft recipient in order to diagnose the recipient with AR.

In some embodiments, the comparison is performed by a computer. In other embodiments, the comparison is performed by an individual. In one embodiment, the comparison is performed by a physician. The reference standards for each transplant center can be prepared as described above.

In some embodiments a computer is configured to output to a user at least one of: a prediction of an onset of an AR response, a diagnosis of an AR response, and a characterization of an AR response in the subject, wherein the output is determined by comparing the gene expression result of 7, 8, 9, 10, 11, 12, 13, 14, 15, 16, or 17 genes to a control reference expression profile.

The kit also comprises instructions for the use of the assay.

### Systems for the Diagnosis, Detection, or Prediction of AR

The invention further provides for systems for the diagnosis, detection, and prediction of AR. The system comprises a gene expression evaluation element for measuring the level of differentially expressed genes associated with AR in a biological sample from an individual who has received a renal allograft. In one embodiment, the system comprises a microarray chip. In another embodiment, the system comprises a bead. In a further embodiment, the system comprises a nanoparticle. In various embodiments, the system comprises a gene expression evaluation element for the measurement of CEACAM4 and at least 6, 7, 8, 9, 10, 11, 12, 13, 14, 15, or 16 other genes selected from CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In some embodiments, the expression level of a total of 17 genes is measured.

In certain embodiments the gene expression evaluation element comprises a comprises a labeled gene primer or a labeled probe designed to selectively amplify CEACAM4 and the at least 6, 7, 8, 9, 10, 11, 12, 13, 14, 15, or 16 other genes selected from CFLAR, DUSP1, ITGAX, RNF130, PSEN1, NKTR, RYBP, NAMPT, MAPK9, IFNGR1, RHEB, GZMK, RARA, SLC25A37, EPOR, and RXRA to produce a gene expression result. In some embodiments the label is non-naturally occurring. In other embodiments the gene primer or probe is covalently modified to comprise the label. In related embodiments the label can be selected from the group consisting of a fluorophore or a radioactive label.

The system further comprises a reference standard element for assessing AR in an individual who has received a renal allograft. In some embodiments, the reference standard element comprises a single reference expression vector from AR samples for each differentially expressed gene obtained from renal allograft recipients from a single transplant center. In some embodiments, the reference standard element comprises a single reference expression vector from no-AR samples for each differentially expressed gene obtained from renal allograft recipients from a single transplant center. The reference standard element is used for comparison to the gene expression from a renal allograft recipient in order to diagnose the recipient with AR. In some embodiments, the comparison is performed by a computer. In other embodiments, the comparison is performed by an individual. In one embodiment, the comparison is performed by a physician. The reference standards for each transplant center can be prepared as described above.

### Compositions for the Diagnosis, Detection, or Prediction of AR

The present invention provides for compositions comprising one or more solid surfaces for measuring the level of differentially expressed genes associated with AR in a biological sample from an individual who has received a renal allograft. In some embodiments, the composition is an article of manufacture. In one embodiment, the article of manufacture comprises a reference standard for measuring the level of differentially expressed genes in a biological sample from an individual who has received a renal allograft. In some embodiments, the solid surfaces provide for the attachment of cDNA of the differentially expressed genes. In other embodiments, the solid surfaces provide for the attachment of primers or labeled primers for amplification of the differentially expressed genes. In certain embodiments, the solid surface allows measurement of at least 1, 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or more, 95 or more, 96 or more, 97 or more, 98 or more, 99 or more, 100 or more, 101 or more, or even 102 genes disclosed herein. In one embodiment about 1 to about 43 genes, including all iterations of integers of the number of genes within the specified range, from Table 1 are measured in a biological sample from an individual who has received a renal allograft for the assessment of AR. In another embodiment about 1 to about 102 of the genes, including all iterations of integers of the number of genes within the specified range, from Table 3 are measured in a biological sample from an individual who has received a renal allograft for the assessment of AR. In another embodiment, a minimum of 7 genes is measured for assessment of AR. In another embodiment, a maximum of 17 genes is measured for assessment of AR.

In one specific embodiment, the invention provides a composition which includes one or more solid surfaces for measurement the gene expression level of CEACAM4 and 6 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In another embodiment, the composition includes one or more solid surfaces for measuring the gene expression level of CEACAM4 and 7 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In a further embodiment, the composition includes one or more solid surfaces for measuring the gene expression level of CEACAM4 and 8 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In another embodiment, the composition includes one or more solid surfaces for measuring the gene expression level of CEACAM4 and 9 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In still another embodiment, the composition includes one or more solid surfaces for measuring the gene expression level of CEACAM4 and 10 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In a further embodiment, the composition includes one or more solid surfaces for measuring the gene expression level of CEACAM4 and 11 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In another embodiment, the composition includes one or more solid surfaces for measuring the gene expression level of CEACAM4 and 12 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In a further embodiment, the composition includes one or more solid surfaces for measuring the gene expression level of CEACAM4 and 13 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In another embodiment, the composition includes one or more solid surfaces for measuring the gene expression level of CEACAM4 and 14 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In a further embodiment, the composition includes one or more solid surfaces for measuring the gene expression level of CEACAM4 and 15 genes selected from the group consisting of CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In a further embodiment, the composition includes one or more solid surfaces for measuring the gene expression level of CEACAM4, CFLAR, DUSP1, ITGAX, RNF130, PSEN1, NKTR, RYBP, NAMPT, MAPK9, IFNGR1, RHEB, GZMK, RARA, SLC25A37, EPOR, and RXRA. In some embodiments, the expression level of a total of 17 genes is measured. In another embodiment, the composition includes one or more solid surfaces for measuring the gene expression level of CEACAM4, CFLAR, DUSP1, ITGAX, NAMPT, NKTR, PSEN1, EPOR, GZMK, RARA, RHEB, and SLC25A37.

TABLE 3
102 Commonly Regulated Genes in Solid Organ Transplant Acute Rejection

| Gene Symbol | Ensembl ID | Entrez ID | Definition |
|---|---|---|---|
| AIF1 | ENSG00000206428 | 199 | allograft inflammatory factor 1 |
| F13A1 | ENSG00000124491 | 2162 | coagulation factor XIII, A1 |
| | | | polypeptide |
| MMP9 | ENSG00000100985 | 4318 | matrix metallopeptidase 9 |
| | | | (gelatinase B, 92 kDa gelatinase, |
| | | | 92 kDa type IV collagenase) |
| NELL2 | ENSG00000184613 | 4753 | NEL-like 2 (chicken) |
| CD53 | ENSG00000143119 | 963 | CD53 molecule |
| CXCL10 | ENSG00000169245 | 3627 | chemokine (C-X-C motif) ligand 10 |
| ISG20 | ENSG00000172183 | 3669 | interferon stimulated exonuclease |
| | | | gene 20 kDa |
| CD48 | ENSG00000117091 | 962 | CD48 molecule |
| BATF | ENSG00000156127 | 10538 | basic leucine zipper transcription |
| | | | factor, ATF-like |
| DDX11 | ENSG00000013573 | 1663 | DEAD/H (Asp-Glu-Ala-Asp/His) |
| | | | box polypeptide 11 (CHL1-like |
| | | | helicase homolog, S. cerevisiae) |
| CD44 | ENSG00000026508 | 960 | CD44 molecule (Indian blood group) |
| DDX23 | ENSG00000174243 | 9416 | DEAD (Asp-Glu-Ala-Asp) box |
| | | | polypeptide 23 |
| IL15RA | ENSG00000134470 | 3601 | interleukin 15 receptor, alpha |
| ADAM8 | ENSG00000151651 | 101 | ADAM metallopeptidase domain 8 |
| RAB27A | ENSG00000069974 | 5873 | RAB27A, member RAS oncogene |
| | | | family |
| CD3D | ENSG00000167286 | 915 | CD3d molecule, delta (CD3-TCR |
| | | | complex) |
| HLA-A | ENSG00000206503 | 3105 | major histocompatibility complex, |
| | | | class I, A |
| TNFRSF14 | ENSG00000157873 | 8764 | tumor necrosis factor receptor |
| | | | superfamily, member 14 |
| | | | (herpesvirus entry mediator) |
| BASP1 | ENSG00000176788 | 10409 | brain abundant, membrane attached |
| | | | signal protein 1 |
| HLA-E | ENSG00000204592 | 3133 | major histocompatibility complex, |
| | | | class I, E |
| HLA-G | ENSG00000204632 | 3135 | major histocompatibility complex, |
| | | | class I, G |
| HLA-F | ENSG00000206509 | 3134 | major histocompatibility complex, |
| | | | class I, F |
| ARPC1B | ENSG00000130429 | 10095 | actin related protein 2/3 complex, |
| | | | subunit 1B, 41 kDa; similar to Actin- |
| | | | related protein 2/3 complex subunit |
| | | | 1B (ARP2/3 complex 41 kDa |
| | | | subunit) (p41-ARC) |
| KRT17 | ENSG00000186831 | 729682 | keratin 17; keratin 17 pseudogene 3 |
| ADAMTS3 | ENSG00000156140 | 9508 | ADAM metallopeptidase with |
| | | | thrombospondin type 1 motif, 3 |
| BTN3A2 | ENSG00000186470 | 11118 | butyrophilin, subfamily 3, member |
| | | | A2 |
| TNFAIP2 | ENSG00000185215 | 7127 | tumor necrosis factor, alpha-induced |
| | | | protein 2 |
| GBP2 | ENSG00000162645 | 2634 | guanylate binding protein 2, |
| | | | interferon-inducible |
| IFITM3 | ENSG00000142089 | 10410 | interferon induced transmembrane |
| | | | protein 3 (1-8U) |
| STK10 | ENSG00000072786 | 6793 | serine/threonine kinase 10 |
| MAP4K1 | ENSG00000104814 | 11184 | mitogen-activated protein kinase |
| | | | kinase kinase kinase 1 |
| ITGB2 | ENSG00000160255 | 3689 | integrin, beta 2 (complement |
| | | | component 3 receptor 3 and 4 |
| | | | subunit) |
| PTPRCAP | ENSG00000213402 | 5790 | protein tyrosine phosphatase, |
| | | | receptor type, C-associated protein |
| MDK | ENSG00000110492 | 4192 | midkine (neurite growth-promoting |
| | | | factor 2) |
| SERPINH1 | ENSG00000149257 | 871 | serpin peptidase inhibitor, clade H |
| | | | (heat shock protein 47), member 1, |
| | | | (collagen binding protein 1) |
| ITGB7 | ENSG00000139626 | 3695 | integrin, beta 7 |
| ZAP70 | ENSG00000115085 | 7535 | zeta-chain (TCR) associated protein |
| | | | kinase 70 kDa |
| FCER1G | ENSG00000158869 | 2207 | Fc fragment of IgE, high affinity I, |
| | | | receptor for; gamma polypeptide |
| RUNX3 | ENSG00000020633 | 864 | runt-related transcription factor 3 |
| ARHGDIB | ENSG00000111348 | 397 | Rho GDP dissociation inhibitor |
| | | | (GDI) beta |
| CDC20 | ENSG00000117399 | 991 | cell division cycle 20 homolog |
| | | | (S. cerevisiae) |
| AIM2 | ENSG00000163568 | 9447 | absent in melanoma 2 |
| LGALS9 | ENSG00000168961 | 3965 | lectin, galactoside-binding, soluble, |
| | | | 9 |
| PSMB9 | ENSG00000206232 | 5698 | proteasome (prosome, macropain) |
| | | | subunit, beta type, 9 (large |
| | | | multifunctional peptidase 2) |
| ATF5 | ENSG00000169136 | 22809 | activating transcription factor 5 |
| IKBKE | ENSG00000143466 | 9641 | inhibitor of kappa light polypeptide |
| | | | gene enhancer in B-cells, kinase |
| | | | epsilon |
| CORO1A | ENSG00000102879 | 11151 | coronin, actin binding protein, 1A |
| BBC3 | ENSG00000105327 | 27113 | BCL2 binding component 3 |
| UCP2 | ENSG00000175567 | 7351 | uncoupling protein 2 (mitochondrial, |
| | | | proton carrier) |
| MARCKS | ENSG00000155130 | 4082 | myristoylated alanine-rich protein |
| | | | kinase C substrate |
| NKG7 | ENSG00000105374 | 4818 | natural killer cell group 7 sequence |
| NNMT | ENSG00000166741 | 4837 | nicotinamide N-methyltransferase |
| CD8A | ENSG00000153563 | 925 | CD8a molecule |
| HLA-DMA | ENSG00000204257 | 3108 | major histocompatibility complex, |
| | | | class II, DM alpha |
| ARHGAP4 | ENSG00000089820 | 393 | Rho GTPase activating protein 4 |
| CASP4 | ENSG00000196954 | 837 | caspase 4, apoptosis-related cysteine |
| | | | peptidase |
| HCP5 | ENSG00000206337 | 10866 | HLA complex P5 |
| MAN2B1 | ENSG00000104774 | 4125 | mannosidase, alpha, class 2B, |
| | | | member 1 |
| PLCB2 | ENSG00000137841 | 5330 | phospholipase C, beta 2 |
| GZMA | ENSG00000145649 | 3001 | granzyme A (granzyme 1, cytotoxic |
| | | | T-lymphocyte-associated serine |
| | | | esterase 3) |
| LEF1 | ENSG00000138795 | 51176 | lymphoid enhancer-binding factor 1 |
| MCM5 | ENSG00000100297 | 4174 | minichromosome maintenance |
| | | | complex component 5 |
| SH2D2A | ENSG00000027869 | 9047 | SH2 domain protein 2A |
| PRKD2 | ENSG00000105287 | 25865 | protein kinase D2 |
| TNFRSF9 | ENSG00000049249 | 3604 | tumor necrosis factor receptor |
| | | | superfamily, member 9 |
| GZMK | ENSG00000113088 | 3003 | granzyme K (granzyme 3; tryptase |
| | | | II) |
| PSME1 | ENSG00000092010 | 5720 | proteasome (prosome, macropain) |
| | | | activator subunit 1 (PA28 alpha) |
| LCK | ENSG00000182866 | 3932 | lymphocyte-specific protein tyrosine |
| | | | kinase |
| DDB2 | ENSG00000134574 | 1643 | damage-specific DNA binding |
| | | | protein 2, 48 kDa |
| ADAM19 | ENSG00000135074 | 8728 | ADAM metallopeptidase domain 19 |
| | | | (meltrin beta) |
| MAP3K11 | ENSG00000173327 | 4296 | mitogen-activated protein kinase |
| | | | kinase kinase 11 |
| HLA-DQB1 | ENSG00000179344 | 3119 | major histocompatibility complex, |
| | | | class II, DQ beta 1; similar to major |
| | | | histocompatibility complex, class II, |
| | | | DQ beta 1 |
| TNF | ENSG00000206439 | 7124 | tumor necrosis factor (TNF |
| | | | superfamily, member 2) |
| FOXM1 | ENSG00000111206 | 2305 | forkhead box M1 |
| PML | ENSG00000140464 | 652346 | promyelocytic leukemia; similar to |
| | | | promyelocytic leukemia protein |
| | | | isoform 1 |
| CXCL9 | ENSG00000138755 | 4283 | chemokine (C-X-C motif) ligand 9 |
| POLR2A | ENSG00000181222 | 5430 | polymerase (RNA) II (DNA |
| | | | directed) polypeptide A, 220 kDa |
| TNFRSF1A | ENSG00000067182 | 7132 | tumor necrosis factor receptor |
| | | | superfamily, member 1A |
| RGS10 | ENSG00000148908 | 6001 | regulator of G-protein signaling 10 |
| TNFRSF1B | ENSG00000028137 | 7133 | tumor necrosis factor receptor |
| | | | superfamily, member 1B |
| NUP210 | ENSG00000132182 | 23225 | nucleoporin 210 kDa |
| IL10RA | ENSG00000110324 | 3587 | interleukin 10 receptor, alpha |
| TAP1 | ENSG00000206233 | 6890 | transporter 1, ATP-binding cassette, |
| | | | sub-family B (MDR/TAP) |
| CD2 | ENSG00000116824 | 914 | CD2 molecule |
| INPP5D | ENSG00000168918 | 3635 | inositol polyphosphate-5- |
| | | | phosphatase, 145 kDa |
| CD6 | ENSG00000013725 | 923 | CD6 molecule |
| CD7 | ENSG00000173762 | 924 | CD7 molecule |
| PTPRC | ENSG00000081237 | 5788 | protein tyrosine phosphatase, |
| | | | receptor type, C |
| IL2RB | ENSG00000100385 | 3560 | interleukin 2 receptor, beta |
| PLEK | ENSG00000115956 | 5341 | pleckstrin |
| BIRC5 | ENSG00000089685 | 332 | baculoviral IAP repeat-containing 5 |
| FZD2 | ENSG00000180340 | 2535 | frizzled homolog 2 (Drosophila) |
| STAT1 | ENSG00000115415 | 6772 | signal transducer and activator of |
| | | | transcription 1, 91 kDa |
| CCL13 | ENSG00000181374 | 6357 | chemokine (C-C motif) ligand 13 |
| IRF5 | ENSG00000128604 | 3663 | interferon regulatory factor 5 |
| STAB1 | ENSG00000010327 | 23166 | stabilin 1 |
| IRF1 | ENSG00000125347 | 3659 | interferon regulatory factor 1 |
| IRF3 | ENSG00000126456 | 3661 | interferon regulatory factor 3 |
| IRF4 | ENSG00000137265 | 3662 | interferon regulatory factor 4 |
| CD14 | ENSG00000170458 | 929 | CD14 molecule |
| CLORF38 | | | Chromosome 1 open reading frame |
| | | | 38 |
| VAMP5 | ENSG00000168899 | 10791 | vesicle-associated membrane protein |
| | | | 5 |

### Software for Correlation Based Algorithms for Classification of AR and No-AR

The correlation-based analyses described herein can be performed in AltAnalyze version 2.0.8 or later. LineageProfiler is available through a graphical user interface in the open-source software AltAnalyze (http://code.google.com/p/altanalyze/downloads, version 2.0.8 or higher) and as standalone python script (https://github.com/nsalomonis/LineageProfilerIterate). AltAnalyze can be downloaded from http://www.altanalyze.org, extracted to a hard drive, and installed with the latest human database when prompted (currently EnsMart65) following the initial launch. Alternatively, LineageProfiler functions can be performed using a command-line version of this software along with options for gene model discovery available at https://github.com/nsalomonis/LineageProfilerIterate. Instructions on running the standalone graphical user interface version of LineageProfiler and the command-line versions are described at http://code.google.com/p/altanalyze/wiki/SampleClassification. The source code for LineageProfiler was modified for use in the embodiments described herein, resulting in LineageProfiler Iterate. As used herein, LineageProfiler Iterate, modified LineageProfiler, and kSAS are used interchangeably. The source code for kSAS, is provided in Appendix C. This software can be used to classify quantitative expression values for a given set of samples as belonging to a particular disease class, phenotype, or treatment category. In brief, the algorithm does this by correlating an input set of expression values for a given sample to 2 or more reference conditions. Rather than correlating the sample with the references directly, a subset of genes can be selected from a model file, which has been previously identified to produce a high degree of predictive success using samples belonging to known classes. The algorithm can also be applied to new data to discover alternative or new gene models.

The following examples are provided for illustrative purposes. These are intended to show certain aspects and embodiments of the present invention but are not intended to limit the invention in any manner.

### EXAMPLES

### Example 1: Study Design for Development of Compositions and Methods for Assessing Acute Rejection in Renal Transplantation

The Assessment of Acute Rejection in Renal Transplantation (AART) Study was designed in a collaborative effort in 8 renal transplant centers worldwide and utilized 558 peripheral blood (PB) samples from 438 adult and pediatric renal transplant patients for developing a simple blood QPCR test for acute rejection (AR) diagnosis and prediction in recipients of diverse ages, on diverse immunosuppression, and subject to Transplant Center specific protocols.

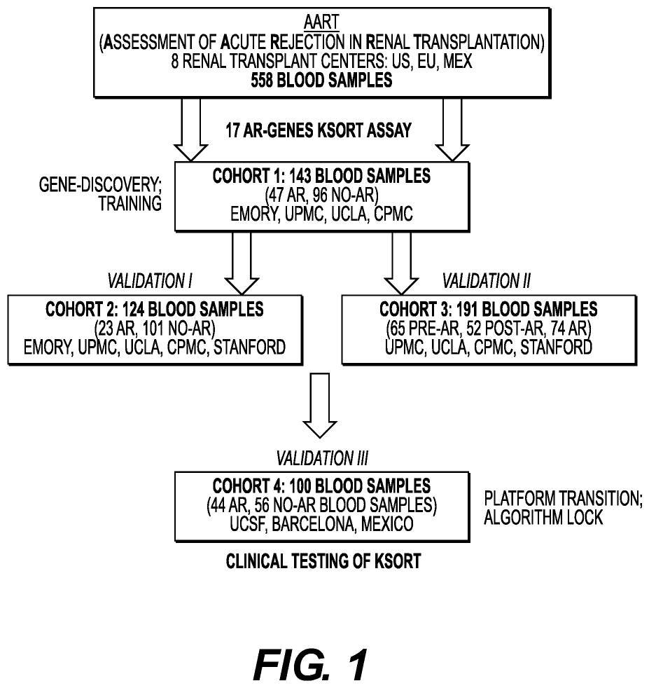

FIG. 1 describes the Assessment of Acute Rejection in Renal Transplantation (AART) Study Design in 438 unique adult/pediatric renal transplant patients from 8 transplant centers worldwide: Emory, UCLA, UPMC, CPMC, UCSF, and Barcelona contributed adult-, Mexico, and Stanford pediatric samples. For AR QPCR analysis, samples were divided into 4 Cohorts: Cohort 1 n=143 adult samples for gene modeling; Cohort 2 n=124 adult/pediatric samples for independent AR validation; Cohort 3 n=191 adult/pediatric samples for AR prediction; Cohort 4: n=100 adult/pediatric samples for final AR assay lock and clinical translation.

Blood samples were collected from transplant recipients cross-sectionally during clinical follow-up visits and were matched with a contemporaneous kidney allograft biopsy. Centers that participated in the AART study were Stanford University (Stanford; n=162 pediatric samples); Laboratorio de Investigacion en Nefrologia, Hospital Infantil de Mexico (Mex; n=23 pediatric samples); Emory University, Atlanta, Ga., (Emory, n=43 adult samples); University of California Los Angeles, Los Angeles, Calif., (UCLA, n=105 adult samples); University of Pittsburgh, Pittsburgh, Pa., (UPMC, =132 adult samples); California Pacific Medical Center, San Francisco, Calif. (CPMC, n=37 adult samples); University of California San Francisco, San Francisco, Calif., (UCSF, n=40 adult samples); Bellvitage University Hospital, Barcelona, Spain (Barcelona, n=16 samples). Samples were split into a training-set of 143 AR and No-AR adult samples (Cohort I) for gene selection and model training, into a first validation set of 124 AR and No-AR adult (>21 years) and pediatric (<21 years) samples (Cohort 2) for validation of genes for AR detection, and into a second prospective validation set of 191 adult and pediatric samples serially collected up to 6 months prior and after the rejection biopsy (Cohort 3) for evaluation of AR prediction. Blood samples composing these 3 Cohorts were simultaneously measured on the microfluidic high throughput Fluidigm QPCR platform (Biomark, Fluidigm Inc., San Francisco, Calif.) for a total of 43 genes. The final kidney AR prediction assay of 17 genes for non-invasive detection of AR was locked in an independent validation set of 100 adult and pediatric samples (Cohort 4) on the ABI QPCR platform with the development of a novel mathematical algorithm (kSAS) (FIG. 1—Study Design, and Table 4, Table 5, Patient Demographics).

TABLE 4
Demographics of 438 unique Patients

| | Stanford | Mexico | UCLA | Emory | UPMC | CMPC | UCSF | Barcelona |
|---|---|---|---|---|---|---|---|---|
| Parameters | (P) | (P) | (A) | (A) | (A) | (A) | (A) | (A) |
| Total samples | 102 (54) | 23 (15) | 59 (37) | 43 (18) | 120 (48) | 35 (4) | 40 (14) | 16 (8) |
| (Bx confirmed | | | | | | | | |
| AR) | | | | | | | | |
| Donor age | 33.3 ± 14.6 | 34.3 ± 6 | 41.4 ± 12.4 | 36.2 ± 12 | 49.8 ± 12.8 | 48 ± 13 | 43 ± 12.8 | 45.2 ± 11.3 |
| (yrs., | | | | | | | | |
| mean ± SD) | | | | | | | | |
| Donor gender | 47 | 44 | 42.7 | 38 | 37.6 | 46 | 45 | 61.7 |
| (% male) | | | | | | | | |
| Transplant | 37.4 | 28 | 42.7 | 70.4 | 17 | 43% | 60 | 91.4 |
| Type | | | | | | | | |
| (% deceased) | | | | | | | | |
| Recipient age | 12.5 ± 5 | 14.7 ± 6 | 44.9 ± 12.2 | 46 ± 15 | 47 ± 18 | 51.6 ± 16 | 53.4 ± 11 | 47 ± 10.8 |
| (yrs., | | | | | | | | |
| mean ± SD) | | | | | | | | |
| Recipient | 62 | 65 | 74.8 | 62 | 61.4 | 60.7% | 56.5% | 55% |
| gender | | | | | | | | |
| (% male) | | | | | | | | |
| HLA | 3.1 ± 1.5 | 3.3 ± 1.1 | 3.8 ± 1.5 | 3.7 ± 1.5 | 3 ± 1.7 | 3.1 ± 2 | 3.4 ± 1.8 | 3.9 ± 0.75 |
| mismatch | | | | | | | | |
| (x/6, | | | | | | | | |
| mean ± SD) | | | | | | | | |
| PRA >20% | 3 | 0 | 10 | 16.7 | 11.4 | 22.7% | 8 | 6.3 |
| (% Patients) | | | | | | | | |
| Induction | Dac | Thymo | Thymo | Thymo | Anti- | Thymo | Thymo | Thymo |
| therapy | | | | | CD52 | | | |
| Primary IS | CNI/ | CNI/ | CNI/ | CNI/ | CNI/ | CNI/ | CNI/ | CNI/ |
| | MMF/±CS | MMF/±CS | MMF/CS | MMF/CS | MMF | MMF/CS | MMF/CS | MMF/CS |
| Blood | PAXgene | PAXgene | PBMC | PBMC | PBL | PAXgene | PBMC | PAXgene |
| collection | | | | | | | | |
| method | | | | | | | | |
| Centralized | Yes | No | No | No | No | No | No | No |
| SOP | | | | | | | | |
| Centralized | Yes | No | No | Yes | No | Yes | Yes | No |
| RNA | | | | | | | | |
| Extraction | | | | | | | | |

HLA = human leukocyte antigen; PRA = panel reactive antibody; P = Pediatric; A = Adult; UPMC = University of Pittsburgh Medical Center; UCLA = University of California Los Angeles; CPMC = California Pacific Medical Center, Stanford U = Stanford University, Emory U = Emory University; CNI = Calcineurin inhibitor, DAC = Daclizumab, Thymo = Thymoglobulin, MMF = Mycophenolate mofetil; CS = Corticosteroids.

TABLE 5
Patient and sample demographics of the 659 unique pediatric (n = 293) and adult (n = 366) samples used for validation of a gene-set.

| | Stanford | SNS | Mexico | UCLA | Emory | UPMC | CMPC | UCSF | Barcelona |
|---|---|---|---|---|---|---|---|---|---|
| Parameters | (Peds) | (Peds) | (Peds) | (Adult) | (Adult) | (Adult) | (Adult) | (Adult) | (Adult) |
| Total samples | 93 (22) | 177 (22) | 23 (15) | 109 (32) | 60 (17) | 92 (28) | 53 (4) | 36 (14) | 16 (8) |
| (biopsy-confirmed | | | | | | | | | |
| AR) | | | | | | | | | |
| Donor age | 33.3 ± 14.6 | 29.3 ± 10.4 | | 41.4 ± 12.4 | 36.2 ± 12 | 49.8 ± 12.8 | | | 45.2 ± 11.3 |
| (yrs, mean ± SD) | | | | | | | | | |
| Donor gender | 47 | 61 | | 42.7 | 38 | 37.6 | | | 61.7 |
| (% male) | | | | | | | | | |
| Transplant Type | 37.4 | 53 | | 42.7 | 70.4 | 9 | | | 91.4 |
| (% deceased) | | | | | | | | | |
| Recipient age | 12.5 ± 5 | 11.6 ± 6 | | 44.9 ± 12.2 | 46 ± 15 | 47 ± 18 | | | 47 ± 10.8 |
| (yrs, mean ± SD) | | | | | | | | | |
| Recipient gender | 62 | 57 | | 74.8 | 62 | 61.4 | | | |
| (% male) | | | | | | | | | |
| HLA mismatch | 3.1 ± 1.5 | 3.9 ± 1.5 | | 3.8 ± 1.5 | 3.7 ± 1.5 | 3 ± 1.7 | | | 3.9 ± 0.75 |
| (x/6, mean ± SD) | | | | | | | | | |
| Induction therapy | Dac^{a} | Dac^{a} | Thymo^{b} | Thymo^{b} | Thymo^{b} | Anti-CD52 | Thymo^{b} | Thymo^{b} | Thymo^{b} |
| Primary | CNI^{c}, | CNI^{c}, | CNI^{c}, | CNI^{c}, | CNI^{c}, | CNI^{c}, | CNI^{c}, | CNI^{c}, | CNI^{c}, |
| immunosuppression | MMF^{d}, +/−CS^{e} | MMF^{d}, +/−CS^{e} | MMF^{d} | MMF^{d}, | MMF^{d}, | MMF^{d} | MMF^{d}, | MMF^{d}, | MMF^{d}, |
| agent | | | | CS^{e} | CS^{e} | | CS^{e} | CS^{e} | CS^{e} |
| Blood collection | PAXgene | PAXgene | PAXgene | PBMC | PBMC | PBL | PAXgene | PBMC | PAXgene |
| method | | | | | | | | | |
| Central lab blood | yes | yes | no | no | no | no | no | no | no |
| collection/storage/ | | | | | | | | | |
| processing SOP | | | | | | | | | |
| (vs. Center-specific | | | | | | | | | |
| SOP) | | | | | | | | | |
| RNA isolated in | yes | yes | no | no | yes | no | yes | yes | no |
| central lab | | | | | | | | | |

^{a}Dac = Daclizumab;
^{b}Thymo = Thymoglobulin;
^{c}CNI = Calcineurin Inhibitor;
^{d}MMF = Mycophenolate Mofetil;
^{e}CS = Corticosteroids

### Example 2: Blood Samples

Peripheral blood samples (n=518) that originated from unique pediatric (recipient age at transplant=0.8-21.9 years; n=200) and adult (recipient age at transplant=23-78 years; n=315) kidney transplant recipients were used for the development of a common peripheral blood gene panel for non-invasive diagnosis of biopsy-confirmed acute renal allograft rejection. Within the pediatric cohort of 200 samples, 177 samples were previously obtained as part of a prospective multicenter NIH/NIAID-funded clinical trial in which patients both with and without histological-graded AR were enrolled from 12 U.S. transplant centers (SNS01; NCT00141037; www.ClinicalTrials.gov; Li, L., et al. Am. J. Transplant. 2012, 12, 2710-2718). The remaining 23 samples were exclusively obtained for this study from the Laboratorio de Investigacion en Nefrologia, Hospital Infantil de Mexico. Within the adult cohort of 315 samples, samples were from obtained from 6 transplant centers in the U.S. and Europe (n=48: Emory University, Atlanta, Ga., Dept. of Surgery (Emory); n=97: University of California Los Angeles, Los Angeles, Calif., Immunogenetic Center (UCLA); n=92: University of Pittsburgh, Pittsburgh, Pa., E. Starzl Transplantation Center (Pittsburgh); n=39: California Pacific Medical Center, San Francisco, Calif. (CPMC); n=23: University of California San Francisco, San Francisco, Calif., Dept. of Nephrology (UCSF); n=16: Bellvitage University Hospital, Renal Transplant Unit Barcelona, Spain, (Barcelona)). The study was approved by all local IRB committees, and all patients agreed to participate by informed consent.

Each peripheral blood sample in this study was paired with a contemporaneous (within 48 hours) renal allograft biopsy from the same patient. Surveillance biopsies were obtained from all patients at engraftment, at 3, 6, 12, and 24 months post-transplantation, and at the times of suspected graft dysfunction. Multiple peripheral blood-biopsy pairs from the same patient were utilized as long as each biopsy had a conclusive phenotypic diagnosis. Each biopsy was scored by the center pathologist for each enrolling clinical site according to the Banff classification (Solez, K. et al. Am. J. Transplant., 2008, 8, 753-760; Mengel, M. et al. Am. J. Transplant. 2012, 12, 563-570). The peripheral blood-biopsy pairs were categorized as “acute rejection” (AR; n=130), “stable” (no-AR) or “other” diagnosis (Other). “Acute rejection” was defined for samples with a Banff tubulitis score (t) of ≥1 and an interstitial infiltrate score of ≥0. “Stable” was defined for samples displaying an absence of AR or any other substantial pathology. “Other” was defined for samples displaying an absence of Banff-graded AR, but either met the Banff criteria for chronic allograft injury (CAI; samples had IFTA grade ≥1; n=51), chronic calcineurin inhibitor toxicity (CNIT; n=19), BK viral infection (BKV; n=3), or other graft injury (OGI; n=153).

### Example 3: Patients

### Adult and Pediatric Set I

Table 5 shows the Adult and Pediatric Set I.

In one example, the combined pediatric and adult samples were separated into two groups for testing (n=236; 143 adult, 93 pediatric) and validation (n=292; 208 adult, 84 pediatric, Table 5).

### Adult and Pediatric Set II

In another example, the combined pediatric and adult samples were separated into three groups for training and testing (n=143 adult), for validation (n=124; 59 adult, 65 pediatric), and for independent prediction (n=191; 130 adult, 61 pediatric, Table 4).

Adult and Pediatric Set III

In another example, the combined pediatric and adult samples were separated into 100 samples for validation (77 adult, 23 pediatric, Table 4).

### Example 4: Blood Sample Collection and RNA Processing

### Blood Sample Collection

Blood was collected in 2.5 mL PAXgene™ Blood RNA Tubes (PreAnalytiX, Qiagen, Valencia, Calif.) or in Ficoll tubes for peripheral blood lymphocytes (PBL) isolation. PBL samples were only used for microarray discovery on Affymetrix systems. Total RNA was extracted using the column-based method kits of PreAnalytix (Qiagen) for PAXgene™ tubes or RNeasy (Qiagen) for PBL samples according to the manufacturer's protocol.

### RNA Extraction

Total RNA was measured for RNA integrity using the RNA 6000 Nano LabChip Kit on a 2100 Bioanalyzer (both from Agilent Technologies, Santa Clara, Calif.) with suitable RNA defined by an RNA integrity number (RIN) exceeding 7 (Fleige, S. and Pfaffl, M. W. Mol. Aspects. Med. 2006, 27, 126-139.; Schroeder, A. et al. BMC Mol. Biol. 2006, 7, 3).

cDNA Synthesis

cDNA synthesis was performed using 250 ng of extracted quality mRNA from the peripheral blood samples using the SuperScript® II first strand cDNA synthesis kit (Invitrogen, Carlsbad, Calif.) according to the manufacturer's protocol.

### Example 5: QPCR

### Total RNA Sample Preparation for Microfluidic QPCR

Samples were prepared for microfluidic qPCR analysis using 1.52 ng (relative amount) of total RNA from the cDNA synthesis for specific target amplification and sample dilution using pooled individual ABI Taqman assays for each gene investigated, excluding 18S, according to Fluidigm's protocol (Fluidigm, South San Francisco, Calif.). Briefly, specific target amplification was performed using 1.52 ng of cDNA in the pooled Taqman assays in multiplex with Taqman PreAmp Master mix (ABI) in a final volume of 5 μL. Amplification was achieved following 18 cycles in a thermal cycler (Eppendorf Vapo-Protect, Hamburg, Germany). Samples were subsequently diluted 1:5 with sterile water (Gibco, Invitrogen, Carlsbad, Calif.).

### QPCR

Microfluidic qPCR was performed on the 96.96 Dynamic Array (Fluidigm) using 2.25 μL of the diluted sample obtained from the specific target amplification, along with Taqman Assays (Applied Biosystems, Foster City, Calif.) for each mRNA, Taqman Universal master mix (Applied Biosystems), and loading reagent (Fluidigm) as outlined in the manufacturer's protocol. The chip was primed and loaded via the HX IFC Controller (Fluidigm) and qPCR was performed in the BioMark (Fluidgm) using default parameters for gene expression data collection, as indicated in the manufacturer's protocol (Fluidigm). Standard Comparative Ct values were used to determine the relative fold change values of gene expression using 18S as the internal endogenous control reference and Universal Human Total RNA as the external comparative reference (Qiagen, Venlo, Limburg).

### ABI QPCR

Standard protocols were followed for qPCR reactions on the ABI 7900 Sequence Detection System or the ViiA7 (Applied Biosystems) using standard conditions (10 min at 95° C., 40 cycles of 15 sec. at 95° C., 30 sec. at 60° C.) and gene expression assays (Applied Biosystems). The relative amount of RNA expression was calculated using a comparative Ct method. Expression values were normalized to 18S using a ribosomal RNA endogenous reference and a Universal human Total RNA (Qiagen.).

### Example 6: Data Pre-Processing and Normalization

### Microfluidic QPCR Data/Pre-Processing and Normalization

Raw Ct values from 42,792 qPCR reactions performed on RNA from 236 adult and pediatric samples to measure the expression of a larger gene panel of 43 genes using the Fluidigm high-throughput microfluidic qPCR technology were collected from six 96.96 microfluidic chips (Fluidigm). Ct values were extracted by Fluidigm Real-Time PCR analysis software and uploaded into Excel (Microsoft Office 2012, Microsoft, CA). Mean Ct values for technical replicates were calculated if standard deviations were <0.5 for the replicates. Ct values were normalized against an endogenous control gene using the delta Ct (dCt) method (Livak, K. J. and Schmittgen, T. D. Methods 2001, 25, 402-408). Four different control genes, ribosomal 18S, beta actin (ACTB), glyceraldehyde phosphate dehydrogenases (GAPDH), and beta-2 microglobulin (B2M), were tested. Because 18S showed the least variability across all samples, it was selected for calculation of dCt values. Missing values were inputed by K nearest neighbor (KNN; Troyanskaya, O. et al. Bioinformatics 2001, 17, 520-525) with 5 neighbors. Visualization of the raw qPCR data was achieved in Partek Genomics Suite v. 6.6 (Partek Inc., USA) using unsupervised Principal Component Analysis (PCA) and hierarchical clustering.

Confounding factors on gene expression were identified by PCA and Analysis of Variance (ANOVA), and were corrected by Batch Effect Removal in Partek (mixed model ANOVA combining categorical and numerical factors) and by using the empirical Bayes method with the combat function in the SVAR package. This method is robust for outliers in small samples (Chapelle, O., Haffner, P., and Vapnik, V. N. IEEE Trans. Neural Netw. 1999, 10, 1055-1064). Normalized expression data for the larger panel of 43 genes was subsequently used for identification of differentially expressed genes between AR and no-AR, for better understanding of the mechanisms of AR across different age groups, and for the selection of genes with highest predictive power, sensitivity, and specificity for AR, as outlined below.

### Correcting for Confounders in Microfluidic QPCR Data Using Batch Effect Removal in Partek

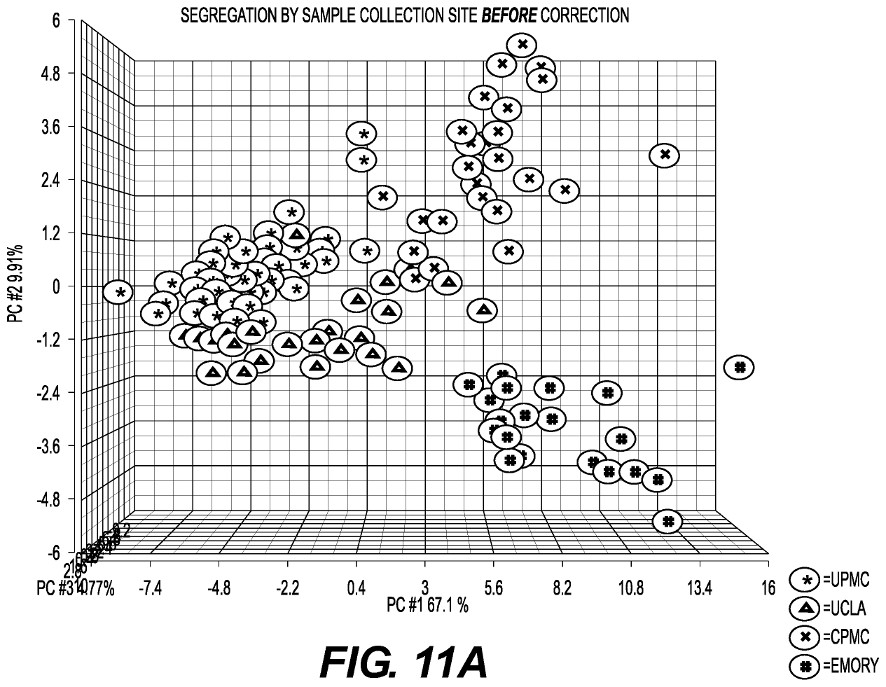

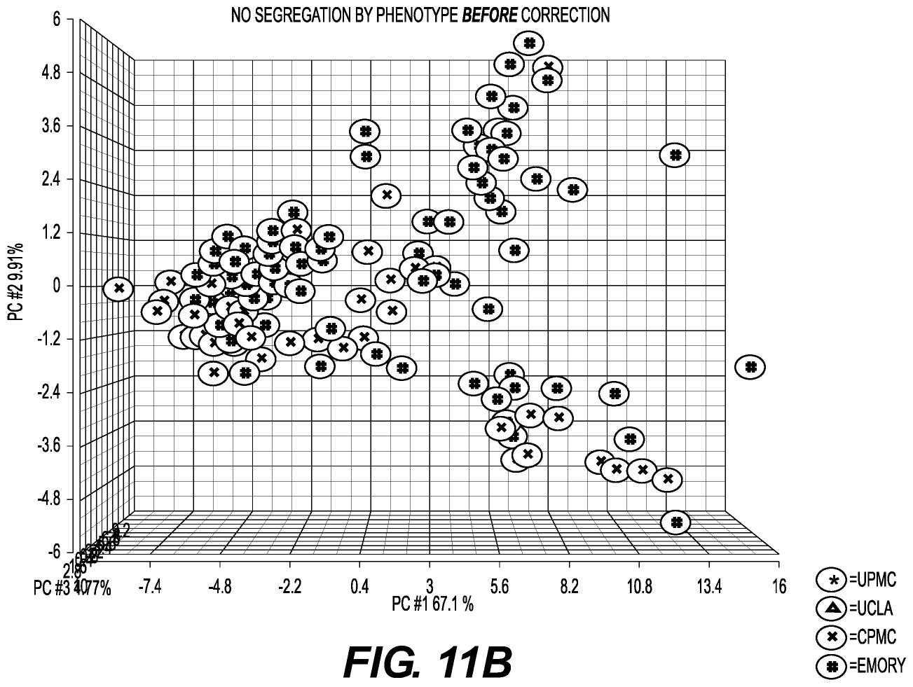

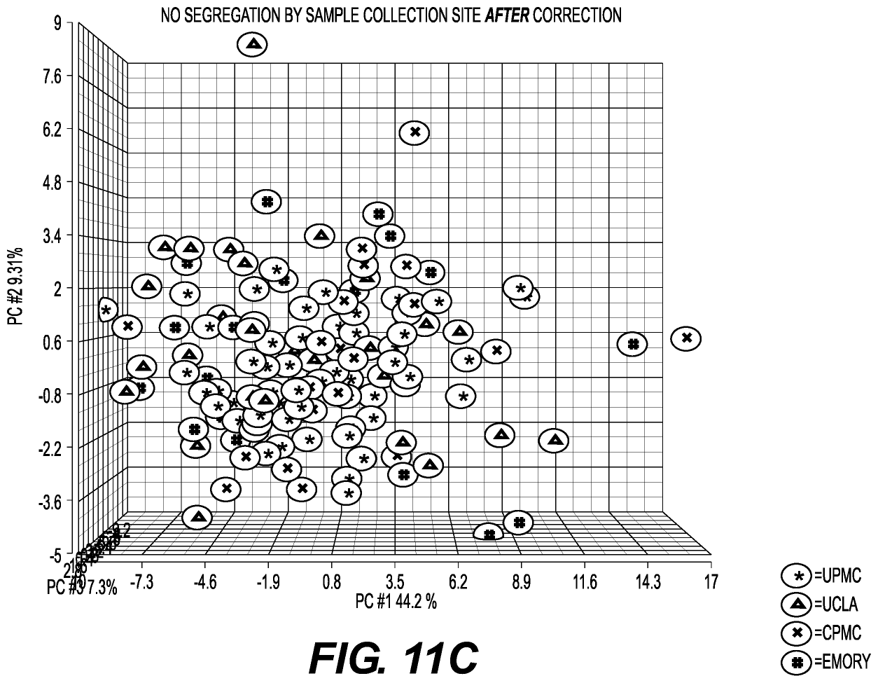

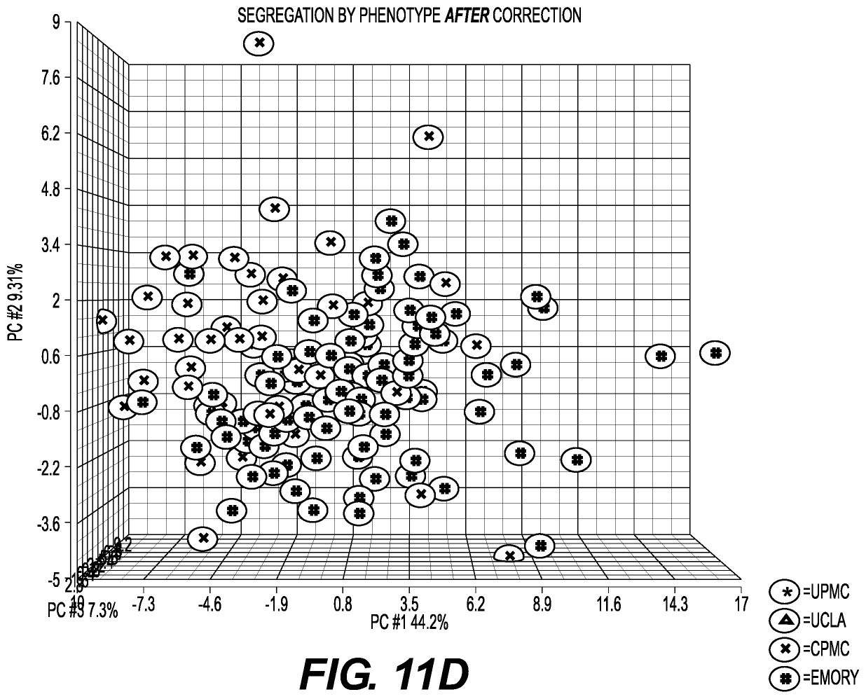

In the adult dataset of 143 AR and no-AR samples, the technical effects of RNA source, PCR plate, and the external effect of transplant center on differential gene expression across the samples was evaluated in a mixed ANOVA model. RNA source, PCR plate, and transplant center were included as random categorical factors, and phenotype (AR, no-AR) was included as a categorical factor. P-values were calculated for each factor and a p-value of <0.05 indicated that the differential expression of a particular gene related to either one of the factors included in the ANOVA. The batch effect removal feature in Partek, based on an ANOVA model, was initially designed to remove the effects of differential gene expression in microarray data when microarray chips were hybridized in different batches. Subsequently, this feature was utilized to correct for unwanted random factors of RNA source, PCR plate, and transplant center by building a mixed 4-way ANOVA model that adjusted the data so that p-values for RNA source, PCR plate, and transplant center became 1. In this way, no differences in gene expression due to these factors were present and the p-values for phenotype were maximized (FIGS. 11A-D).

Principal component analysis (PCA) of QPCR data from 143 AR and No-AR adult samples (Cohort 1) for 43 rejection genes revealed sample segregation by sample collection site (FIG. 11A) rather than phenotype (FIG. 11B). Normalization of QPCR data by mixed ANOVA corrected for the dominant effect of sample collection site on gene expression (FIG. 11C) and resulted in segregation of samples into AR and No-AR (FIG. 11D). PCA was performed using relative gene expression values (dCt 18S) for 43 genes. A mixed ANOVA model was built with sample collection site, RNA source and chip as random categorical factors and phenotype as categorical factor. Each sphere represents a sample; symbols reflect sample collection sites (*=UPMC; Δ=UCLA; X=CPMC; #=EMORY); the figure also reflects patient phenotype (AR; No-AR) based on biopsy diagnosis.

### Correcting for Confounders in Microfluidic QPCR Data Using Empirical Bayes Method in R

Prior to variable selection in the adult data set of 143 AR and NO-AR samples, the expression of the 43 genes was normalized using the empirical Bayes method with the combat function in the SVA R package to remove batch effect. This method is robust for outliers in small samples.

### Processing and Normalization of Abi QPCR Data

Raw Ct values from ABI QPCR reactions performed on RNA from 100 adult and pediatric samples to measure the expression of 17 genes were collected from 384 well plates. Ct values were extracted by ABI viia7 PCR analysis software and uploaded into Excel (Microsoft Office 2012, Microsoft, CA). Mean Ct values for technical replicates were calculated if standard deviations were <0.5 for the replicates. Ct values were normalized against ribosomal 18S RNA as endogenous control gene for calculation of delta Ct values (dCt) and additional against human Universal RNA (Qiagen) for calculation of deltadelta Ct values (ddCt) using the method described here (Livak, K. J. and Schmittgen, T. D. Methods 2001, 25, 402-408).

### Example 7: Methods for Selection AR and No-AR Specific Genes

A total of 43 genes were used for selection of AR and No-AR specific genes. Genes were identified to be differentially altered and associated with AR compared to stable allografts (Table 2) based on previous microarray studies in pediatric and in adult transplant rejection (Li, L. et al. Am. J. Transplant. 2012, 12, 2710-2718; Naesens, M. et al. Kidney Int. 2011, 80, 1364-1376; Sarwal, M. et al. N. Engl. J. Med. 2003, 349, 125-138). Of the 43 total genes, 10 (CFLAR, DUSP1, IFNGR1, ITGAX, MAPK9, NAMPT, NKTR, PSEN1, RNF130 and RYBP) were identified in previous work that focused on the development of a prediction model of AR in pediatric renal transplantation (Li, L. et al. Am. J Transplant. 2012, 12, 2710-2718). The remaining 33 genes were differentially altered, as determined by meta-analysis of microarray data, in AR as compared to stable allografts across various types of solid organ transplantation (Khatri et al. JEM, 2013, accepted for publication).

### Example 8: Methods for Identification of Differentially Expressed Genes Between AR and No-AR

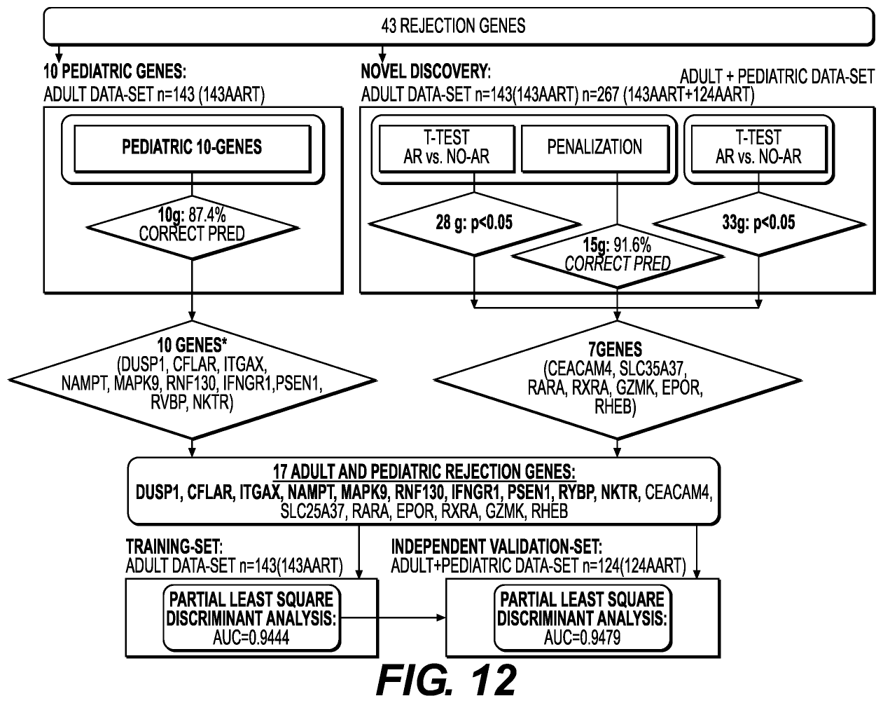

One- and multiple-way ANOVA, unpaired Student t-test with Welch correction in case of significantly different variances, and calculation of false discovery rate (FDR) to correct for multiple comparisons were used to detect significantly differentially expressed genes between AR and No-AR and to help understand the mechanisms of AR across age groups; a p-value of <0.05 or FDR <5% were considered significant (FIG. 12).

### Example 9: Methods for Identification of Genes Discriminating AR and No-AR

### Evaluation of Previously Published Genes in 143 Adult Samples

Previously published 10 gene panel (CFLAR, DUSP1, IFNGR1, ITGAX, MAPK9, NAMPT, NKTR, PSEN1, RNF130 and RYBP; Li, L. et al., Am. J Transplant 2012, 12, 2710-2718) from the larger panel of 43 genes was used for discrimination and prediction of the AR phenotype in the adult test dataset of 143 samples (FIG. 12)

### Identification of Novel Genes in 236 Adult and Pediatric Samples

To define a novel gene panel independent of age, transplant center, and RNA source and with high predictive power for AR using a minimal number of genes, Shrinking Centroids (Tibshirani, R. et al. Proc. Natl. Acad. Sci. USA 2002, 99, 6567-6572; Storey, J. D. and Tibshirani, R. Proc. Natl. Acad. Sci. USA 2003, 100, 9440-9445), forward and backward selection, and genetic algorithm (Zhu, Z. et al., IEEE Trans. Syst. Man. Cybern. B Cybern. 2007, 37, 70-76) were applied in the combined adult and pediatric dataset of 236 samples. In addition, an exhaustive search was applied to define top-ranked genes by searching through all possible combinations of 5 genes from the 43 genes analyzed in the 236 samples. In the Shrinking Centroids approach, all possible gene combinations from the 43 genes were tested with increments of 1 gene at a time, with the minimum number of genes set at 5 and the maximum number of genes set at 20. Genes were tested for their predictive probability of AR by cross validation (1-LLOCV). A total of 1872 models were tested using 117 different algorithms. Results were ranked according to the number of genes, and the AUC for the same gene combinations ranging from 5 genes to 43 genes were averaged. The resulting combination with the highest average AUC was selected. In the forward selection (step up) and genetic algorithm, incremental numbers of gene panels were tested: n=5, 7, 10, 12, 13, 15, 17, and 20 were each tested with 117 different algorithms as described above. Results were compared and final genes, selected in at least 50% of the models, were chosen. In the genetic algorithm, the initial population from which a gene panel was randomly selected was defined such that each gene was tested at least 50 times. The following populations were tested: 430, 308, 215, 180, 166, 127, and 108 for gene panels of n=5, 7, 10, 12, 13, 17, and 20, respectively, according to the equation:

$$N = \frac{43 * 50}{n}$$

where N is the initial population size, n is the size of the gene panel, 50 represents the times a gene has to appear in the initial population, and 43 is the total number of genes to be drawn from (FIG. 12)

### Identification of Novel Genes in 143 Adult Samples

Prior to variable selection, the genes were normalized by using the empirical Bayes method with the combat function in the SVA R package to remove batch effect. This method is robust for outliers in small samples. Since the data is sparse with 143 observations and 43 genes, we used penalized logistic regression to classify patient samples using the glmnet R package. This approach provides not only accurate estimates for the regression coefficients but also probability estimates for each patient. We used the regularization paths for generalized linear models via Coordinate Descent for the estimations (FIG. 12).

### Example 10: Methods for Evaluation of Genes for Discriminating AR and No-AR

Gene selections were evaluated for discrimination and prediction of AR by Discriminant Analyses (DA) with equal and proportional prior probability, Support Vector Machine (SVM), logistic regression (LR) and partial least square DA with equal prior probability (Chapelle, O. et al., IEEE Trans. Neural Netw. 1999, 10, 1055-1064; Brown, M. P. et al., Proc. Nat. Acad. Sci. 2000, 97, 262-267) with kernel function radial basis function (rbf), partial least square (pls) DA (Perez-Enciso, M. and Tenenhaus, M. Hum. Genet. 2003, 112, 581-592; Gottfries, J. et al., Dementia 1995, 6, 83-88). SVM classification uses the regularization paths radial basis function (rbf) to find the best generalized non-linear vectors (“support vectors”) that would define decision planes which provided the widest separation of AR and no-AR by simultaneously minimizing the empirical classification error and maximizing the geometric margin. SVM performs well on data-sets with sparse numbers of features (genes) and samples (Nouretdinov, I. et al., Neuroimage 2011, 56, 809-813). To minimize type 1-error, a ten-fold one level leave one-out cross validation (1-LLOCV) was performed rather than dividing the dataset into separate training and test sets. Area under the curve (AUC) and posterior probability for AR was given for each classification method to assess the predictive power, sensitivity, and specificity for AR by these genes in the combined adult and pediatric dataset. Genes with the highest predictive power for AR, the highest sensitivity, and the highest specificity from each gene selection approach were compared for a final selection of 17 genes for qPCR on the ABI platform (Abi viia7, Life Technologies, Foster City, Calif.). P-values and FDR values from Student T-test and ANOVA comparing AR and no-AR were used when needed. The workflow for final gene selection is shown in FIG. 12.

### Example 11: Methods for Development of an Algorithm for Classification of AR and No-AR in Fluidigm QPCR Data

### Identification of an Algorithm in 236 Pediatric and Adult Samples Using 17 Genes

A total of 122 classification algorithms were tested using the selected genes (17 in total) with two level-leave one out nested cross validation (2-LLOCV) and 5 outer and 5 inner data partitions. The “inner” cross validation (CV) was performed in order to select predictor variables and optimal model parameters, and the “outer” CV was used to produce overall accuracy estimates for the classifier. “Inner” CV was performed on the training data not held out as test data by the outer CV in order to select the optimal model to be applied to the held out test set. Classification models tested in the 236 samples included discriminant functions and equal or proportional prior probabilities, KNN with Euclidean, average Euclidean or cosine dissimilarity distance measures and 5 neighbors, nearest centroids with equal or proportional prior probabilities, LR, and SVM.

### Identification of an Algorithm in 143 Adult Samples Using 17 Genes

A total of 122 classification algorithms were tested using the selected genes (17 in total) with two level-leave one out nested cross validation (2-LLOCV) and 10 outer and 10 inner data partitions in 143 adult samples. The “inner” cross validation (CV) was performed in order to select predictor variables and optimal model parameters, and the “outer” CV was used to produce overall accuracy estimates for the classifier. “Inner” CV was performed on the training data not held out as test data by the outer CV in order to select the optimal model to be applied to the held out test set. Classification models tested in the 143 samples included partial least square- and linear-Discriminant analysis with equal and proportional prior probability, support vector machine, KNN with Euclidean, average Euclidean or cosine dissimilarity distance measures and 5 neighbors, nearest centroid with equal or proportional prior probabilities, and LR. Top models were evaluated in 143 samples with 1-LLOCV. Measures of accuracy were correct rate, sensitivity, specificity, NPV, PPV, and the area under the receiver operator curve (AUC).

### Identification of an Algorithm in 143 Adult Samples Using 15 Genes

We fitted 100 Elastic Net logistic regression models to the 43 genes using bootstrapped samples (29 test, 114 training, sampled with replacement) to classify AR vs. No-AR. For each bootstrap a nested cross-validation loop estimated the best value for X, according to the deviance. The a parameter of the Elastic-Net was fixed at 0.95, the value recommended by. In order to rank the genes we counted the number of times each gene was selected by the Elastic-Net over the 100 bootstraps. For each of the bootstrap samples, the Elastic-Net fits a subset of the 43 genes with non-zero coefficients. After running the 100 bootstrapped models, we selected K genes with the greatest number of non-zero coefficients. In a second step, in order to have a unbiased estimation of the predictive performance (classification rate, sensitivity, specificity, PPv, NPv), we ran another set of 100 bootstrap Elastic-Net classifications with nested cross-validation for X, this time using only the set of K genes selected in step 1. We report classification rates, sensitivity, specificity, positive predictive values (PPv) and negative predictive values (NPv).

### Example 12: Methods for Development of an Algorithm for Discrimination and Prediction of AR and No-AR in Fluidigm and ABI QPCR Data

### Development of a Correlation Based AR and No-AR Classification

To calculate a Pearson's correlation coefficient (ρ) for each patient sample, delta-Ct values were used for a queried sample compared to the mean gene delta-Ct values for either AR or no-AR classified samples.

To calculate a Pearson's correlation coefficient (ρ) for each patient sample, deltadelta-Ct values were used for a queried sample compared to the mean gene deltadelta-Ct values for either AR or no-AR classified samples.

Z-scores are calculated for each sample ρ, relative to the average (μ) and standard deviation (σ) of all ρ values from all sample comparison, as follows:

$$z = \frac{x - {μ}_{global}}{{σ}_{global}}$$

Samples were classified as AR or no-AR based on comparison of the sample AR and no-AR z-scores (greater z in AR or no-AR). These functions can be found in the LineageProfilerlterate.py module of AltAnalyze.

The correlation analysis was performed for all possible combinations of 4, 5, 6, 7, 8, 9, 10, 11 and 12 gene sets, where applicable. The best reported models for the ABI analyses were scored based on the percentage of correct classified patient samples out of the total, when comparing gene sets of different sizes.

### Development of LineageProfiler as a Correlation Based Algorithm for Classification of AR and No-AR

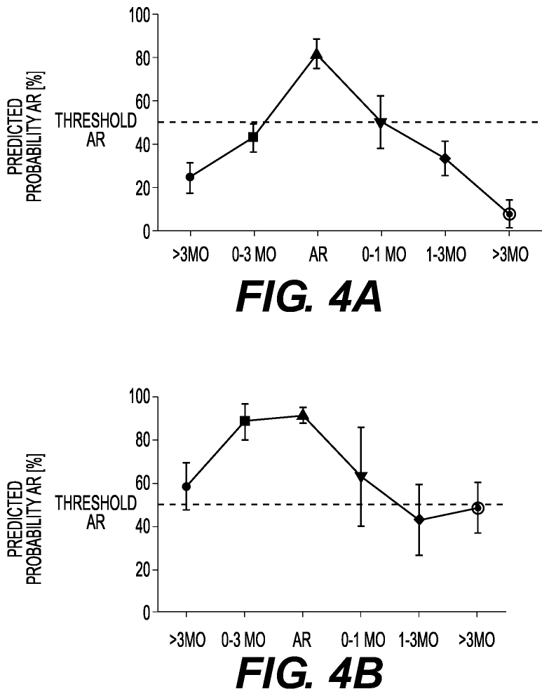

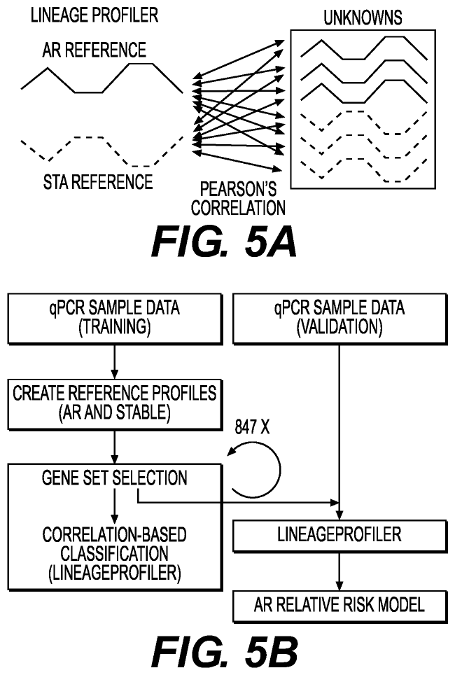

A new correlation-based, open-source algorithm named LineageProfiler (LP) was used and further modified for the discovery of an optimal gene model for further qPCR evaluation. The input for LP is delta delta-Ct normalized patient sample qPCR values and two reference qPCR profiles (an AR reference profile and a no-AR reference profile). This analysis consisted of 5 steps: Step 1: importing a matrix of RNA expression values for a panel of evaluated genes; Step 2: for each gene, creating and storing a single reference expression vector (mean) from all AR samples and a single reference expression vector for all no-AR samples; Step 3: identifying all possible combinations of genes analyzed for each qPCR set (gene sets); Step 4: directly comparing each patient RNA profile to the reference AR profile and the reference no-AR profile for each gene set in order to classify the patient sample (using LP); and Step 5: ranking gene sets based on known AR and no-AR status in order to identify the top prognostic lists for associated reference profiles. Gene sets from the 17 genes of several lengths, ranging from 4 to 12, were created for each distinct measurement platform (Fluidigm or ABI) and for all possible combinations. For the Fluidigm analysis, an optimization function was written that iteratively identifies the top-scoring model starting with all genes, and further analyzes all subsequent derivation of models. After the best performing gene sets were identified, these gene sets were fixed and applied to distinct validation datasets. Analysis of existing or new datasets with the corresponding reference expression profiles can be achieved in the open-source software AltAnalyze version 2.0.8 (http://www.altanalyze.org) using the LP function (FIGS. 4A-B)

Development of kSAS as a Correlation Based Algorithm for Classification of AR and No-AR

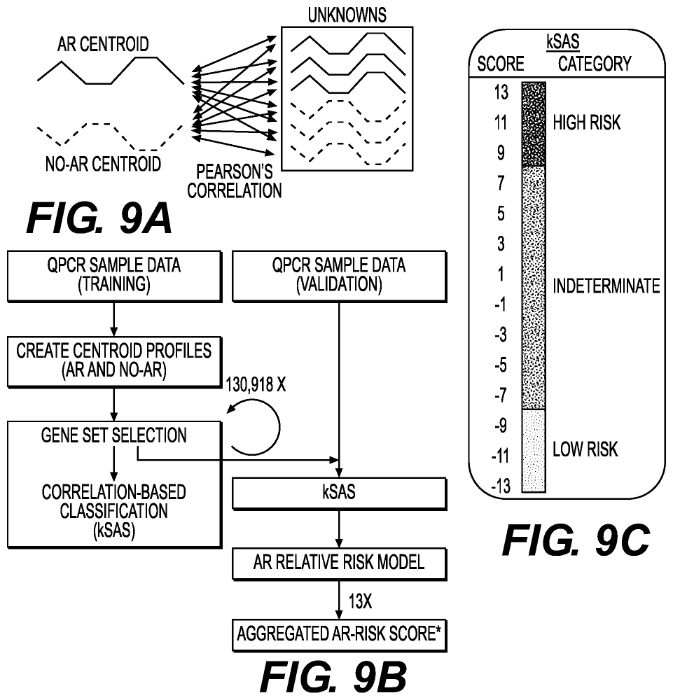

For robust risk stratification of samples as AR or No-AR, a new correlation-based algorithm named kSAS was developed. Rather than correcting external confounders by methods such as empirical Bayes method and ANOVA which are suitable approaches in discovery and cross-validation analyses where large data-sets are evaluated, kSAS was developed to apply fixed AR and No-AR QPCR reference profiles for the 17 gene-panel allowing accurate prospective prediction of samples independent of number, sample collection site and thus more applicable for routine clinical settings. kSAS uses QPCR dCt (18S) values in patient samples, and in two reference QPCR profiles (one for known AR and one for known No-AR). The kSAS analysis comprises 5 main steps for training and testing: 1) import the 17 gene dCt(18S) expression matrix for all samples, 2) define known AR and No-AR expression vectors for each gene; 3) identify all possible combinations of genes using an optimization function which identifies the top-scoring model iteratively starting with all genes 4) compare all resulting models for each patient to the reference AR and No-AR profile to classify the patient sample based on the degree of correlation (Pearson Correlation Coefficient); 5) rank gene sets by correlation to identify the top prognostic models. To calculate a Pearson's correlation coefficient (p) for each patient sample we compared dCt(18S) values of each gene in a queried sample to the mean dCt(18S) value of the same gene in either the AR or No-AR reference. For each resulting gene model a risk score was calculated by calculating the AR p minus No-AR p times 10. All resulting model risk scores were summed to provide an aggregated AR risk score for each sample. Samples were classified as AR or No-AR based on comparison of the sample AR and No-AR risk scores (greater correlation in AR or No-AR). The correlation analysis was performed for all possible combinations of 4, 5, 6, 7, 8, 9, 10, 11 and 12 gene sets, where applicable. The best reported models were scored based on the percentage of correctly classified patient samples out of the total, when comparing gene sets of different sizes. Exemplary gene sets are in Table 2. To address collection-site associated variances in AR and No-AR profiles, a separate AR and No-AR reference for each collection site was provided in a single table to select the most highly correlated site reference pair for each individual sample comparison when computing the correlation derived risk score for each model (FIG. 9A-C)

### Creating New Reference Data for Correlation Based Classifications of AR and No-AR

To use a reference for a new transplant center, blood classified as AR or no-AR, collected in the same manner as the unknown samples, should be collected and profiled using the recommended 12-gene model set (see below) prior to analysis of the unknown samples. These samples serve as transplant center-specific references, since machine and sample collection center bias have previously been observed. After obtaining qPCR profiles for a sufficient number of samples, the mean expression of all AR and no-AR samples is taken separately to create a two-column reference for all genes assayed. Alternatively, the use of a pooled RNA reference instead of individual samples should be sufficient. The data are saved as a three column tab-delimited text file, with the first column containing the gene IDs, and the second and third column containing the AR and no-AR references, respectively. Re-analysis of the original samples used for this reference is initially recommended to determine if significant variability among these reference samples exist (e.g., poor classification scores between AR and no-AR samples).

### Example 13: Methods for Evaluation of a Correlation Based Algorithm for Discrimination and Prediction of AR and No-AR in Fluidigm and ABI QPCR Data

Evaluation of kSAS in Non-Transplant Data

Prior to applying kSAS to AR and No-AR patient data, we evaluated this approach upon a previously described QPCR analysis of 50 breast cancer prognostic marker genes applied to 814 samples from the GEICAM/9906 clinical trial). kSAS was able to successfully classify a randomly selected patient test set (272 patient samples) into five distinct prognostic breast cancer groups, following reference creation (training) on the remaining samples, with a >85% success rate using all 50 marker genes. Smaller prognostic gene models of 24 and 25 genes were also able to classify patients at a higher percentage in the training set (90.0% versus 85.6%) and equivalent accuracy in the test set (83.1-83.8%).

Evaluation of kSAS in 143 Adult Fluidigm QPCR Data

We evaluated kSAS in the same normalized dataset of 143 adult samples (Cohort 1). Reference AR and No-AR profiles were obtained for all 43 genes from a random ⅔rds training sample set from Cohort 1. This training set was then further subdivided programmatically into 10 AR/No-AR equal sized ⅔rd and ⅓rd sets to identify top-scoring gene models. The highest scoring model from this training set was evaluated on the original ⅓rd training set using training set AR and No-AR reference profiles.

Evaluation of kSAS in 100 Adult and Pediatric ABI Viia QPCR Data

We evaluated the combined ability of all 13 12-gene models defined by kSAS to provide a single confidence score for each patient that is not based on a single gene model but includes all 13 12-gene models in 100 adult and pediatric samples. We calculated aggregated AR Risk-Scores for the combined data-set of 100 AR and No-AR samples (26 AR, 42 No-AR). The aggregated AR risk analysis produced a numerical AR Risk-Score for each patient (−13 to 13), by subtracting the times a patient was predicted as No-AR by the 13 12-gene models from the times the same patient was predicted as AR. Based on the aggregated risk-score patients can be categorized as High-Risk AR, as Low-Risk AR or as Indeterminate Risk. The cutoff for High Risk AR was an aggregated Score ≥9, for Low-Risk AR an aggregated Score ≤−9. Patients with aggregated scores ≥−7 and ≤7 were considered at indeterminate Risk (FIG. 9C)

### Example 14: Methods for Development of a Software for Correlation Based Algorithms for Classification of AR and No-AR

The correlation-based analyses described herein can be performed in AltAnalyze version 2.0.8 or later. LineageProfiler is available through a graphical user interface in the open-source software AltAnalyze (http://code.google.com/p/altanalyze/downloads, version 2.0.8 or higher) and as standalone python script (https://github.com/nsalomonis/LineageProfilerIterate). AltAnalyze can be downloaded from http://www.altanalyze.org, extracted to a hard drive, and installed with the latest human database when prompted (currently EnsMart65) following the initial launch. Alternatively, LineageProfiler functions can be performed using a command-line version of this software along with options for gene model discovery available at https://github.com/nsalomonis/LineageProfilerIterate. Instructions on running the standalone graphical user interface version of LineageProfiler and the command-line versions are described at http://code.google.com/p/altanalyze/wiki/SampleClassification. The source code for LineageProfiler was modified for use in the embodiments described herein, resulting in LineageProfiler Iterate. As used herein, LineageProfiler Iterate, modified LineageProfiler, and kSAS are used interchangeably. The source code for kSAS, is provided in Appendix C. This software can be used to classify quantitative expression values for a given set of samples as belonging to a particular disease class, phenotype, or treatment category. In brief, the algorithm does this by correlating an input set of expression values for a given sample to 2 or more reference conditions. Rather than correlating the sample with the references directly, a subset of genes can be selected from a model file, which has been previously identified to produce a high degree of predictive success using samples belonging to known classes. The algorithm can also be applied to new data to discover alternative or new gene models.

Development of Expression Files for AR and No-AR Classification in kSAS Using deltaCt Values (dCt)

AR classification is performed using qPCR derived expression values for a panel of AR- and No-AR discriminating genes, along with the control 18S gene. Delta-Ct values produced from qPCR on an ABI viia7 platform (relative to 18S) are used as the unknown sample input for this algorithm. In addition, a reference file containing a reference AR and reference no-AR profile (dCt) is also supplied to the software.

Development of Expression Files for AR and No-AR Classification in kSAS Using deltaCt Values(dCt)

AR classification is performed using QPCR derived expression values for a panel of AR- and No-AR discriminating genes, along with the control 18S gene. Deltadelta-Ct values relative to 18S and a universal human RNA produced from QPCR on an ABI viia7 platform are used as the unknown sample inputs for this algorithm. In addition, a reference file containing a reference AR and reference no-AR profile (ddCt) is derived from the QPCR data.

Generating Expression Files for AR Classification in kSAS Using deltaCt Values

The expression file consists of normalized expression values (qPCR delta Ct values) in a tab-delimited text file format with the file extension .txt. The first column in this file contains IDs that match first column of the reference file (gene symbols), the first row contains sample names, and the remaining data consists of normalized expression values (i.e., delta Ct values).

The reference file is an agglomeration of AR and no-AR qPCR delta Ct values in the same range of values as that found in the Expression File. All gene symbols in this file should match those present in the expression file. When running the software, a warning will be given if the values in the reference and expression files have low overall correlations (<90%). Ideally, the reported range of correlation coefficients should be 0.92-0.96 or greater. In the case where they are not, the experiment may need to be repeated or evaluated for additional quality control.

Generating Expression Files for AR Classification in kSAS Using deltadeltaCt Values

The expression file consists of normalized expression values (qPCR delta delta Ct values) in a tab-delimited text file format with the file extension .txt. The first column in this file contains IDs that match first column of the reference file (gene symbols), the first row contains sample names, and the remaining data consists of normalized expression values (i.e., delta deltaCt values).

The reference file is an agglomeration of AR and no-AR qPCR delta deltaCt values in the same range of values as that found in the Expression File. All gene symbols in this file should match those present in the expression file. When running the software, a warning will be given if the values in the reference and expression files have low overall correlations (<90%). Ideally, the reported range of correlation coefficients should be 0.92-0.96 or greater. In the case where they are not, the experiment may need to be repeated or evaluated for additional quality control.

Using kSAS for AR and No-AR Classification in kSAS Via a Graphical User Interface

This algorithm is also available in the open-source analysis package AltAnalyze, which does not require any dependency installation. AltAnalyze is a large transcriptome analysis toolkit which contains a number of distinct analysis functions. Because AltAnalyze requires installation of large databases and contains a large number of menus, use of the command-line version of the script may be advised.

To install the current version of AltAnalyze, the following five steps can be followed: 1) go to http://code.google.com/p/altanalyze/downloads; 2) locate the most recent appropriate version for the given operating system and follow the download links; 3) extract the .zip or .dmg file to a hard-drive and an accessible location; 4) open the AltAnalyze program folder and double-click on the executable AltAnalyze.exe (Windows) or equivalent; 5) proceed to download a small database (e.g., Zea mays) and de-select the option for “Download/update all gene-set analysis databases” (the gene annotations provided are not needed for sample classification).

The input file consists of the expression file for the unknown samples. The reference file consists of the expression file for the reference AR and No-AR samples

The model file consists of gene symbols that match those in both the reference and expression input files, but correspond to a subset of the gene set. The standard AR classification panel consists of thirteen 12-gene models. This file can be re-used for every analysis.

The output of kSAS is a tab-delimited text file with a score associated with all reference profiles. This result file was produced for the analysis of the training set samples.

Using kSAS for AR and No-AR Classification Via Command-Line Options

Once the LineageProfilerlterate/kSAS script has been downloaded, it should be moved to an easily accessible location. Next, a terminal window should be opened (also called command-prompt on a PC). Instructions for opening a terminal or command prompt window on a given operating system can easily be found online. Next, in the terminal window, directories to the folder containing the LineageProfilerlterate/kSAS script should be accessed.

Generate three files: an input file, a reference file, and a model file.

To analyze delta-Ct expression values, supply LineageProfilerlterate/kSAS with the locations of three files containing delta-Ct values for the input and reference files. The command --i is for the sample delta-Ct expression values. The command --r is for the reference expression file. The command --m is for the supplied thirteen 12-gene models. After entering this command, various printouts will be seen. The results will now be saved to the indicated results directory.

To analyze delta delta-Ct expression values, supply LineageProfilerlterate/kSAS with the locations of the three files containing deltadelta Ct values for the input and reference files. The command --i is for the sample delta delta-Ct expression values. The command --r is for the reference expression file. The command --m is for the supplied thirteen 12-gene models. After entering this command, various printouts will be seen. The results will now be saved to the indicated results directory.

Running kSAS within AltAnalyze

After installing AltAnalyze using the above procedure, an analysis of input data may be run. For this, the appropriate expression, reference, and model files are required.

To run kSAS using delta-Ct values, the following 6 steps can be followed: 1) open AltAnalyze and select “Begin Analysis”; 2) select “Continue” in the platform analysis menu; 3) select “Additional Analyses” and continue; 4) select “Lineage Analysis” and continue; 4) provide the expression file (dCt), reference file (dCt) and model file, and continue; 5) the progress of the classification analysis will be printed out; and 6) when complete, select continue, and the results folder will be present in the location of the expression file.

To run kSAS using deltadelta-Ct values, the following 6 steps can be followed: 1) open AltAnalyze and select “Begin Analysis”; 2) select “Continue” in the platform analysis menu; 3) select “Additional Analyses” and continue; 4) select “Lineage Analysis” and continue; 4) provide the expression file (ddCt), reference file (ddCt) and model file, and continue; 5) the progress of the classification analysis will be printed out; and 6) when complete, select continue, and the results folder will be present in the location of the expression file.

Interpretation of Results Generated in kSAS

Multiple fields will be present in the results file in the folder SampleClassification. The tab delimited text file can be opened in Excel. The data are presented as follows:

Column A: Samples—indicates the sample names
Column B: AR Predicted Hits—indicates the number of Models where AR is predicted
Column C: No-AR Predicted Hits—indicates the number of Models where no-AR is predicted
Column D: Composite Prognostic Score—combined score of columns B-C
Column E: Median Z-Score Difference—Median Z-Score from columns G-S.
Column F: Prognostic Risk—overall predicted risk assessment
Columns G-S: AR Predicted Hits—individual scores for each sample and model.

The Prognostic Risk (column F) designates samples as “High Risk AR”, “Indeterminate Risk AR” and “Low Risk AR.” “Low Risk AR” is considered to be most similar to individuals with a histology-proven stable graft, whereas “High Risk AR” is most similar to biopsy-proven AR grafts. Indeterminate Risk is assigned to any sample with any disagreements between the 13 models in the prognostic evaluation.

In 40 samples from UCSF, one sample, a biopsy-proven AR, had 8 gene set predictions as AR and 5 gene set predictions as no-AR out of the 13 total gene sets, each gene set composed of 12 genes. Therefore, this sample was considered as at indeterminate risk.

### Example 15: Differentially Expressed Genes Between Adult and Pediatric AR and No-AR

### Differentially Expressed Genes Between 236 Adult and Pediatric AR and No-AR Samples

In order to identify genes that distinguished both adult and pediatric AR from no-AR patients and presented robust biomarkers for non-invasive detection of AR, the simultaneous measurement of the expression of the above-defined 43 genes (42 genes plus the housekeeping gene ribosomal RNA 18S) across 236 blood samples from adult and pediatric patients on the microfluidic high-throughput qPCR platform Fluidigm (Biomark, Fluidigm Inc.) was performed. When evaluated by unsupervised PCA and ANOVA, the specific transplant center (“Center”) where the patient received the allograft was found to be the greatest variable to account for patient segregation over rejection status. By unsupervised PCA, samples segregated by transplant center were found to override the differences in gene expression inferred by phenotype (AR vs. no-AR) in the uncorrected dataset. Correction of the data using a mixed ANOVA model where transplant center, RNA source, and qPCR chip were included as random categorical factors to be removed and phenotype (AR, no-AR) as a categorical factor to remain, resulted in gene expression that did not segregate samples by transplant center but rather segregated samples by phenotype. Analysis of this normalized set demonstrated that a large subset of these genes were differentially expressed between AR and no-AR (Student T-test: n=32, p<0.05).

### Differentially Expressed Genes Between 267 Adult and Pediatric AR and No-AR Samples

A total of 31 genes were differentially expressed between 267 both adult and pediatric AR and No-AR (Cohort 1, n=143; Cohort 2, n=124; FDR<5%, ANOVA with Bonferroni post-test). Interestingly, 8/10 gene pediatric panel, were significantly different (p<0.05) in adult samples

### Example 16: Classification of AR and No-AR Samples Using 10 Genes

### Classification of Adult AR and No-AR Samples Using 10 Genes Via Support Vector Machine

To evaluate the potential validity of these gene sets for AR classification across distinct collection centers, gender, blood RNA sample source, and recipient age, two distinct classification approaches available in Partek and R were utilized. In Partek, the SVM algorithm (cost parameter c=701, kernel function=radial basis function exp(−gamma ∥x−y∥\hat{ }2) with gamma=3) and in R, the penalized logistic regression using the Elastic-Net, were applied for classification of samples into AR or no-AR. Both classification algorithms are binary classifiers, and SVM is designed to maximize the margin to separate two classes so the trained model generalizes well on unseen data without overfitting the data. However, SVM is a non-probabilistic classifier and does not provide individual prediction accuracy scores. Logistic regression provides predictive probability scores for each sample. These methods were applied to the Center-normalized data using the previously published 10-gene pediatric model (CFLAR, DUSP1, IFNGR1, ITGAX, MAPK9, NAMPT, NKTR, PSEN1, RNF130, and RYBP), which was previously validated for AR detection in pediatric and young adult blood samples (92% accuracy, 91% sensitivity, and 94% specificity to detect AR), in a test set of 143 adult AR (n=47) and no-AR (n=96) samples with a matched biopsy reading and clinical function. Using the above described SVM, the same 10 genes detected AR in adults with 87% accuracy, 70% sensitivity, and 96% specificity when applied to the adult dataset of 143 samples.

Detection of AR in Pediatric Samples Up to 6 Months Before and 1 Month after AR Biopsy Using 10 Genes Via Logistic Regression

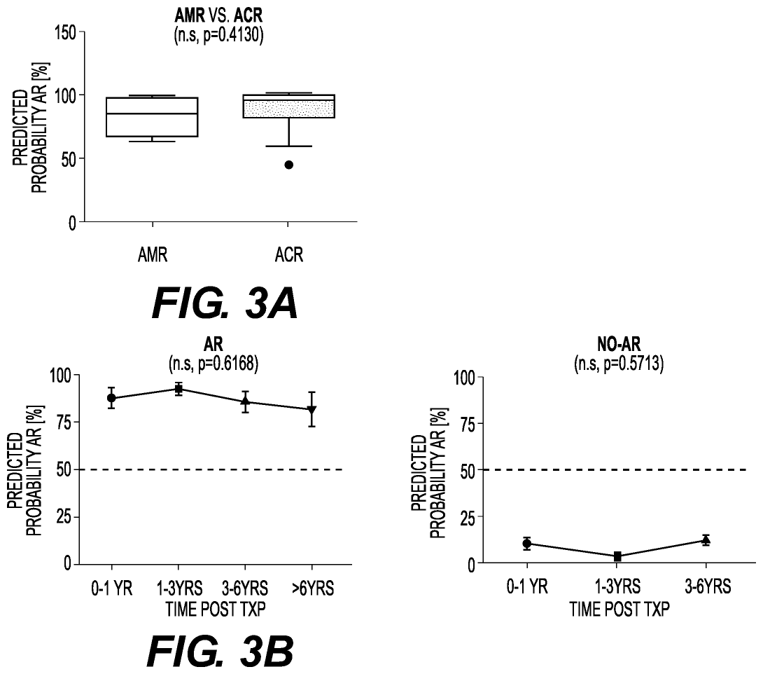

Serial samples from pediatric allograft recipients were available for 40 patients with biopsy proven AR collected up to 7 months before (n=27) and until 6 months after (n=30) the AR biopsy. The pediatric 5-gene expression model (DUSP1, NAMPT, PSEN1, MAPK9, and NKTR) revealed high AR prediction scores up to 6 months before (mean scores 0-3 mo=88%; mean scores 3-6 mo=58%) and until 1 month after (mean score=63%) the biopsy for AR. The mean score for 40 matched AR samples was 91%. In samples collected more than 1 month after the AR biopsy, mean predicted scores for AR were 42% after 3 months and 48% after >3 months (FIG. 3B).

### Example 17: Classification of AR and No-AR Samples Using 15 Genes

### Classification of 143 Adult AR and No-AR Samples Using 15 Genes Via Penalized Logistic Regression

In order to improve the accuracy and sensitivity, the influence of the additional 32 genes on the adult test set was examined using penalized logistic regression for the selection of additional genes that could be included to develop an age-independent AR prediction algorithm. As a result, 15 additional genes were selected from the 32 genes (CEACAM4, SLC25A37, RARA, CXCL10, GZB, IL2RB, RHEB, Clorf38, EPOR, GZMK, ABTB1, NFE2, FOXP3, MPP1, and MAP2K3). Use of these additional genes resulted in an improvement in the prediction of AR in the adult data set (92% accuracy, 86% sensitivity, and 94% specificity) via penalized logistic regression. Only 5 samples (2 no-AR and 3 AR) were incorrectly classified. The theta for AR prediction in this penalized logistic regression model was 50%, indicating that classification of the samples was achieved with a probability score of >50% for designating a sample as AR, and a probability score of <50% for designating the sample as no-AR (FIG. 1A)

### Classification of 49 Adult AR and No-AR Samples Using 15 Genes Via Penalized Logistic Regression

The performance of the adult 15-gene model in an independent set of 49 samples was tested. Samples included 8 AR and 6 no-AR patients having a biopsy-confirmed pathology report at the blood collection time. The remaining 20 samples were collected from patients who either did not have a matched biopsy at the blood sample collection time (N.A., n=22), or who were experiencing other forms of graft dysfunction (n=13) including acute tubular nephritis (ATN, n=3), acute drug toxicity (CNIT, n=4), or showed chronic allograft damage on biopsy (IF/TA, n=4), in addition to patients with BK nephropathy (BK, n=2). None of the 22 samples that originated from patients of unknown phenotype had a biopsy-proven rejection prior to or after sample collection. Using gene expression, all no-AR samples were correctly predicted as no-AR, and 5 of the 8 AR samples were correctly classified as AR. Prediction scores between AR and other sources of graft dysfunction were significantly higher in AR (p=0.0162). All samples from patients with unknown phenotype (N.A.) were predicted as no-AR (FIG. 1B).

### Equal Detection of Antibody-Mediated and Cellular Mediated Acute Rejection in Adults Using 15 Genes Via Penalized Logistic Regression

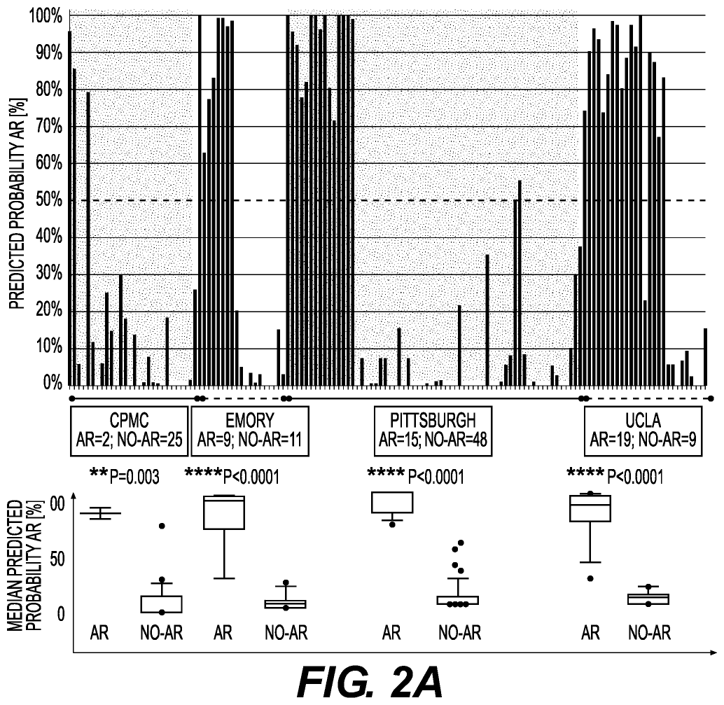

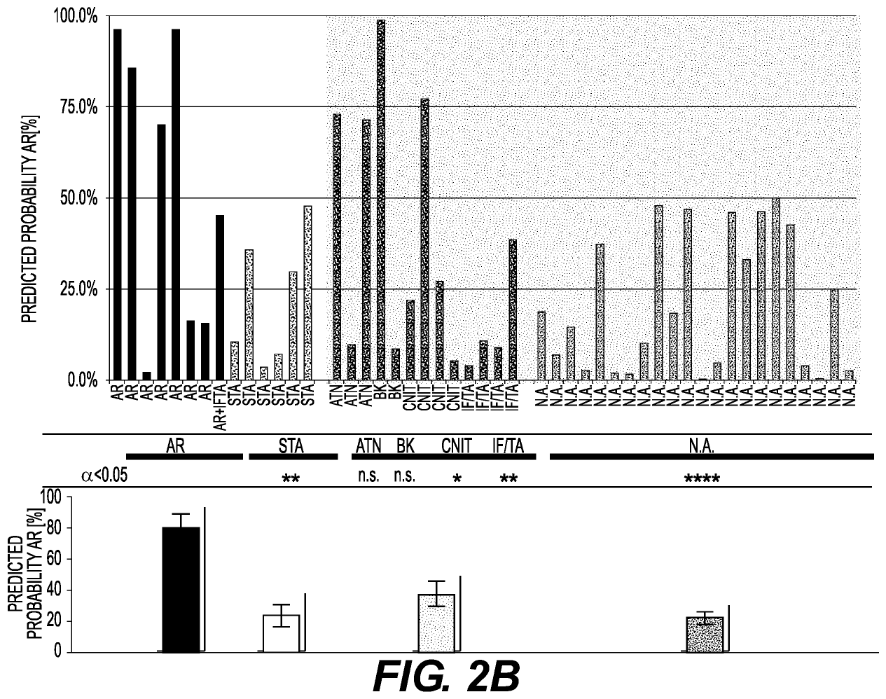

Most of the AR samples showed a mixture of cellular and humoral rejection. Donor specific antibody (DSA) data was not available at the time of biopsy in all cases. Using of a subset of 5 patients that showed only antibody-mediated rejection (AMR, C4D positive biopsy staining, DSA+), the prediction scores to patients having clean cellular mediated rejection (ACR, C4d−, DSA−; n=33) was compared. Although the number of pure antibody and cell-mediated rejection episodes is relatively small, comparison of the mean AR prediction scores in the 2 AR subgroups revealed that the model equally detected AMR and ACR with high prediction probability (mean Score AMR=82.9%±0.16; mean Score ACR=89.5%±0.12; p=0.413). (FIG. 2). FIG. 2A illustrates the predicted probabilities of AR in 143 AR and no-AR adult patients. FIG. 2B shows the predicted probabilities of AR in 49 independent patients (8 AR, 6 No-AR, 13 graft dysfunction, and 22 unknown).

Prediction of AR Prior 3 Months Prior to and 1 Month after AR Biopsy in Blood from Adult Renal Recipients Using 15 Genes Via Penalized Logistic Regression

Serial blood samples were available for a subset of patients with biopsy proven AR (n=59), collected up to 2 years before (n=23) and 1.5 years after (n=19) the AR biopsy. By gene expression, AR was indicated in the adult population up to 3 months before and until 1 month after the biopsy for AR (mean AR probability 0-3 months before=43%; mean AR probability 0-1 month after=50%). In blood samples collected more than 3 months before or 1 month after the AR biopsy, the probability of detecting AR using the gene expression model dropped to 24% and 24% probability, respectively. The mean score for the 17 matched AR samples was 82% (FIG. 3A).

### Example 18: Classification of AR and No-AR Using 17 Genes Via Support Vector Machine

In order to detect AR independent of recipient age, qPCR data from an independent subset of pediatric and young adult patients (Li, L. et al. Am. J. Transplant. 2012, 12, 2710-2718) consisting of 93 peripheral blood samples (22 AR, 71 no-AR) was combined with 143 samples from adult transplant recipients (47 AR, 96 no-AR). Using Shrinking Centroids, a set of 17 genes that classified patients into AR or no-AR were identified using the SVM algorithm with cost parameter=701, kernel=rbf, and gamma=3 for classification. This 17-gene model detected AR with 94% accuracy, 88% sensitivity, and 95% specificity in the combined dataset of 236 pediatric, young adult, and adult patients using SVM. This 17-gene set used a combination of 10 pediatric genes (CFLAR, DUSP1, ITGAX, RNF130, PSEN1, NKTR, RYBP, NAMPT, MAPK9, and IFNGR1), 6 of the newly defined 15 adult genes (CEACAM4, RHEB, GZMK, RARA, SLC25A37, and EPOR), as well as Retinoid X receptor alpha (RXRA). Using these 17 genes, only 8 of 69 AR samples were incorrectly predicted as no-AR, and only 8 of 169 no-AR samples were incorrectly predicted as AR. Clearly, the combination of adult specific and pediatric specific genes is necessary for the development of an age-independent prediction of AR with high accuracy, sensitivity, and specificity.

### Example 19: Classification of AR and No-AR Using 17 Genes Via Partial Least Square Discriminant Analysis with Equal Prior Probabilities

Classification of 143 Adult AR and No-AR Samples Using 17 Genes Via Partial Least Square Discriminant Analysis with Equal Prior Probabilities

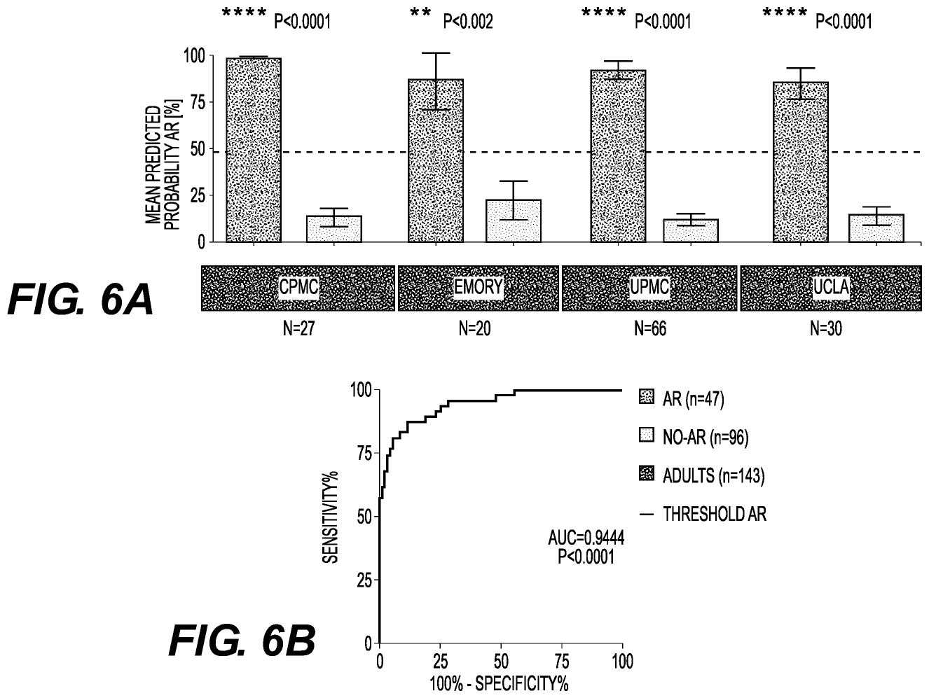

The final 17 genes to define the kidney AR prediction assay consisted of the pediatric 10 gene-panel (DUSP1, CFLAR, ITGAX, NAMPT, MAPK9, RNF130, IFNGR1, PSEN1, RYBP, NKTR) and additional 7 genes informative for adult rejection (SLC25A37, CEACAM4, RARA, RXRA, EPOR, GZMK, RHEB) (FIG. 12); these 17 genes showed optimized performance to discriminate AR across recipient ages: In the training set of 143 adult samples (Cohort 1) the 17 genes predicted 39/47 samples correctly as AR and 87/96 samples correctly as No-AR resulting in a sensitivity of 83%, and specificity of 91% in a partial least square Discriminant analysis with equal prior probability (plsDA; FIG. 6A-B). Mean predicted AR probabilities were highly significantly different comparing AR vs. No-AR in each center (CPMC: p<0.0001; Emory: p=0.002; UPMC: p<0.0001; UCLA: p<00001) (FIG. 6A). The overall area under the receiver operating characteristic (ROC) curve for the 17 genes was AUC=0.94 (95% CI 0.91-0.98; p<0.0001) by plsDA (FIG. 6B).

Classification of Independent 124 Adult and Pediatric Recipients Using 17 Genes Via Partial Least Square Discriminant Analysis with Equal Prior Probabilities

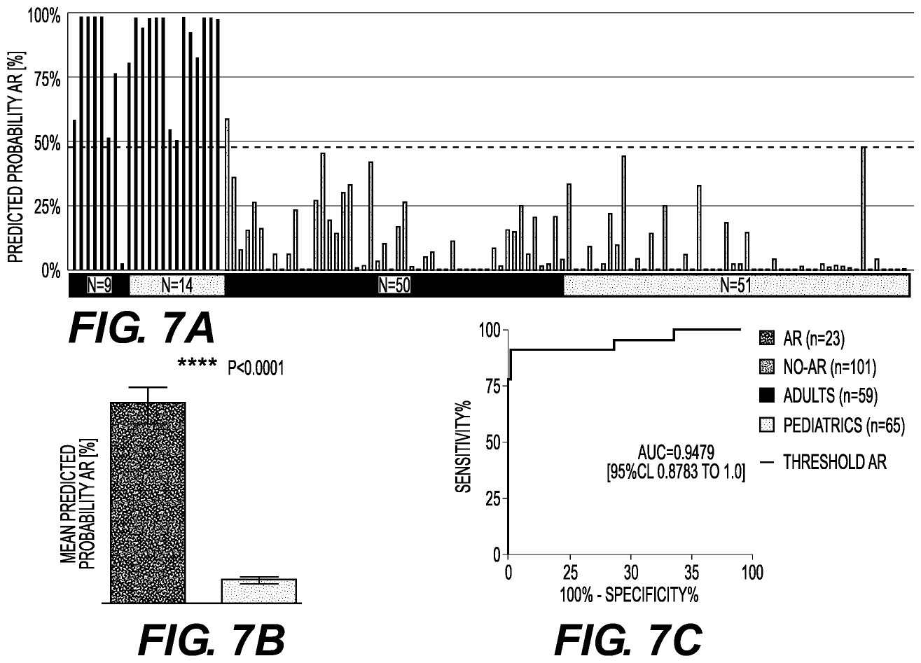

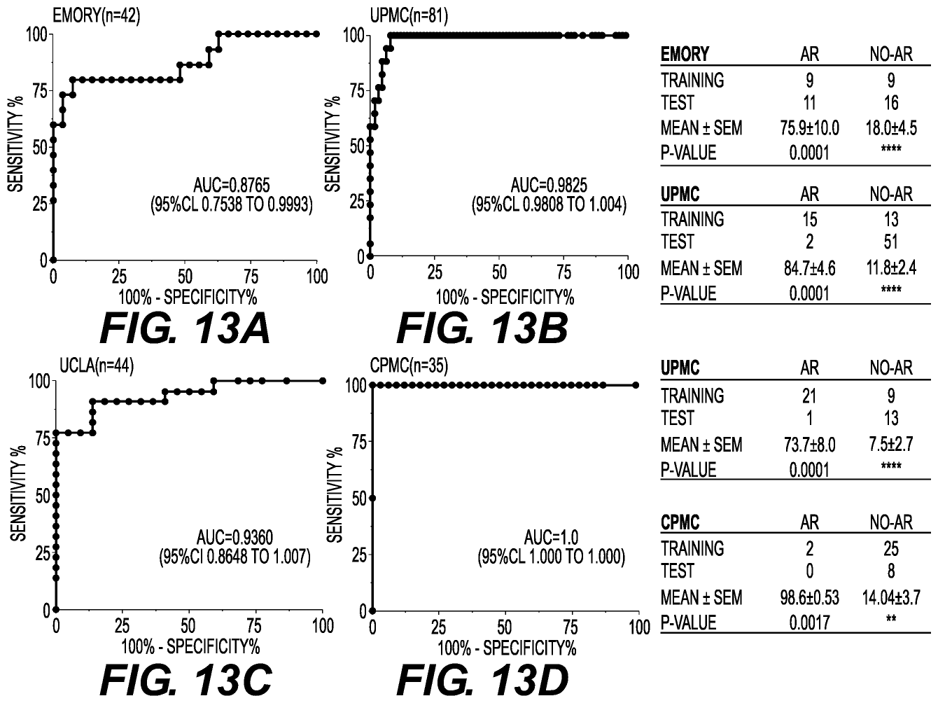

To independently validate the 17 gene kidney AR prediction assay model to discriminate AR from No-AR phenotypes in both adult and pediatric recipients, we tested its performance in a combined adult (n=59) and pediatric (n=65) set of 124 independent samples (Cohort 2; retrospective validation) also run on the Fluidigm platform. The 17 gene kidney AR prediction assay model predicted 21/23 samples correctly as AR and 100/101 samples correctly as No-AR (FIG. 7A), inclusive of 4 patients with BK viral nephritis, yielding an assay sensitivity of 91.3% and specificity of 99.01%. One of the 2 misclassified AR samples had severe chronic damage (IF/TA grade III) with >33% global obsolescence in the biopsy sample at time of rejection. As seen in the training-set (Cohort 1), mean predicted probabilities of AR were also significantly different between the AR (80.55%) and No-AR (9.2%) samples (p<0.0001; FIG. 7B) in the validation set (Cohort 2); mean predicted probabilities of AR in the BKV group was low at 12.76%. ROC analyses in the 124 samples resulted in an AUC=0.9479 (95% CI 0.88-1.0) (FIG. 7C). To evaluate the performance of the 17 gene kidney AR prediction assay model in each Sample Collection Site, we calculated ROC AUCs for predictions in Emory (n=42), UPMC (n=81), UCLA (n=44) and CPMC (n=35) from Cohort 1 and Cohort 2. The performance of the assay by transplant center showed individual ROC AUCs >0.8 for all 4 centers (FIG. 13 A-D).

Equal Detection of Antibody Mediated and Cellular Mediated Acute Rejection Using 17 Genes Via Partial Least Square Discriminant Analysis with Equal Prior Probabilities

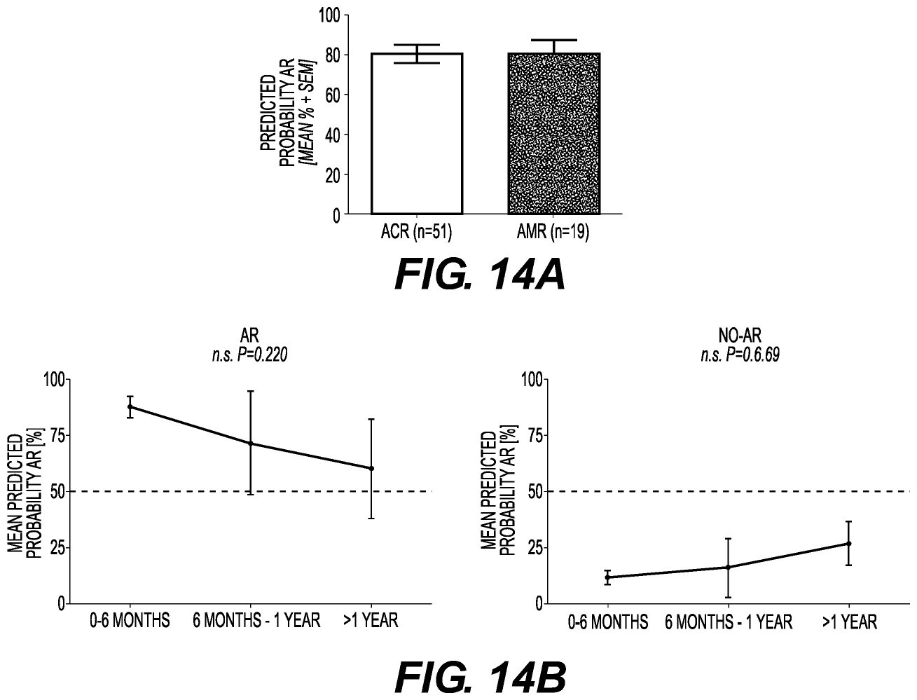

Most of the AR samples analyzed on the Fluidigm platform showed a mixed setting of some cellular and humoral rejection or associated chronic changes. No difference in AR prediction scores between 19 patients with clear antibody mediated rejection only (AMR, C4D+ biopsy staining, DSA+) and 51 patients with clear cellular mediated rejection (ACR, C4d- and DSA−, and Banff t- and i-scores >1) was observed when assessed by the fixed 17 gene-model (plsDA; p=0.9906; mean ACR=80.84%±4.4; mean AMR=80.75%±6.6; FIG. 14A).

Classification of AR is Independent of Time Post Transplantation Using 17 Genes Via Partial Least Square Discriminant Analysis with Equal Prior Probabilities

To evaluate whether time of rejection post transplantation affected the prediction accuracy of the 17 genes, predicted AR probabilities in AR and No-AR samples collected between 0-6 months, 6-12 months, and >1 year post transplantation were evaluated and not found to be impacted by post-transplant time (FIG. 14B).

17 Genes Predict Biopsy Confirmed AR Prior to Clinical Graft Dysfunction in 191 Samples Via Partial Least Square Discriminant Analysis with Equal Prior Probabilities

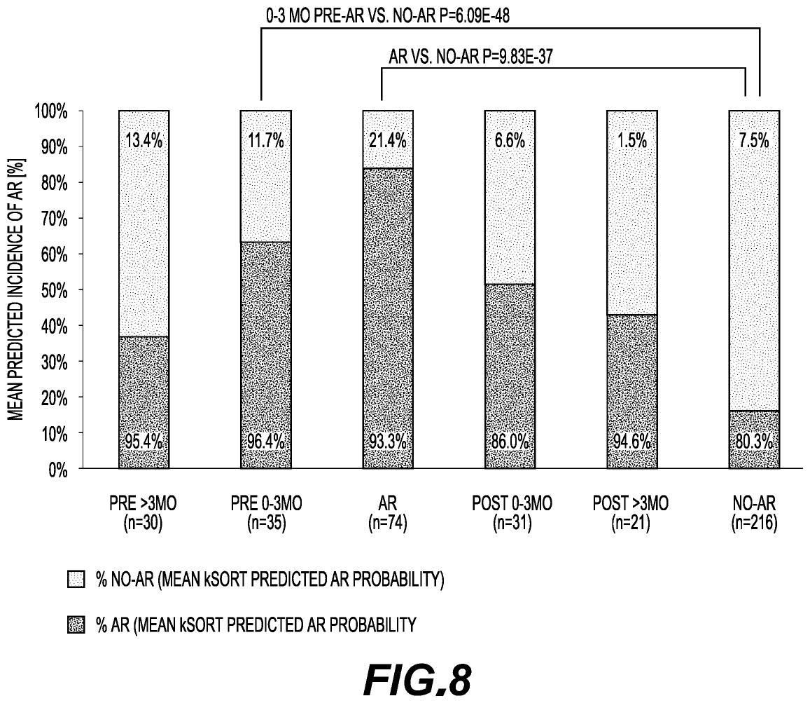

To evaluate the predictive nature of the 17 gene kidney AR prediction assay model, 191 blood samples (Cohort 3; prospective validation) drawn either before (0.2-6.8 months, n=65) or after (0.2-7 months; n=52) a biopsy matched AR episode (n=74) were analyzed. Out of the patients with blood samples 0-3 months prior to the AR biopsy (n=35), at time of stable graft function, 62.9% (22/35) had very high AR prediction scores (96.4%±0.8) (FIG. 8), significantly greater than scores from patients with stable graft function and no AR on follow-up (19.4%±0.3; p<0.0001). Out of the patients with blood samples drawn 0-3 months after AR treatment (n=31), 51.6% (16/31) still had elevated predicted AR scores (86%±0.17); 15/31 samples showed AR scores below the threshold for AR (6.59%±0.13%) at 0-3 months after AR treatment. As serum creatinine levels in patients with elevated AR prediction scores were 2.04±0.4 mg/dL compared to creatinine levels of 1.8±0.4 mg/dL in patients with decreased AR prediction scores, the latter likely represent patients who responded to AR treatment (FIG. 8).

### Example 20: Classification of AR and No-AR Via kSAS

### Selection of ABI Viia7 QPCR Platform for Standard QPCR

High throughput QPCR platforms such as the Fluidigm platform are highly suitable for the discovery and initial development of a diagnostic biomarker panel, but large sample sizes and gene numbers are required in order to provide cost-effective performance. Thus, the 17-gene model was analyzed using 100 samples collected from 44 AR and 56 No-AR patients by standard qPCR (ABI viia7, Life Technologies, Foster City, Calif.) in order to develop a clinically applicable assay having a customizable format and cost-effective performance for variable and smaller sample numbers. In order to optimize these gene sets for clinical analyses (scalability, cost, machine availability, protocol simplicity), the ABI qPCR platform was employed for downstream discovery and validation.

Classification of Adult and Pediatric AR and No-AR Using 10 Genes Via kSAS

For discovery, the kSAS analysis was restricted to two adult centers (UCSF and Pittsburgh) and one pediatric center (SNS). This analysis yielded a 7-gene model (CFLAR, DUSP1, IFNGR1, ITGAX, MAPK9, NKTR, and RYBP) that could classify AR status at a rate of 89% across both adult and pediatric centers. Alternative model sizes of 3-10 genes had overall lower performance than the final set of 7 genes. The combined classification rate for adults yielded 81% accuracy, based on 16 AR and 16 no-AR samples (sensitivity=88%, specificity=75%), and 90% accuracy in the pediatric set based on 22 AR and 155 no-AR samples (sensitivity=91%, sensitivity=90%).

Classification of AR and No-AR Using 17 Genes Via kSAS

In addition to the 10 pediatric genes previously discovered, 7 adult classifier genes identified from the Fluidigm analysis (CEACAM4, EPOR, GZMK, RARA, RHEB, RXRA, and SLC25A37) were added to the ABI gene panel. The sequences of these 17 genes are provided in Appendix A as genomic DNA sequences. Nearly all of these genes were also identified as high-value prognostic markers when re-analyzing the 143 patient Fluidigm qPCR dataset with kSAS. This large ABI gene-set analysis was initially restricted to adult patient blood samples with confirmed AR or no-AR status. In order to identify improved performing gene subsets from these 17 pediatric and adult genes, kSAS qPCR data collected from two centers (UCSF and Pittsburgh) was initially reapplied. Combining overall classification rates from these centers for all possible 17-gene combinations (3-17 genes per model) yielded a set of thirteen models, each containing 12 distinct gene combinations that performed at a rate of 90% (88% sensitivity, 94% specificity) Table 2.

As an independent verification of these gene models, this model set was tested against a new set of adult and pediatric patients (Barcelona and Mexico, respectively), as well as a second set of independent patients from one training center (UCSF). Analysis of UCSF patients using the AR and no-AR expression reference from the discovery analysis (prior UCSF samples) yielded a validation rate ranging from 76-86%. When the results from all new samples were aggregated, a top model classification rate of 88% (86% sensitivity, 90% specificity) resulted, with similar classification rates between adult and pediatric samples. This top 12-gene model (CFLAR, PSEN1, CEACAM4, NAMPT, RHEB, GZMK, NKTR, DUSP1, RARA, ITGAX, SLC25A37, and EPOR) contains 5 genes from the pediatric classification set (CFLAR, PSEN1, NAMPT, NKTR, and DUSP1), and classifies AR status irrespective of age, demographics, induction, maintenance immunosuppression, co-morbidities, or confounding graft pathology. When evaluated in the context of experimentally predicted interactions, more than half of these genes directly or indirectly associated.

Calculation of AR Risk Scores for 100 Adult and Pediatric Samples Using 17 Genes Via kSAS

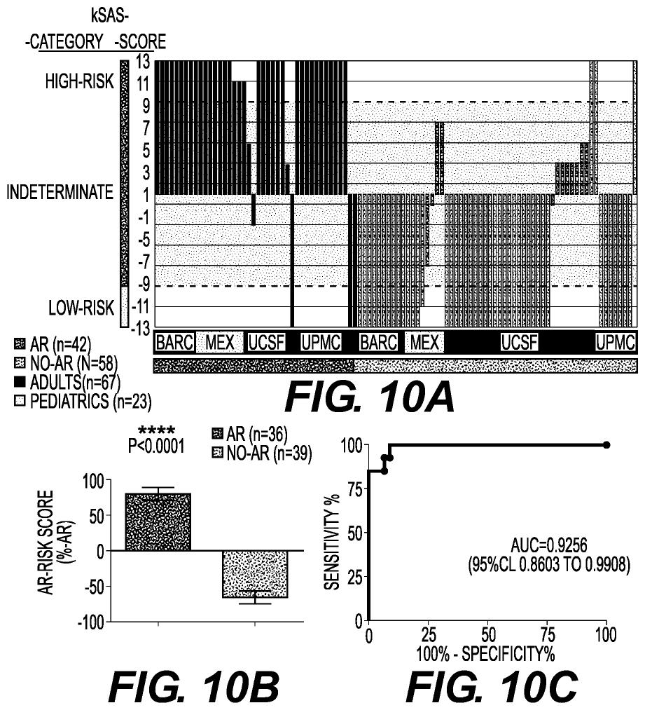

As the multiple model approach provides distinct scores for each gene set, the combined ability of these models to provide a confidence score that is not biased a single gene model for each patient was evaluated. This aggregated AR risk analysis produces a numerical score for each patient (−13 to 13), indicating the risk of AR (13=high risk, −13=very low risk). Among patients with a “high risk of AR”, 91% (31 out of 34) were correctly classified as AR, whereas for patients with a “very low risk of AR”, 92% (35 out of 38) were correctly classified as no-AR. The remainder of the patients (n=15) was predicted with indeterminate risk (FIG. 10A).

Mean calculated AR Risk scores were significantly higher in AR compared to No-AR (p<0.0001) (FIG. 10B) using kSAS.

The calculated AUC was 0.93 (95% CI 0.86-0.99) for the definite kSAS calls (High-Risk AR, Low-Risk AR, n=85) (FIG. 10C).

A strength of the presented assay is its high PPV (92.3%) of detecting AR in a peripheral blood sample. The only diagnostic test that is currently available in transplantation detects the absence of moderate/severe acute cellular cardiac rejection (ISHLT 3A), but performs poorly for detection of the presence of AR (PPV=6.8%) (Deng et al., 2006, Am J. Transplant 6:150-160). Similarly, a blood gene expression test for assessing obstructive coronary artery disease (Corus®Cad, CARDIODX®, Palo Alto, Calif.) yielded a PPV of 46% in a multicenter validation study (Rosenberg et al, 2010, Ann Intern Med 153:425-434). In addition to the high sensitivity of the assay to detect AR at the point of rejection (as diagnosed by the current gold standard), the assay also detected sub-clinical rejection in 12 cases and predicted clinical AR in >60% of samples collected up to 3 months prior to graft dysfunction and histological AR; an important ability of a rejection test, as subclinical and clinical AR are precursors of chronic rejection and graft loss (Nasesens et al., 2012, Am J Transpalnt 12: 2730-2743). Although current immune-monitoring tools for assessing the adaptive alloimmune response, either evaluating circulating donor-specific antibodies or memory-effector T-cells, have shown their usefulness for predicting the potential risk of AR (Loupy et al, 2013, N Engl J Med 369: 1215-1216; and Bestard et al, 2013 Kidney Int 84: 1226-1236), their detection does not necessarily translate to ongoing immune-mediated allograft damage and furthermore, these effector mechanisms may not always be detected prior to or at the time of biopsy proven AR. Furthermore, most centers currently do not perform protocol biopsies as a means to detect subclinical AR, and thus these remain largely undetected. Routine post-transplant monitoring with the assay provided herein can predict AR, limit tissue damage with timely intervention and can reduce the financial burden on the health system by minimizing the numbers of patients that will return to cost intensive dialysis.

### Example 21: Biology of 17 Genes for AR and No-AR Classification

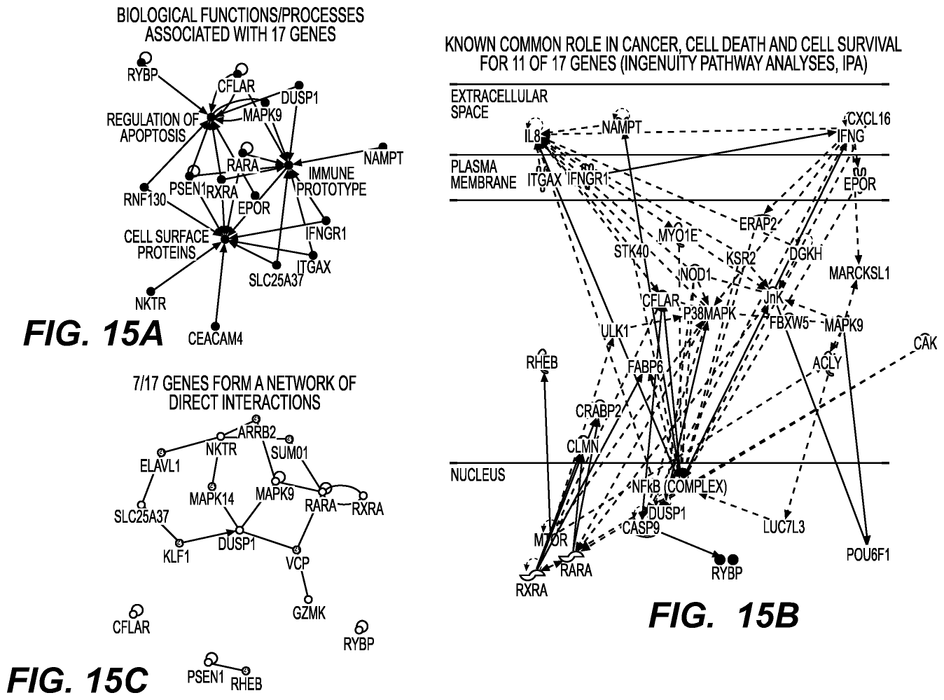

When evaluated in the context of experimentally predicted interactions, more than half of genes were directly or indirectly associated with each other by common molecular pathways (FIG. 15a-15c), particularly, regulation of Apoptosis, Immune Phenotype and Cell Surface. In addition to the 10 genes previously evaluated as peripheral biomarkers for pediatric AR, and known to be mostly expressed in peripheral blood cells of the monocyte lineage, expression of 6 of the additional peripheral 7 AR genes were also expressed by activated monocytes (RXRA, RARA, CEACAM4), endothelial cells (EPOR, SLC25A37) and T-cells (GZMK) in the peripheral circulation. Eleven of the 17 genes played a common role in a Cell Death, and Cell Survival Network (Fisher's exact test, p<0.05; IPA; FIG. 15c).

### Example 21: Identification of Common Rejection Module (CRM) Using Leave-One-Organ-Out Analysis

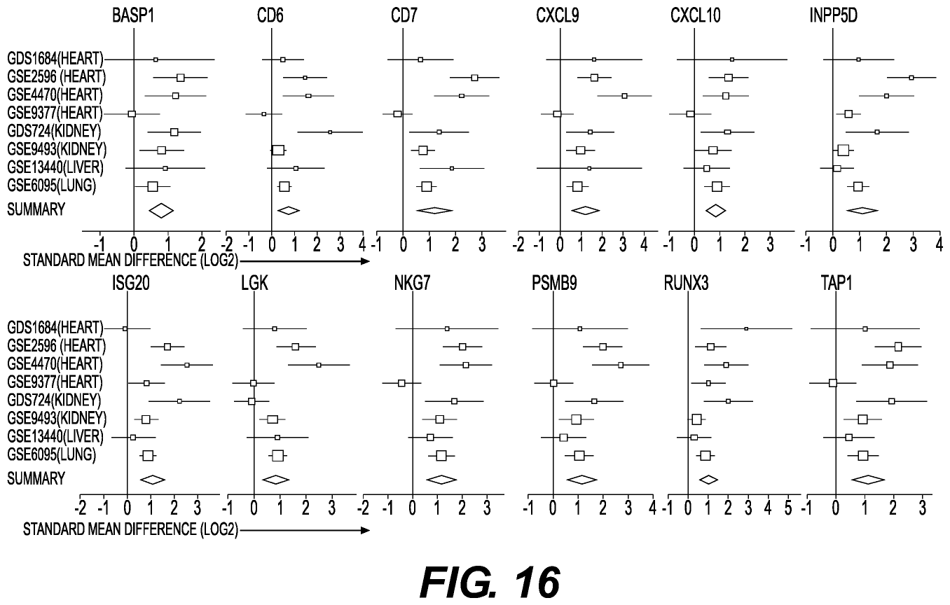

A common rejection module was identified by analyzing the whole genome expression data from 236 independent biopsy samples from kidney, lung, heart, and liver transplant patients. Each dataset was gcRMA normalized (see, Irizarray, E. et al. Nucleic Acids Res. 2003, 31, e15). Transplant databases were analyzed by meta-analysis methods of combining size effect and combining p-values identifying 102 genes (listed in Table 3) at a FDR of 20%. Iterations of removing one organ at a time resulting in iterative combinations of the different organs were each analyzed by meta-analysis revealing 12 genes comprising BASP1, CD6, CD7, CXCL10, CXCL9, INPP5D, ISG20, LCK, NKG7, PSMB9, RUNX3, and TAP1 overexpressed in all organs (FIG. 16).

1-87. (canceled)

88. A method of detecting transplant rejection in a solid organ transplant recipient, the method comprising
a) performing polymerase chain reaction (PCR) to amplify cDNA that is generated from mRNA isolated from a biological sample obtained from the recipient, wherein the PCR amplification targets cDNA encoding genes comprising CFLAR, NKTR, PSEN1, ITGAX, DUSP1, RYBP, MAPK9, and NAMPT
b) assessing levels of the amplified PCR products to determine expression levels in the recipient of genes comprising CFLAR, NKTR, PSEN1, ITGAX, DUSP1, RYBP, MAPK9, and NAMPT,
wherein increased expression levels of each of the genes over normal expression levels of each of the genes is indicative of transplant rejection in recipient.

89. The method of claim 88, wherein assessing level of the amplified PCR products is quantitative.

90. The method of claim 89, wherein the sample obtained from the recipient is selected from the group consisting of whole blood, peripheral blood leukocytes and peripheral blood mononuclear cells.

91. The method of claim 90, wherein the wherein the PCR amplification targets cDNA encoding genes further comprising CEACAM4, EPOR, GZMK, RARA, RHEB, RXRA and SLC25A37.

92. The method of claim 91, wherein the recipient is a kidney transplant recipient.

93. The method of claim 92, wherein the PCR amplification targets cDNA encoding genes consisting of CFLAR, NKTR, PSEN1, ITGAX, DUSP1, RYBP, MAPK9, NAMPT, CEACAM4, EPOR, GZMK, RARA, RHEB, RXRA and SLC25A37.

94. The method of claim 92, wherein assessing levels of the amplified PCR products to determine expression levels in the recipient of genes consists of the genes CFLAR, NKTR, PSEN1, ITGAX, DUSP1, RYBP, MAPK9, NAMPT, CEACAM4, EPOR, GZMK, RARA, RHEB, RXRA and SLC25A37.

95. The method of claim 94, wherein assessing levels of the amplified PCR products occurs before the onset of any detectable symptoms of transplant rejection.

96. The method of claim 94, wherein normal expression levels of each of the genes is assessed in the recipient prior to receiving the transplant.

97. The method of claim 94, wherein normal expression levels of each of the genes is assessed in one or more non-recipients.

98. The method of claim 94, wherein the transplant rejection is acute rejection.

99. The method of claim 90, wherein the PCR amplification targets cDNA encoding genes further comprising IFNGR1 and RNF130.

100. The method of claim 99, wherein the recipient is a heart transplant recipient.

101. The method of claim 100, wherein the PCR amplification targets cDNA encoding genes consisting of CFLAR, NKTR, PSEN1, ITGAX, DUSP1, RYBP, MAPK9, NAMPT, IFNGR1 and RNF130.

102. The method of claim 100, wherein assessing levels of the amplified PCR products to determine expression levels in the recipient of genes consists of the genes CFLAR, NKTR, PSEN1, ITGAX, DUSP1, RYBP, MAPK9, NAMPT, IFNGR1 and RNF130.

103. The method of claim 102, wherein assessing levels of the amplified PCR products occurs before the onset of any detectable symptoms of transplant rejection.

104. The method of claim 102, wherein normal expression levels of each of the genes is assessed in the recipient prior to receiving the transplant.

105. The method of claim 102, wherein normal expression levels of each of the genes is assessed in one or more non-recipients.

106. The method of claim 102, wherein the transplant rejection is acute rejection.
